(19) United States
(12) Patent Application Publication
(10) Pub. No.: US 2011/0275721 A1
(43) Pub. Date: Nov. 10, 2011

(54) METHOD FOR THE PURIFICATION OF SUBSTITUTED CYCLOPENT-2-EN-1-ONE CONGENERS AND SUBSTITUTED 1,3-CYCLOPENTADIONE CONGENERS FROM A COMPLEX MIXTURE USING COUNTERCURRENT SEPARATION

(75) Inventors: Brian J. Carroll, Gig Harbor, WA (US); Clinton J. Dahlberg, Port Orchard, WA (US); James S. Traube, Boulder, CO (US)

(73) Assignee: Metaproteomics, LLC, San Clemente, CA (US)

(21) Appl. No.: 13/132,492

(22) PCT Filed: Dec. 10, 2009
(86) PCT No.: PCT/US09/67460

§ 371 Date: Jul. 25, 2011

Related U.S. Application Data
(60) Provisional application No. 61/121,419, filed on Dec. 10, 2008.

(51) Int. Cl. A61K 31/122; C07C 45/80

(52) U.S. Cl. 514/690; 568/366

(57) ABSTRACT

Methods of purifying individual congeners of substituted cyclohexa-2,4-dienones, substituted cyclohexane-1,3,5-triones, substituted cyclopent-2-en-1-ones, and substituted 1,3-cyclopentadiones and compositions using the same are disclosed. The purification method includes the steps of: (a) selecting a congener to be isolated; (b) dissolving the mixture of congeners in a bi-phasic solvent system specific to the selected congener to be isolated, wherein said bi-phasic system has a partition coefficient from about 0.5 to 5.0; (c) subjecting the mixture dissolved in the bi-phasic solvent system to a counter current chromatography; and (d) isolating the selected congener in substantially pure form.

### CROSS-REFERENCE TO RELATED APPLICATIONS

This patent application is a non-provisional application of, and claims priority to and the benefit of, U.S. Provisional Patent Application Ser. No. 60/121,419, filed on Dec. 10, 2008, the entire contents of which are incorporated herein.

### BACKGROUND OF THE INVENTION

1. Field of the Invention

The present invention relates to the field of countercurrent chromatographic purification; in particular, the present invention relates to methods of purifying individual congeners of substituted cyclohexa-2,4-dienones, substituted cyclohexane-1,3,5-triones, substituted cyclopent-2-en-1-ones, and substituted 1,3-cyclopentadiones present in a synthetic mixture or which may either be isolated or derived from hops. Further disclosed are compositions utilizing these substantially pure congeners. These substantially pure congeners are obtained through the use of an appropriate immiscible solvent system and countercurrent separation instrumentation.

2. Description of the Related Art

Recently it has been reported that a mixture of congeners of iso-alpha acids (substituted cyclopent-2-en-1-ones) produce beneficial effects in murine models of obesity and dyslipidemia. In addition to these reports a mixture of congeners belonging to a separate family of iso-alpha acids (substituted cyclopent-2-en-1-ones), the reduced iso-alpha acids (substituted 1,3-cyclopentadiones), possess anti-inflammatory activity in vitro and have clinically demonstrated effectiveness in reducing arthritic knee pain. In order to determine the differences in biological activity between the various iso-alpha acid congeners (substituted cyclopent-2-en-1-ones) as well as the congeners belonging to the family of reduced iso-alpha acids (substituted cyclopent-2-en-1-ones), an efficient purification method for that makes it possible to purify these congeners is required.

The earliest reported methods for purifying congeners of the iso-alpha relied upon a Craig-Post countercurrent distribution (CCD) apparatus as reported by Rigby and Bethune (Rigby, F. L. and Bethune, J. L. (1952) Proc. Am. Soc. Brew. Chem. 98-105.). In this report, the iso-alpha acid congener purified was referred to as “isohumulone” and was identified based upon its identical physical properties compared to a reference sample synthesized from the precursor referred to as “humulone”. Additionally, a second congener, referred to as “isocohumulone” was isolated. Using a CCD approach similar to the one reported by Rigby & Bethune, Verzele et al., (Verzele M., Anteunis, M. and Alderweireldt F., J. Inst. Brewing, (1965), 71, 232) reported the purification of two novel congeners of iso-alpha acids referred to as “trans-isohumulone” and “cis-isohumulone”. Additional examples and references to published reports of the purification of various congeners of iso-alpha or reduced iso-alpha acids using the CCD approach can be found in the seminal book by Verzele and DeKeukeleire. (M. Verzele, D. DeKeukeleire, Chemistry and Analysis of Hop and Beer Bitter Acids. Elsevier: New York, 1991; Vol. 27.).

Innovations and improvements in the technology of countercurrent separation (CS) have been reviewed by Pauli et al, (J. Nat. Prod., 2008, 71(8), pp 1489-1508). As discussed by Pauli et al and shown in FIG. 18, the proliferation of unique and innovative CS designs is a notable and recent developments in the area of CS. Because of the advantages that are inherent and fundamental to the design and operation of these contemporary instrumentations, the aforementioned CCD apparatus originally described by Rigby and Bethune, and Verzele et al, has been rendered obsolete. According to Pauli et al., contemporary CS instruments operate according to one of two canonical design principles i.e., they are either hydrostatic or hydrodynamic designs. These two designs are distinct from each other and—compared to the now obsolete CCD instruments used by Verzele et al and their contemporaries—they are fundamentally unique in their design and inherently superior in their operation, efficiency and performance.

Hops have been used for decades to flavor beer and are considered, along with water, yeast and malt, to be an essential ingredient of beer. Various studies have shown that reduced isoalpha acids derived from hops are useful as anti-inflammatory agents and dietary supplements. Recent studies have further shown that certain isomers of the reduced isoalpha acids can be more effective therapeutically than racemic mixtures.

### SUMMARY OF THE INVENTION

There exists a need for an efficient, accurate and relatively inexpensive method of purification of isomers of substituted cyclohexa-2,4-dienones, substituted cyclohexane-1,3,5-triones, substituted cyclopent-2-en-1-ones, and substituted 1,3-cyclopentadiones. The present invention using countercurrent separation further provides compositions comprising these congeners in substantially pure form.

A first aspect of the invention discloses methods for isolating a congener in substantially pure form from a mixture of congeners comprising the steps of:
a. selecting a congener to be isolated;
b. dissolving the mixture of congeners in a bi-phasic solvent system specific to the selected congener to be isolated, wherein said bi-phasic solvent system has a partition coefficient from about 0.5 to 5.0;
c. subjecting the mixture of congeners dissolved in the bi-phasic solvent system to a counter current chromatography; and
d. isolating the selected congener;
   where in this aspect the congener is selected from the group consisting of substituted cyclohexa-2,4-dienones, substituted cyclohexane-1,3,5-triones, substituted cyclopent-2-en-1-ones, and substituted 1,3-cyclopentadiones.

A second aspect of the invention discloses compositions comprising a substantially pure congener or a pharmaceutically acceptable salt thereof, wherein the congener is obtained from a mixture of congeners comprising the steps of:
a. selecting a congener to be isolated;
b. dissolving the mixture of congeners in a bi-phasic solvent system specific to the selected congener to be isolated, wherein said bi-phasic solvent system has a partition coefficient from about 0.5 to 5.0;
c. subjecting the mixture of congeners dissolved in the bi-phasic solvent system to a counter current chromatography; and
d. isolating the selected congener;
   where in this aspect the congener is selected from the group consisting of substituted cyclohexa-2,4-dienones, substituted cyclohexane-1,3,5-triones, substituted cyclopent-2-en-1-ones, and substituted 1,3-cyclopentadiones.

A third aspect discloses a method for isolating a congener in substantially pure form from a mixture of congeners comprising the steps of:
a. preparing a suitable mixture of individual congeners derived from an extract of hops;
b. dissolving said mixture with a suitable solvent that can be introduced into a counter current separation instrument for the purpose of purification;
c. collecting a homogenous or partially homogenous solution of individual congeners;
d. extracting the counter current separation purified congeners into a suitable solvent obtained from step (c); or
e. removing the solvent of the solution obtained in step (c) of a homogenous or partially homogenous solution of an individual congener to render the pure or partially pure congener; and
f. removing the solvent of the solution obtained in step (d) of a homogenous or partially homogenous solution of an individual congener to render the pure or partially pure congener;
   wherein said congener is selected from the group consisting of substituted cyclohexa-2,4-dienones, substituted cyclohexane-1,3,5-triones, substituted cyclopent-2-en-1-ones, and substituted 1,3-cyclopentadiones.

A fourth aspect discloses compositions comprising a substantially pure congener or a pharmaceutically acceptable salt thereof, wherein the congener is obtained from a mixture of congeners comprising the steps of:
a. preparing a suitable mixture of individual congeners derived from an extract of hops;
b. dissolving said mixture with a suitable solvent that can be introduced into a counter current separation instrument for the purpose of purification;
c. collecting a homogenous or partially homogenous solution of individual congeners;
d. extracting the counter current separation purified congeners into a suitable solvent obtained from step (c); or
e. removing the solvent of the solution obtained in step (c) of a homogenous or partially homogenous solution of an individual congener to render the pure or partially pure congener; and
f. removing the solvent of the solution obtained in step (d) of a homogenous or partially homogenous solution of an individual congener to render the pure or partially pure congener;
   wherein said congener is selected from the group consisting of substituted cyclohexa-2,4-dienones, substituted cyclohexane-1,3,5-triones, substituted cyclopent-2-en-1-ones, and substituted 1,3-cyclopentadiones.

### BRIEF DESCRIPTION OF THE DRAWINGS

FIG. 1 shows the cis and trans diastereomers of tetrahydro isoalpha acids (THIAA).

FIG. 2 depicts a reaction scheme for conversion of alpha acids to isoalpha acids followed by conversion to tetrahydro isoalpha acids.

FIG. 3 depicts a chromatogram showing the composition of THIAA prior to high-speed counter current chromatography (HSCCC) separation: each peak, in order from left to right, represents the followings: TH1: cis tetra co-isoalpha acid; TH2: trans tetra co-isoalpha acid; TH3: trans tetra ad-isoalpha acid; TH4: cis tetra-ad-isoalpha acid; TH5: cis tetra n-isoalpha acid, TH6: trans tetra n-isoalpha acid.

FIG. 4 depicts a chromatogram showing the elution and fractionation of THIAA components (via UV monitoring at 254 nm). This separation was performed in descending mode using a coil volume of 325 mL; 350 mg total amount injected.

FIG. 5 shows a representative chromatogram of a THIAA composition. The top panel identifies the chromatographic peaks comprising the THIAA components of the mixture. The bottom table depicts the chemical structure of individual members forming THIAA. Each peak in the chromatogram corresponds to a different THIAA compound with a different R group shown in the table. The location of the R group is shown in the THIAA structure depicted in the upper panel.

FIG. 6 depicts a representative chromatogram of a THIAA composition. The top panel identifies the chromatographic peaks comprising the THIAA components of the mixture whereas the subsequent panels show the chromatographic profile of each individual and isolation fraction and the corresponding structure.

FIG. 7 shows NMR data obtained for each of the TH (THIAA diastereomers) components purified. Panels A-E depict TH1, TH2, TH4, TH5, and TH7 respectively.

FIG. 8 depicts the chemical structures of the major components found in cis tetrahydro isoalpha acid raw material.

FIG. 9 is an HPCCC chromatogram with the following parameters: 100 mg cis tetrahydro isoalpha acids; configuration: J-type planetary; mode: descending (head-to-tail); stationary phase: hexanes; mobile phase: 250 mM NH₄PO₄ (aq), pH 6.3; injection volume: 10 mL.

FIG. 10 is an HPCCC chromatogram with the following parameters: 1021 mg cis tetrahydro isoalpha acids; configuration: J-type planetary; mode: descending (head-to-tail); stationary phase: hexanes; mobile phase: 250 mM NH₄PO₄ (aq), pH 6.3; injection volume: 100 mL (10 mg/mL); coil volume: 810 mL; RPM: 700; flow rate: 4 mL/min; stationary phase retention: 65%; run time: 650 min.

FIG. 11 is an HPCCC chromatogram of FIG. 10 with bars showing the amount of each of the three major fractions collected and the percent homogeneity of each fraction.

FIG. 12 is an HPLC chromatogram of the tetrahydro cis n isoalpha acid raw material showing structures and inlaid characteristic UV spectra.

FIG. 13 is an HPCCC chromatogram with the following parameters: 100 mg (10 mg/ml), 6 mL/min.

FIG. 14 is an overlay of HPLC chromatograms of the fractions purified by HPCCC in Example 2.

FIG. 15 depicts the chromatograms in FIG. 14 in stack format.

FIG. 16 shows the result of an HSCCC purification process using specific parameters depicted (i.e., THIAA pH 6.3; 250 mM NH4PO4; analytical coils; 2 mL/min at 700 rpm; 61% stationary retention).

FIG. 17 is a picture of the chemical structures of the predominant iso-alpha or reduced iso-alpha acids congeners that are found in extracts and modified extracts derived from hops (Humulus Lupulus L.).

FIG. 18 is a diagram classifying contemporary CS instrument designs.

FIG. 19 is a flowchart showing the procedure for purifying two individual congeners, IA1, and IA5 to 99% homogeneity, respectively, from a mixture of iso-alpha acids congeners present in Isohop®.

FIG. 20 is a flowchart showing the procedure for purifying two individual congeners, TH1, and TH5 to 99% homogeneity, respectively, from a mixture of cis tetrahydro iso-alpha acids.

FIG. 21 is an HPLC chromatogram of a representative mixture of iso-alpha acids congeners present in Isohop®

FIG. 22 is an HPLC chromatogram of a representative mixture of cis tetrahydro iso-alpha acids

FIG. 23 is a reconstructed trace for a countercurrent separation (CS) according to the description provided in Example 4 for the purification of cis tetrahydro iso-alpha acid congeners.

FIG. 24 is a reconstructed trace for a countercurrent separation (CS) according to the description provided in Example 5 for the purification of cis tetrahydro iso-alpha acid congeners.

FIG. 25 is a reconstructed trace for a countercurrent separation (CS) according to the description provided in Example 6 for the purification of cis iso-alpha acid congeners.

FIG. 26 depicts the complex equilibria between a specific congener of iso alpha acid (or reduced iso-alpha acid) and any salt or buffers present in the system.

FIG. 27 shows the concentration of buffer greatly impacts the partitioning of IAA congeners IA1, IA5 and IA4.

FIG. 28 illustrates the effects of the stoichiometry between the buffer and two IA congeners.

FIG. 29 shows how the amount of buffer affects the pH in the two solvent systems SS1 (HexWat) and SS2 and (Hemwat) for two IAA congeners.

### DETAILED DESCRIPTION OF THE INVENTION

The present invention provides a process for the preparative chromatographic isolation or purification of structural analogs of substituted cyclohexa-2,4-dienones, substituted cyclohexane-1,3,5-triones, substituted cyclopent-2-en-1-ones, and substituted 1,3-cyclopentadiones, and their respective cis/trans diastereomers. Further disclosed are compositions utilizing these substantially pure congeners.

The patents, published applications, and scientific literature referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of DNA technology include Sambrook et al., Molecular Cloning: A Laboratory Manual, 2nd Ed., Cold Spring Harbor Laboratory Press, New York (1989); and Kaufman et al., Eds., Handbook of Molecular and Cellular Methods in Biology in Medicine, CRC Press, Boca Raton (1995). Standard reference works setting forth the general principles of pharmacology include Goodman and Gilman's The Pharmacological Basis of Therapeutics, 11th Ed., McGraw Hill Companies Inc., New York (2006).

In the specification and the appended claims, the singular forms include plural referents unless the context clearly dictates otherwise. As used in this specification, the singular forms “a,” “an” and “the” specifically also encompass the plural forms of the terms to which they refer, unless the content clearly dictates otherwise. Additionally, as used herein, unless specifically indicated otherwise, the word “or” is used in the “inclusive” sense of “and/or” and not the “exclusive” sense of “either/or.” The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the recitation of a numerical range for a variable is intended to convey that the invention may be practiced with the variable equal to any of the values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of recombinant DNA technology include Sambrook et al., Molecular Cloning: A Laboratory Manual, 2nd Ed., Cold Spring Harbor Laboratory Press, New York (1989); Kaufman et al., Eds., Handbook of Molecular and Cellular Methods in Biology in Medicine, CRC Press, Boca Raton (1995); McPherson, Ed., Directed Mutagenesis: A Practical Approach, IRL Press, Oxford (1991). Standard reference works setting forth the general principles of pharmacology include Goodman and Gilman's The Pharmacological Basis of Therapeutics, 11th Ed., McGraw Hill Companies Inc., New York (2006).

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

The patents, published applications, and scientific literature referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

Reference is made hereinafter in detail to specific embodiments of the invention. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the appended claims. In the following description, numerous specific details are set forth in order to provide a thorough understanding of the present invention. The present invention may be practiced without some or all of these specific details. In other instances, well known process operations have not been described in detail, in order not to unnecessarily obscure the present invention.

Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The methods and compositions of the present invention are intended for use with any mammal that may experience the benefits of the methods of the invention. Foremost among such mammals are humans, although the invention is not intended to be so limited, and is applicable to veterinary uses. Thus, in accordance with the invention, “subjects in need” include humans as well as non-human mammals, particularly domesticated animals including, without limitation, cats, dogs, and horses. “Subjects in need” additionally encompasses reptiles, birds, fish, and amphibians.

Reference is made hereinafter in detail to specific aspects and embodiments of the invention. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the appended claims. In the following description, numerous specific details are set forth in order to provide a thorough understanding of the present invention. The present invention may be practiced without some or all of these specific details. In other instances, well known process operations have not been described in detail, in order not to unnecessarily obscure the present invention.

A first aspect of the invention discloses methods for isolating a congener in substantially pure form from a mixture of congeners comprising the steps of:
a. selecting a congener to be isolated;
b. dissolving the mixture of congeners in a bi-phasic solvent system specific to the selected congener to be isolated, wherein said bi-phasic solvent system has a partition coefficient from about 0.5 to 5.0;
c. subjecting the mixture of congeners dissolved in the bi-phasic solvent system to a counter current chromatography; and
d. isolating the selected congener;
   where in this aspect the congener is selected from the group consisting of substituted cyclohexa-2,4-dienones, substituted cyclohexane-1,3,5-triones, substituted cyclopent-2-en-1-ones, and substituted 1,3-cyclopentadiones.

As used herein, “substantially pure” shall mean isolates wherein the named, referent congener is present at greater than 65% purity. Preferably, the purity shall be greater than 80% for use in nutraceutical, medical foods, or dietary supplement uses. Most preferred shall be purities greater than 95%, suitable for pharmaceutical drug use applications.

As used herein, the term “dietary supplement” refers to compositions consumed to affect structural or functional changes in physiology.

In some embodiments of this aspect, the substituted cyclohexa-2,4-dienone is selected from the group consisting of (6R)-3,5,6-trihydroxy-2-(3-methylbutanoyl)-4,6-bis(3-methylbut-2-en-1-yl)cyclohexa-2,4-dien-1-one, (6R)-3,5,6-trihydroxy-4,6-bis(3-methylbut-2-en-1-yl)-2-(2-methylpropanoyl)cyclohexa-2,4-dien-1-one, and (6R)-3,5,6-trihydroxy-2-(2-methylbutanoyl)-4,6-bis(3-methylbut-2-en-1-yl)cyclohexa-2,4-dien-1-one.

As used herein, “substituted cyclohexa-2,4-dienones” refers to those compounds generally described as alpha acid commonly associated with hops and beer production. Examples of substituted cyclohexa-2,4-dienones include, without limitation those compounds identified in Table 1 and their derivatives. The substituted cyclohexa-2,4-dienones may be prepared de novo through chemical synthesis or isolated, derived of modified from materials from hops (Humulus lupulus).

TABLE 1
Substituted cyclohexa-2,4-dienones

| Chemical Name | Structure |
|---|---|
| (6R)-3,5,6-trihydroxy-2-(3- methylbutanoyl)-4,6-bis(3- methylbut-2-en-1- yl)cyclohexa-2,4-dien-1-one | 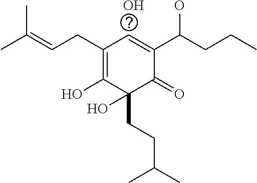 |
| (6R)-3,5,6-trihydroxy-4,6- bis(3-methylbut-2-en-1-yl)- 2-(2- methylpropanoyl)cyclohexa- 2,4-dien-1-one | 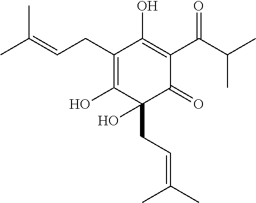 |
| (6R)-3,5,6-trihydroxy-2-(2- methylbutanoyl)-4,6-bis(3- methylbut-2-en-1- yl)cyclohexa-2,4-dien-1-one | 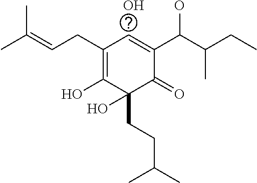 |

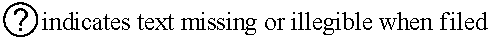

In further embodiments, the substituted cyclohexane-1,3,5-trione is selected from the group consisting of substituted cyclohexane-1,3,5-triones wherein said composition is enriched for one or more compounds selected from the group consisting of 3,5-dihydroxy-2-(3-methylbutanoyl)-4,6,6-tris(3-methylbut-2-en-1-yl)cyclohexa-2,4-dien-1-one, 3,5-dihydroxy-4,6,6-tris(3-methylbut-2-en-1-yl)-2-(2-methylpropanoyl)cyclohexa-2,4-dien-1-one, and 3,5-dihydroxy-2-(2-methylbutanoyl)-4,6,6-tris(3-methylbut-2-en-1-yl)cyclohexa-2,4-dien-1-one.

As used herein, “substituted cyclohexane-1,3,5-triones” refers to those compounds generally described as beta acid commonly associated with hops and beer production. Examples of substituted cyclohexane-1,3,5-triones include, without limitation those compounds identified in Table 2 and their derivatives. The substituted cyclohexane-1,3,5-triones may be prepared de novo through chemical synthesis or isolated, derived of modified from materials from hops (Humulus lupulus).

TABLE 2
Substituted cyclohexane-1,3,5-triones

| Chemical Name | Structure |
|---|---|
| 3,5-dihydroxy-2-(3- methylbutanoyl)-4,6,6-tris(3- methylbut-2-en-1- yl)cyclohexa-2,4-dien-1-one | 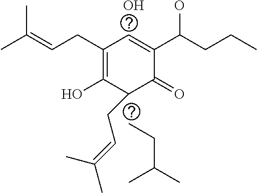 |
| 3,5-dihydroxy-4,6,6-tris(3- methylbut-2-en-1-yl)-2-(2- methylpropanoyl)cyclohexa- 2,4-dien-1-one | 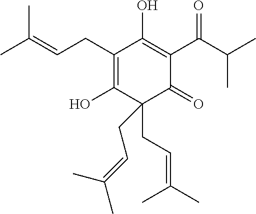 |
| 3,5-dihydroxy-2-(2- methylbutanoyl)-4,6,6-tris(3- methylbut-2-en-1- yl)cyclohexa-2,4-dien-1-one | 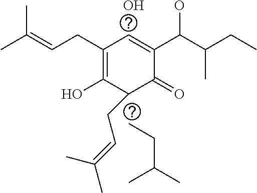 |

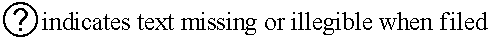

In still other embodiments, the substituted cyclopent-2-en-1-one is selected from the group consisting of (4R,5S)-4-hydroxy-3-methyl-2-(3-methylbutanoyl)-5-(3-methylbut-2-en-1-yl)-4-(4-methylpent-3-enoyl)cyclopent-2-en-1-one, (4S,5S)-4-hydroxy-3-methyl-2-(3-methylbutanoyl)-5-(3-methylbut-2-en-1-yl)-4-(4-methylpent-3-enoyl)cyclopent-2-en-1-one, (4S,5R)-4-hydroxy-3-methyl-2-(3-methylbutanoyl)-5-(3-methylbut-2-en-1-yl)-4-(4-methylpent-3-enoyl)cyclopent-2-en-1-one, (4R,5R)-4-hydroxy-3-methyl-2-(3-methylbutanoyl)-5-(3-methylbut-2-en-1-yl)-4-(4-methylpent-3-enoyl)cyclopent-2-en-1-one, (4R,5S)-4-hydroxy-3-methyl-5-(3-methylbut-2-en-1-yl)-4-(4-methylpent-3-enoyl)-2-(2-methylpropanoyl)cyclopent-2-en-1-one, (4S,5S)-4-hydroxy-3-methyl-5-(3-methylbut-2-en-1-yl)-4-(4-methylpent-3-enoyl)-2-(2-methylpropanoyl)cyclopent-2-en-1-one, (4S,5R)-4-hydroxy-3-methyl-5-(3-methylbut-2-en-1-yl)-4-(4-methylpent-3-enoyl)-2-(2-methylpropanoyl)cyclopent-2-en-1-one, (4R,5R)-4-hydroxy-3-methyl-5-(3-methylbut-2-en-1-yl)-4-(4-methylpent-3-enoyl)-2-(2-methylpropanoyl)cyclopent-2-en-1-one, (4R,5S)-4-hydroxy-3-methyl-2-(2-methylbutanoyl)-5-(3-methylbut-2-en-1-yl)-4-(4-methylpent-3-enoyl)cyclopent-2-en-1-one, (4S,5S)-4-hydroxy-3-methyl-2-(2-methylbutanoyl)-5-(3-methylbut-2-en-1-yl)-4-(4-methylpent-3-enoyl)cyclopent-2-en-1-one, (4S,5R)-4-hydroxy-3-methyl-2-(2-methylbutanoyl)-5-(3-methylbut-2-en-1-yl)-4-(4-methylpent-3-enoyl)cyclopent-2-en-1-one, and (4R,5R)-4-hydroxy-3-methyl-2-(2-methylbutanoyl)-5-(3-methylbut-2-en-1-yl)-4-(4-methylpent-3-enoyl)cyclopent-2-en-1-one.

As used herein, “substituted cyclopent-2-en-1-one” refers to those compounds generally described as isoalpha acids commonly associated with hops and beer production. Examples of substituted cyclohexane-1,3,5-triones include, without limitation those compounds identified in Table 3 and their derivatives. The substituted cyclopent-2-en-1-one may be prepared de novo through chemical synthesis or isolated, derived of modified from materials from hops (Humulus lupulus).

TABLE 3
Substituted cyclopent-2-en-1-ones

| | Syno- | |
|---|---|---|
| Chemical Name | nym | Structure |
| (4R,5S)-4-hydroxy-3- methyl-2-(3- methylbutanoyl)-5-(3- methylbut-2-en-1-yl)-4-(4- methylpent-3- enoyl)cyclopent-2-en-1-one | cis n iso- alpha acid | 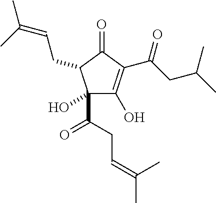 |
| (4S,5S)-4-hydroxy-3- methyl-2-(3- methylbutanoyl)-5-(3- methylbut-2-en-1-yl)-4-(4- methylpent-3- enoyl)cyclopent-2-en-1-one | trans n iso- alpha acid | 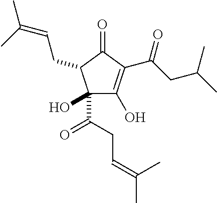 |
| (4S,5R)-4-hydroxy-3- methyl-2-(3- methylbutanoyl)-5-(3- methylbut-2-en-1-yl)-4-(4- methylpent-3- enoyl)cyclopent-2-en-1-one | cis n iso- alpha acid | 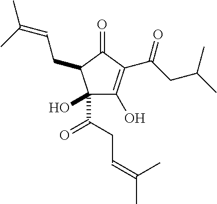 |
| (4R,5R)-4-hydroxy-3- methyl-2-(3- methylbutanoyl)-5-(3- methylbut-2-en-1-yl)-4-(4- methylpent-3- enoyl)cyclopent-2-en-1-one | trans n iso- alpha acid | 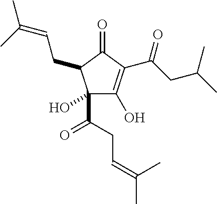 |
| (4R,5S)-4-hydroxy-3- methyl-5-(3-methylbut-2-en- 1-yl)-4-(4-methylpent-3- enoyl)-2-(2- methylpropanoyl)cyclopent- 2-en-1-one | cis co iso- alpha acid | 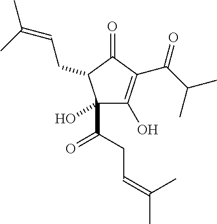 |
| (4S,5S)-4-hydroxy-3- methyl-5-(3-methylbut-2-en- 1-yl)-4-(4-methylpent-3- enoyl)-2-(2- methylpropanoyl)cyclopent- 2-en-1-one | trans co iso- alpha acid | 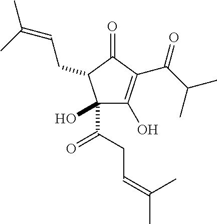 |
| (4S,5R)-4-hydroxy-3- methyl-5-(3-methylbut-2-en- 1-yl)-4-(4-methylpent-3- enoyl)-2-(2- methylpropanoyl)cyclopent- 2-en-1-one | cis co iso- alpha acid | 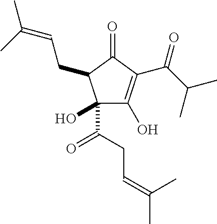 |
| (4R,5R)-4-hydroxy-3- methyl-5-(3-methylbut-2-en- 1-yl)-4-(4-methylpent-3- enoyl)-2-(2- methylpropanoyl)cyclopent- 2-en-1-one | trans co iso- alpha acid | 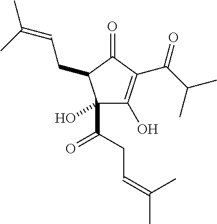 |
| (4R,5S)-4-hydroxy-3- methyl-2-(2- methylbutanoyl)-5-(3- methylbut-2-en-1-yl)-4-(4- methylpent-3- enoyl)cyclopent-2-en-1-one | cis ad iso- alpha acid | 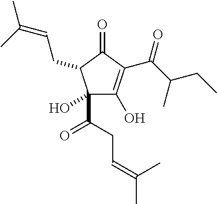 |
| (4S,5S)-4-hydroxy-3- methyl-2-(2- methylbutanoyl)-5-(3- methylbut-2-en-1-yl)-4-(4- methylpent-3- enoyl)cyclopent-2-en-1-one | trans ad iso- alpha acid | 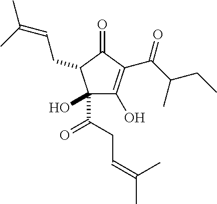 |
| (4S,5R)-4-hydroxy-3- methyl-2-(2- methylbutanoyl)-5-(3- methylbut-2-en-1-yl)-4-(4- methylpent-3- enoyl)cyclopent-2-en-1-one | cis ad iso- alpha acid | 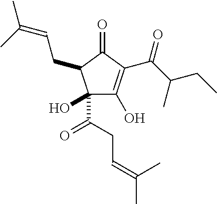 |
| (4R,5R)-4-hydroxy-3- methyl-2-(2- methylbutanoyl)-5-(3- methylbut-2-en-1-yl)-4-(4- methylpent-3- enoyl)cyclopent-2-en-1-one | trans ad iso- alpha acid | 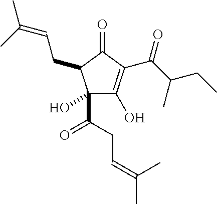 |

In further embodiments of this aspect, the substituted 1,3-cyclopentadione is selected from the group consisting of (4S,5S)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5S)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5S)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5S)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4R,5R)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5R)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5R)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5R)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4S,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4S,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4R,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5R)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4S,5R)-3,4-dihydroxy-4-[(1S)-hydroxy-4-methylpent-3-en-1-yl]-2-(2-methylpropanoyl)-5-(3-methylbut-2-en-1-yl)cyclopent-2-en-1-one; (4S,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4S,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4S,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4R,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4S,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; and to 99.9 percent by weight of (4S,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one.

In another embodiment, the substituted 1,3-cyclopentadione is selected from the group consisting of selected from the group consisting of (4R,5S)-3,4-dihydroxy-2-(3-methylbutanoyl)-5-(3-methylbutyl)-4-(4-methylpentanoyl)cyclopent-2-en-1-one, (4S,5S)-3,4-dihydroxy-2-(3-methylbutanoyl)-5-(3-methylbutyl)-4-(4-methylpentanoyl)cyclopent-2-en-1-one, (4S,5R)-3,4-dihydroxy-2-(3-methylbutanoyl)-5-(3-methylbutyl)-4-(4-methylpentanoyl)cyclopent-2-en-1-one, (4R,5R)-3,4-dihydroxy-2-(3-methylbutanoyl)-5-(3-methylbutyl)-4-(4-methylpentanoyl)cyclopent-2-en-1-one, (4R,5S)-3,4-dihydroxy-5-(3-methylbutyl)-4-(4-methylpentanoyl)-2-(3-methylpropanoyl)cyclopent-2-en-1-one, (4S,5S)-3,4-dihydroxy-5-(3-methylbutyl)-4-(4-methylpentanoyl)-2-(3-methylpropanoyl)cyclopent-2-en-1-one, (4S,5R)-3,4-dihydroxy-5-(3-methylbutyl)-4-(4-methylpentanoyl)-2-(3-methylpropanoyl)cyclopent-2-en-1-one, (4R,5R)-3,4-dihydroxy-5-(3-methylbutyl)-4-(4-methylpentanoyl)-2-(3-methylpropanoyl)cyclopent-2-en-1-one, (4R,5S)-3,4-dihydroxy-2-(2-methylbutanoyl)-5-(3-methylbutyl)-4-(4-methylpentanoyl)cyclopent-2-en-1-one, (4S,5S)-3,4-dihydroxy-2-(2-methylbutanoyl)-5-(3-methylbutyl)-4-(4-methylpentanoyl)cyclopent-2-en-1-one, (4S,5R)-3,4-dihydroxy-2-(2-methylbutanoyl)-5-(3-methylbutyl)-4-(4-methylpentanoyl)cyclopent-2-en-1-one, and (4R,5R)-3,4-dihydroxy-2-(2-methylbutanoyl)-5-(3-methylbutyl)-4-(4-methylpentanoyl)cyclopent-2-en-1-one.

In yet other embodiments, the substituted 1,3-cyclopentadione is selected from the group consisting of selected from the group consisting of (4S,5S)-3,4-dihydroxy-4-[(1S)-1-hydroxy-4-methylpentyl]-2-(3-methylbutanoyl)-5-(3-methylbutyl)cyclopent-2-en-1-one, (4S,5S)-3,4-dihydroxy-4-[(1R)-1-hydroxy-4-methylpentyl]-2-(3-methylbutanoyl)-5-(3-methylbutyl)cyclopent-2-en-1-one, (4R,5S)-3,4-dihydroxy-4-[(1R)-1-hydroxy-4-methylpentyl]-2-(3-methylbutanoyl)-5-(3-methylbutyl)cyclopent-2-en-1-one, (4R,5S)-3,4-dihydroxy-4-[(1S)-1-hydroxy-4-methylpentyl]-2-(3-methylbutanoyl)-5-(3-methylbutyl)cyclopent-2-en-1-one, (4R,5R)-3,4-dihydroxy-4-[(1R)-1-hydroxy-4-methylpentyl]-2-(3-methylbutanoyl)-5-(3-methylbutyl)cyclopent-2-en-1-one, (4R,5R)-3,4-dihydroxy-4-[(1S)-1-hydroxy-4-methylpentyl]-2-(3-methylbutanoyl)-5-(3-methylbutyl)cyclopent-2-en-1-one, (4S,5R)-3,4-dihydroxy-4-[(1S)-1-hydroxy-4-methylpentyl]-2-(3-methylbutanoyl)-5-(3-methylbutyl)cyclopent-2-en-1-one, (4S,5R)-3,4-dihydroxy-4-[(1R)-1-hydroxy-4-methylpentyl]-2-(3-methylbutanoyl)-5-(3-methylbutyl)cyclopent-2-en-1-one, (4S,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4S,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4R,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5R)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4S,5R)-3,4-dihydroxy-4-[(1S)-hydroxy-4-methylpent-3-en-1-yl]-2-(2-methylpropanoyl)-5-(3-methylbut-2-en-1-yl)cyclopent-2-en-1-one, (4S,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4S,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4S,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4R,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4S,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, and (4S,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one.

As used herein, “substituted 1,3-cyclopentadione compounds” refers to those compounds generally described as reduced isoalpha acids commonly associated with hops and beer production. The substituted 1,3-cyclopentadione compounds refers to the dihydroisoalpha acids (RIAA), tetrahydroisoalpha acids (“THIAA”) and hexahydroisalpha acids (“HHIAA”). Examples of reduced isoalpha acids (RIAA) include without limitation dihydroisoalpha acids, more specifically Rho dihydroisoalpha acids (Table 4), tetrahydroisoalpha acid (Table 5), and hexahydroisoalpha acids (Table 6), and their derivatives. “Rho” refers to those reduced isoalpha acids wherein the reduction is a reduction of the carbonyl group in the 4-methyl-3-pentenoyl side chain.” refers to those compounds generally described as reduced isoalpha acids commonly associated with hops and beer production.

TABLE 4
Substituted 1,3-cyclopentadione compounds

| | Syno- | |
|---|---|---|
| Chemical Name | nym | Structure |
| (4S,5S)-3,4-dihydroxy-4- [(1S)-hydroxy-4- methylpent-3-en-1-yl]-2-(3- methylbutanoyl)-5-(3- methylbut-2-en-1- yl)cyclopent-2-en-1-one | rho (6S) cis n iso- alpha acid | 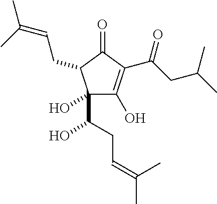 |
| (4S,5S)-3,4-dihydroxy-4- [(1R)-hydroxy-4- methylpent-3-en-1-yl]-2-(3- methylbutanoyl)-5-(3- methylbut-2-en-1- yl)cyclopent-2-en-1-one | rho (6R) cis n iso- alpha acid | 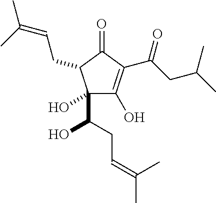 |
| (4R,5S)-3,4-dihydroxy-4- [(1R)-hydroxy-4- methylpent-3-en-1-yl]-2-(3- methylbutanoyl)-5-(3- methylbut-2-en-1- yl)cyclopent-2-en-1-one | rho (6R) trans n iso- alpha acid | 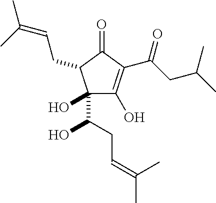 |
| (4R,5S)-3,4-dihydroxy-4- [(1S)-hydroxy-4- methylpent-3-en-1-yl]-2-(3- methylbutanoyl)-5-(3- methylbut-2-en-1- yl)cyclopent-2-en-1-one | rho (6S) trans n iso- alpha acid | 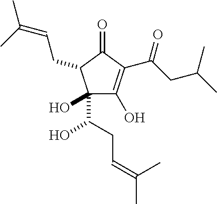 |
| (4R,5R)-3,4-dihydroxy-4- [(1R)-hydroxy-4- methylpent-3-en-1-yl]-2-(3- methylbutanoyl)-5-(3- methylbut-2-en-1- yl)cyclopent-2-en-1-one | rho (6R) cis rho n iso- alpha acid | 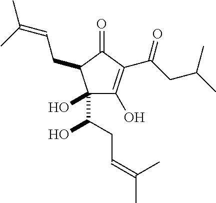 |
| (4R,5R)-3,4-dihydroxy-4- [(1S)-hydroxy-4- methylpent-3-en-1-yl]-2-(3- methylbutanoyl)-5-(3- methylbut-2-en-1- yl)cyclopent-2-en-1-one | rho (6S) cis n iso- alpha acid | 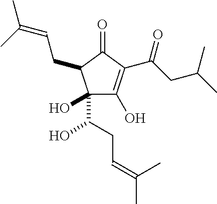 |
| (4S,5R)-3,4-dihydroxy-4- [(1S)-hydroxy-4- methylpent-3-en-1-yl]-2-(3- methylbutanoyl)-5-(3- methylbut-2-en-1- yl)cyclopent-2-en-1-one | (6S) trans rho n iso- alpha acid | 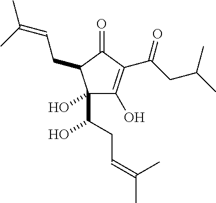 |
| (4S,5R)-3,4-dihydroxy-4- [(1R)-hydroxy-4- methylpent-3-en-1-yl]-2-(3- methylbutanoyl)-5-(3- methylbut-2-en-1- yl)cyclopent-2-en-1-one | rho (6R) trans n iso- alpha acid | 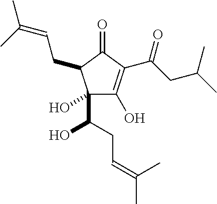 |
| (4S,5S)-3,4-dihydroxy-4- [(1S)-hydroxy-4- methylpent-3-en-1-yl]-5-(3- methylbut-2-en-1-yl)-2-(2- methylpropanoyl)cyclopent- 2-en-1-one | rho (6S) cis co iso- alpha acid | 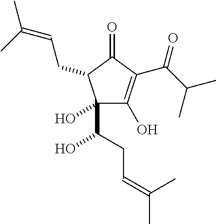 |
| (4S,5S)-3,4-dihydroxy-4- [(1R)-hydroxy-4- methylpent-3-en-1-yl]-5-(3- methylbut-2-en-1-yl)-2-(2- methylpropanoyl)cyclopent- 2-en-1-one | rho (6R) cis co iso- alpha acid | 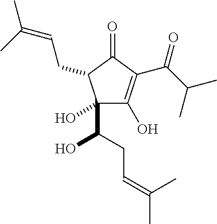 |
| (4R,5S)-3,4-dihydroxy-4- [(1R)-hydroxy-4- methylpent-3-en-1-yl]-5-(3- methylbut-2-en-1-yl)-2-(2- methylpropanoyl)cyclopent- 2-en-1-one | rho (6R) trans co iso- alpha acid | 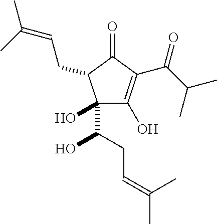 |
| (4R,5S)-3,4-dihydroxy-4- [(1S)-hydroxy-4- methylpent-3-en-1-yl]-5-(3- methylbut-2-en-1-yl)-2-(2- methylpropanoyl)cyclopent- 2-en-1-one | rho (6S) trans co iso- alpha acid | 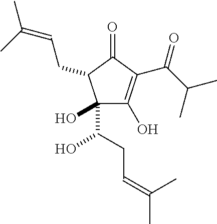 |
| (4R,5R)-3,4-dihydroxy-4- [(1R)-hydroxy-4- methylpent-3-en-1-yl]-5-(3- methylbut-2-en-1-yl)-2-(2- methylpropanoyl)cyclopent- 2-en-1-one | rho (6R) cis co iso- alpha acid | 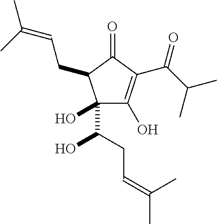 |
| (4R,5R)-3,4-dihydroxy-4- [(1S)-hydroxy-4- methylpent-3-en-1-yl]-5-(3- methylbut-2-en-1-yl)-2-(2- methylpropanoyl)cyclopent- 2-en-1-one | rho (6S) cis co iso- alpha acid | 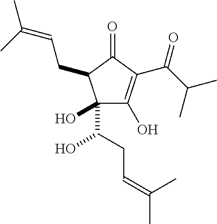 |
| (4S,5R)-3,4-dihydroxy-4- [(1S)-hydroxy-4- methylpent-3-en-1-yl]- 2-(2-methylpropanoyl) 5-(3-methylbut-2- en-1-yl)-cyclopent- 2-en-1-one | rho (6S) trans co iso- alpha acid | 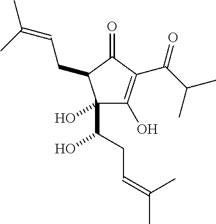 |
| (4S,5R)-3,4-dihydroxy-4- [(1R)-hydroxy-4- methylpent-3-en-1-yl]-5-(3- methylbut-2-en-1-yl)-2-(2- methylpropanoyl)cyclopent- 2-en-1-one | rho (6R) trans co iso- alpha acid | 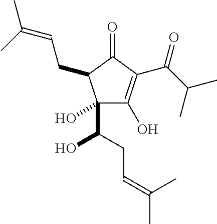 |
| (4S,5S)-3,4-dihydroxy-4- [(1S)-hydroxy-4- methylpent-3-en-1-yl]-2-(2- methylbutanoyl)-5-(3- methylbut-2-en-1- yl)cyclopent-2-en-1-one | rho (6S) cis ad iso- alpha acid | 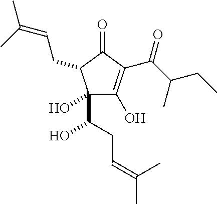 |
| (4S,5S)-3,4-dihydroxy-4- [(1R)-hydroxy-4- methylpent-3-en-1-yl]-2-(2- methylbutanoyl)-5-(3- methylbut-2-en-1- yl)cyclopent-2-en-1-one | rho (6R) cis ad iso- alpha acid | 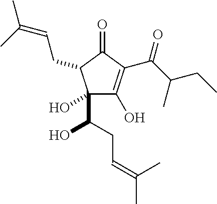 |
| (4R,5S)-3,4-dihydroxy-4- [(1R)-hydroxy-4- methylpent-3-en-1-yl]-2-(2- methylbutanoyl)-5-(3- methylbut-2-en-1- yl)cyclopent-2-en-1-one | rho (6R) trans ad iso- alpha acid | 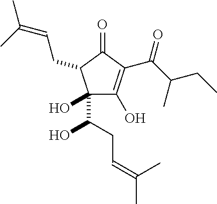 |
| (4R,5S)-3,4-dihydroxy-4- [(1S)-hydroxy-4- methylpent-3-en-1-yl]-2-(2- methylbutanoyl)-5-(3- methylbut-2-en-1- yl)cyclopent-2-en-1-one | rho (6S) trans ad iso- alpha acid | 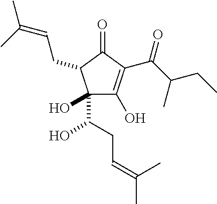 |
| (4R,5R)-3,4-dihydroxy-4- [(1R)-hydroxy-4- methylpent-3-en-1-yl]-2-(2- methylbutanoyl)-5-(3- methylbut-2-en-1- yl)cyclopent-2-en-1-one | rho (6R) cis ad iso- alpha acid | 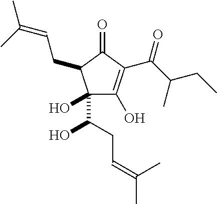 |
| (4R,5R)-3,4-dihydroxy-4- [(1S)-hydroxy-4- methylpent-3-en-1-yl]-2-(2- methylbutanoyl)-5-(3- methylbut-2-en-1- yl)cyclopent-2-en-1-one | rho (6S) cis ad iso- alpha acid | 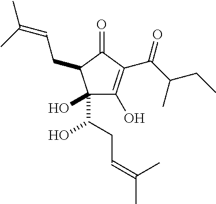 |
| (4S,5R)-3,4-dihydroxy-4- [(1S)-hydroxy-4- methylpent-3-en-1-yl]-2-(2- methylbutanoyl)-5-(3- methylbut-2-en-1- yl)cyclopent-2-en-1-one | rho (6S) trans ad iso- alpha acid | 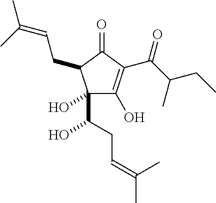 |
| (4S,5R)-3,4-dihydroxy-4- [(1R)-hydroxy-4- methylpent-3-en-1-yl]-2-(2- methylbutanoyl)-5-(3- methylbut-2-en-1- yl)cyclopent-2-en-1-one | rho (6R) trans ad iso- alpha acid | 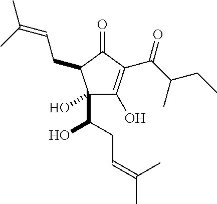 |

TABLE 5
Substituted 1,3-cyclopentadione compounds

| Chemical Name | Synonym | Structure |
|---|---|---|
| (4R,5S)-3,4-dihydroxy-2-(3- methylbutanoyl)-5-(3- methylbutyl)-4-(4- methylpentanoyl)cyclopent- 2-en-1-one | tetrahydro cis n iso- alpha acid | 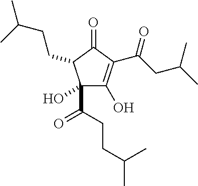 |
| (4S,5S)-3,4-dihydroxy-2-(3- methylbutanoyl)-5-(3- methylbutyl)-4-(4- methylpentanoyl)cyclopent- 2-en-1-one | tetrahydro trans n iso- alpha acid | 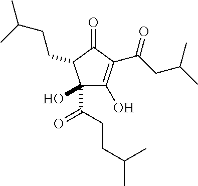 |
| (4S,5R)-3,4-dihydroxy-2-(3- methylbutanoyl)-5-(3- methylbutyl)-4-(4- methylpentanoyl)cyclopent- 2-en-1-one | tetrahydro cis n iso- alpha acid | 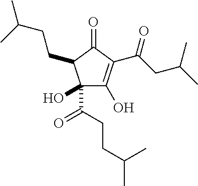 |
| (4R,5R)-3,4-dihydroxy-2-(3- methylbutanoyl)-5-(3- methylbutyl)-4-(4- methylpentanoyl)cyclopent- 2-en-1-one | tetrahydro trans n iso- alpha acid | 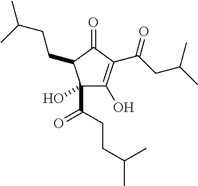 |
| (4R,5S)-3,4-dihydroxy-5-(3- methylbutyl)-4-(4- methylpentanoyl)-2-(3- methylpropanoyl)cyclopent- 2-en-1-one | tetrahydro cis co iso- alpha acid | 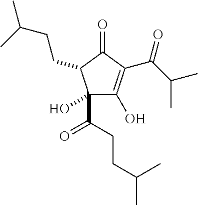 |
| (4S,5S)-3,4-dihydroxy-5-(3- methylbutyl)-4-(4- methylpentanoyl)-2-(3- methylpropanoyl)cyclopent- 2-en-1-one | tetrahydro trans co iso-alpha acid | 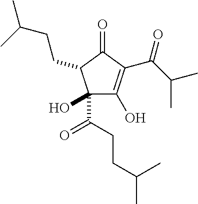 |
| (4S,5R)-3,4-dihydroxy-5-(3- methylbutyl)-4-(4- methylpentanoyl)-2-(3- methylpropanoyl)cyclopent- 2-en-1-one | tetrahydro cis co iso- alpha acid | 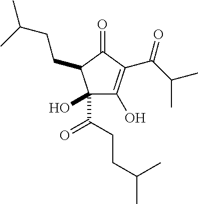 |
| (4R,5R)-3,4-dihydroxy-5-(3- methylbutyl)-4-(4- methylpentanoyl)-2-(3- methylpropanoyl)cyclopent- 2-en-1-one | tetrahydro trans co iso-alpha acid | 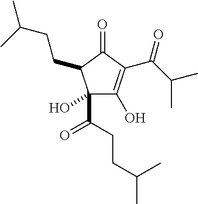 |
| (4R,5S)-3,4-dihydroxy-2-(2- methylbutanoyl)-5-(3- methylbutyl)-4-(4- methylpentanoyl)cyclopent- 2-en-1-one | tetrahydro cis ad iso- alpha acid | 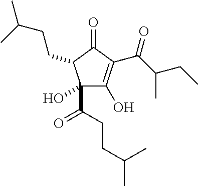 |
| (4S,5S)-3,4-dihydroxy-2-(2- methylbutanoyl)-5-(3- methylbutyl)-4-(4- methylpentanoyl)cyclopent- 2-en-1-one | tetrahydro trans ad iso-alpha acid | 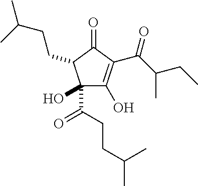 |
| (4S,5R)-3,4-dihydroxy-2-(2- methylbutanoyl)-5-(3- methylbutyl)-4-(4- methylpentanoyl)cyclopent- 2-en-1-one | tetrahydro cis ad iso- alpha acid | 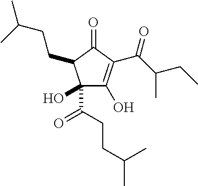 |
| (4R,5R)-3,4-dihydroxy-2-(2- methylbutanoyl)-5-(3- methylbutyl)-4-(4- methylpentanoyl)cyclopent- 2-en-1-one | tetrahydro trans ad iso-alpha acid | 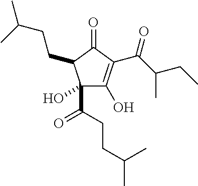 |

TABLE 6
Substituted 1,3-cyclopentadione compounds

| Chemical Name | Synonym | Structure |
|---|---|---|
| (4S,5S)-3,4-dihydroxy-4- [(1S)-1-hydroxy-4- methylpentyl]-2-(3- methylbutanoyl)-5-(3- methylbutyl)cyclopent-2-en- 1-one | hexahydro (6S) cis n iso-alpha acid | 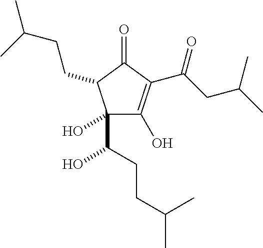 |
| (4S,5S)-3,4-dihydroxy-4- [(1R)-1-hydroxy-4- methylpentyl]-2-(3- methylbutanoyl)-5-(3- methylbutyl)cyclopent-2-en- 1-one | hexahydro (6R) cis n iso-alpha acid | 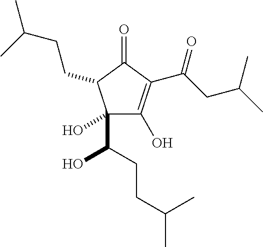 |
| (4R,5S)-3,4-dihydroxy-4- [(1R)-1-hydroxy-4- methylpentyl]-2-(3- methylbutanoyl)-5-(3- methylbutyl)cyclopent-2-en- 1-one | hexahydro (6R) trans n iso-alpha acid | 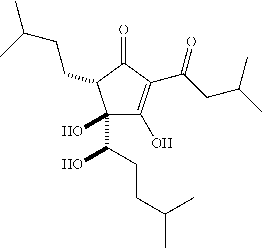 |
| (4R,5S)-3,4-dihydroxy-4- [(1S)-1-hydroxy-4- methylpentyl]-2-(3- methylbutanoyl)-5-(3- methylbutyl)cyclopent-2-en- 1-one | hexahydro (6S) trans n iso-alpha acid | 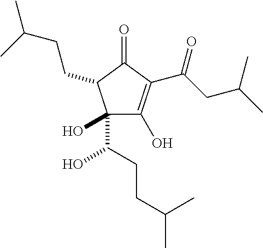 |
| (4R,5R)-3,4-dihydroxy-4- [(1R)-1-hydroxy-4- methylpentyl]-2-(3- methylbutanoyl)-5-(3- methylbutyl)cyclopent-2-en- 1-one | hexahydro (6R) cis n iso-alpha acid | 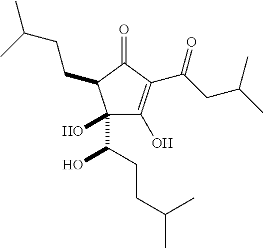 |
| (4R,5R)-3,4-dihydroxy-4- [(1S)-1-hydroxy-4- methylpentyl]-2-(3- methylbutanoyl)-5-(3- methylbutyl)cyclopent-2-en- 1-one | hexahydro (6S) cis n iso-alpha acid | 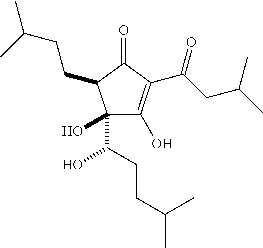 |
| (4S,5R)-3,4-dihydroxy-4- [(1S)-1-hydroxy-4- methylpentyl]-2-(3- methylbutanoyl)-5-(3- methylbutyl)cyclopent-2-en- 1-one | hexahydro (6S) trans n iso-alpha acid | 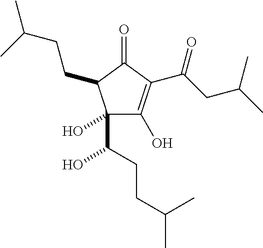 |
| (4S,5R)-3,4-dihydroxy-4- [(1R)-1-hydroxy-4- methylpentyl]-2-(3- methylbutanoyl)-5-(3- methylbutyl)cyclopent-2-en- 1-one | hexahydro (6R) trans n iso-alpha acid | 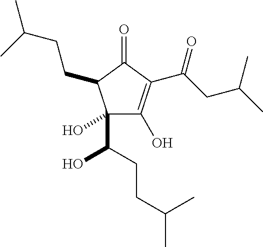 |
| (4S,5S)-3,4-dihydroxy-4- [(1S)-hydroxy-4- methylpent-3-en-1-yl]-5-(3- methylbut-2-en-1-yl)-2-(2- methylpropanoyl)cyclopent- 2-en-1-one | hexahydro (6S) cis co iso-alpha acid | 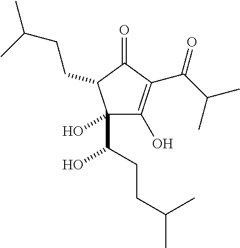 |
| (4S,5S)-3,4-dihydroxy-4- [(1R)-hydroxy-4- methylpent-3-en-1-yl]-5-(3- methylbut-2-en-1-yl)-2-(2- methylpropanoyl)cyclopent- 2-en-1-one | hexahydro (6R) cis co iso-alpha acid | 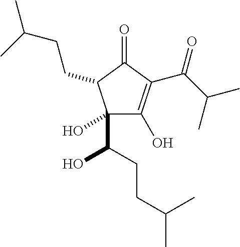 |
| (4R,5S)-3,4-dihydroxy-4- [(1R)-hydroxy-4- methylpent-3-en-1-yl]-5-(3- methylbut-2-en-1-yl)-2-(2- methylpropanoyl)cyclopent- 2-en-1-one | hexahydro (6R) trans co iso- alpha acid | 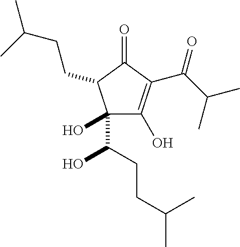 |
| (4R,5S)-3,4-dihydroxy-4- [(1S)-hydroxy-4- methylpent-3-en-1-yl]-5-(3- methylbut-2-en-1-yl)-2-(2- methylpropanoyl)cyclopent- 2-en-1-one | hexahydro (6S) trans co iso- alpha acid | 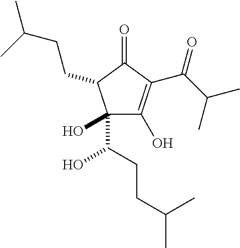 |
| (4R,5R)-3,4-dihydroxy-4- [(1R)-hydroxy-4- methylpent-3-en-1-yl]-5-(3- methylbut-2-en-1-yl)-2-(2- methylpropanoyl)cyclopent- 2-en-1-one | hexahydro (6R) cis co iso-alpha acid | 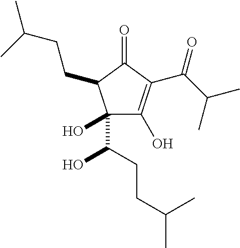 |
| (4R,5R)-3,4-dihydroxy-4- [(1S)-hydroxy-4- methylpent-3-en-1-yl]-5-(3- methylbut-2-en-1-yl)-2-(2- methylpropanoyl)cyclopent- 2-en-1-one | hexahydro (6S) cis co iso-alpha acid | 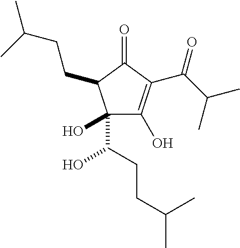 |
| (4S,5R)-3,4-dihydroxy-4- [(1S)-hydroxy-4- methylpent-3-en-1-yl]- 2-(2-methylpropanoyl) 5-(3-methylbut-2- en-1-yl)-cyclopent- 2-en-1-one | hexahydro (6S) trans co iso- alpha acid | 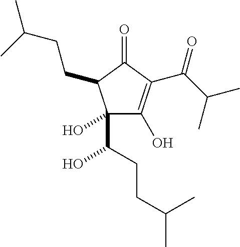 |
| (4S,5R)-3,4-dihydroxy-4- [(1R)-hydroxy-4- methylpent-3-en-1-yl]-5-(3- methylbut-2-en-1-yl)-2-(2- methylpropanoyl)cyclopent- 2-en-1-one | hexahydro (6R) trans co iso- alpha acid | 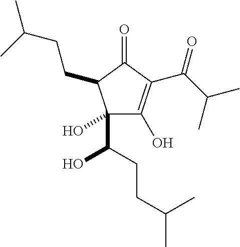 |
| (4S,5S)-3,4-dihydroxy-4- [(1S)-hydroxy-4- methylpent-3-en-1-yl]-2-(2- methylbutanoyl)-5-(3- methylbut-2-en-1- yl)cyclopent-2-en-1-one | hexahydro (6S) cis ad iso-alpha acid | 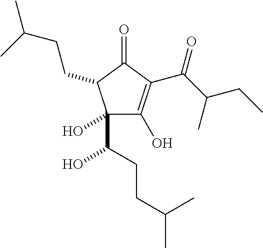 |
| (4S,5S)-3,4-dihydroxy-4- [(1R)-hydroxy-4- methylpent-3-en-1-yl]-2-(2- methylbutanoyl)-5-(3- methylbut-2-en-1- yl)cyclopent-2-en-1-one | hexahydro (6R) cis ad iso-alpha acid | 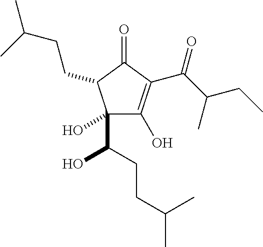 |
| (4R,5S)-3,4-dihydroxy-4- [(1R)-hydroxy-4- methylpent-3-en-1-yl]-2-(2- methylbutanoyl)-5-(3- methylbut-2-en-1- yl)cyclopent-2-en-1-one | hexahydro (6R) trans ad iso- alpha acid | 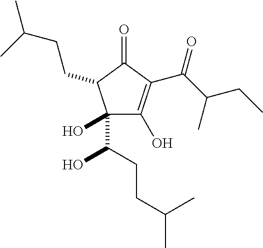 |
| (4R,5S)-3,4-dihydroxy-4- [(1S)-hydroxy-4- methylpent-3-en-1-yl]-2-(2- methylbutanoyl)-5-(3- methylbut-2-en-1- yl)cyclopent-2-en-1-one | hexahydro (6S) trans ad iso- alpha acid | 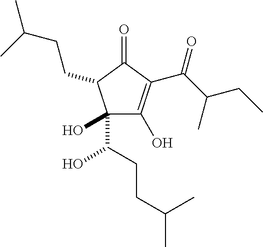 |
| (4R,5R)-3,4-dihydroxy-4- [(1R)-hydroxy-4- methylpent-3-en-1-yl]-2-(2- methylbutanoyl)-5-(3- methylbut-2-en-1- yl)cyclopent-2-en-1-one | hexahydro (6R) cis ad iso-alpha acid | 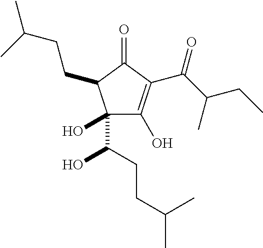 |
| (4R,5R)-3,4-dihydroxy-4- [(1S)-hydroxy-4- methylpent-3-en-1-yl]-2-(2- methylbutanoyl)-5-(3- methylbut-2-en-1- yl)cyclopent-2-en-1-one | hexahydro (6S) cis ad iso-alpha acid | 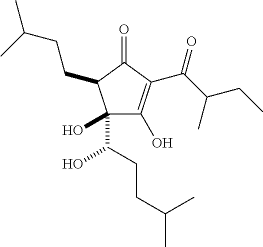 |
| (4S,5R)-3,4-dihydroxy-4- [(1S)-hydroxy-4- methylpent-3-en-1-yl]-2-(2- methylbutanoyl)-5-(3- methylbut-2-en-1- yl)cyclopent-2-en-1-one | hexahydro (6S) trans ad iso- alpha acid | 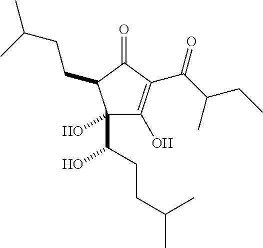 |
| (4S,5R)-3,4-dihydroxy-4- [(1R)-hydroxy-4- methylpent-3-en-1-yl]-2-(2- methylbutanoyl)-5-(3- methylbut-2-en-1- yl)cyclopent-2-en-1-one | hexahydro (6R) trans ad iso- alpha acid | 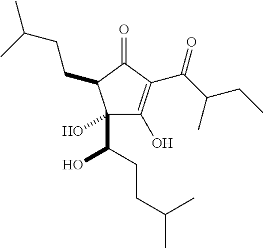 |

In still other embodiments of this aspect, solvent for the bi-phase system may be, for example, water preferably having a pH between 0 and 14, or preferably a pH between 1 and 13, 3 and 12, or 5 and 10; water containing a buffering agent with a pH between 0 and 14, 1 and 13, 3 and 12, or 5 and 10; water containing a soluble polymer; a pentane; hexane; heptane; octane; methyl acetate; ethyl acetate; propyl acetate; butyl acetate; tert-butyl acetate; methanol; ethanol; propanol; iso propanol; butanol; tert butanol; dimethyl formamide; dimethyl sulfoxide; dichloromethane; chloroform; or acetone, or any combination thereof.

In further embodiments, the bi-phasic solvent system has a partition coefficient between 0.6 and 3.0, while in other embodiments the bi-phasic solvent system has a partition coefficient between 0.7 and 1.5.

The counter current chromatography is performed at a temperature of about 20° C. to about 30° C. in some embodiments of the invention while the counter current chromatography may be performed at ambient temperature in other embodiments.

As used herein, “compounds” may be identified either by their chemical structure, chemical name, or common name. When the chemical structure and chemical or common name conflict, the chemical structure is determinative of the identity of the compound. The compounds described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated or identified compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated or identified compounds. The compounds described also encompass isotopically labeled compounds where one or more atoms have an atomic mass different from the atomic mass conventionally found in nature. Examples of isotopes that may be incorporated into the compounds of the invention include, but are not limited to, ²H, ³H, ¹³C, ¹⁴C, ¹⁵N, ¹⁸O, ¹⁷O, etc. Compounds may exist in unsolvated forms as well as solvated forms, including hydrated forms and as N-oxides. In general, compounds may be hydrated, solvated or N-oxides. Certain compounds may exist in multiple crystalline or amorphous forms. Also contemplated within the scope of the invention are congeners, analogs, hydrolysis products, metabolites and precursor or prodrugs of the compound. In general, unless otherwise indicated, all physical forms are equivalent for the uses contemplated herein and are intended to be within the scope of the present invention.

Compounds according to the invention may be present as salts. In particular, pharmaceutically acceptable salts of the compounds are contemplated. A “pharmaceutically acceptable salt” of the invention is a combination of a compound of the invention and either an acid or a base that forms a salt (such as, for example, the magnesium salt, denoted herein as “Mg” or “Mag”) with the compound and is tolerated by a subject under therapeutic conditions. In general, a pharmaceutically acceptable salt of a compound of the invention will have a therapeutic index (the ratio of the lowest toxic dose to the lowest therapeutically effective dose) of 1 or greater. The person skilled in the art will recognize that the lowest therapeutically effective dose will vary from subject to subject and from indication to indication, and will thus adjust accordingly.

A second aspect of the invention discloses compositions comprising a substantially pure congener or a pharmaceutically acceptable salt thereof, wherein the congener is obtained from a mixture of congeners comprising the steps of:
a. selecting a congener to be isolated;
b. dissolving the mixture of congeners in a bi-phasic solvent system specific to the selected congener to be isolated, wherein said bi-phasic solvent system has a partition coefficient from about 0.5 to 5.0;
c. subjecting the mixture of congeners dissolved in the bi-phasic solvent system to a counter current chromatography; and
d. isolating the selected congener;
   where in this aspect the congener is selected from the group consisting of substituted cyclohexa-2,4-dienones, substituted cyclohexane-1,3,5-triones, substituted cyclopent-2-en-1-ones, and substituted 1,3-cyclopentadiones.

In some embodiments of this aspect, the substituted cyclohexa-2,4-dienone is selected from the group consisting of (6R)-3,5,6-trihydroxy-2-(3-methylbutanoyl)-4,6-bis(3-methylbut-2-en-1-yl)cyclohexa-2,4-dien-1-one, (6R)-3,5,6-trihydroxy-4,6-bis(3-methylbut-2-en-1-yl)-2-(2-methylpropanoyl)cyclohexa-2,4-dien-1-one, and (6R)-3,5,6-trihydroxy-2-(2-methylbutanoyl)-4,6-bis(3-methylbut-2-en-1-yl)cyclohexa-2,4-dien-1-one.

In further embodiments, the substituted cyclohexane-1,3,5-trione is selected from the group consisting of substituted cyclohexane-1,3,5-triones wherein said composition is enriched for one or more compounds selected from the group consisting of 3,5-dihydroxy-2-(3-methylbutanoyl)-4,6,6-tris(3-methylbut-2-en-1-yl)cyclohexa-2,4-dien-1-one, 3,5-dihydroxy-4,6,6-tris(3-methylbut-2-en-1-yl)-2-(2-methylpropanoyl)cyclohexa-2,4-dien-1-one, and 3,5-dihydroxy-2-(2-methylbutanoyl)-4,6,6-tris(3-methylbut-2-en-1-yl)cyclohexa-2,4-dien-1-one.

In still other embodiments, the substituted cyclopent-2-en-1-one is selected from the group consisting of (4R,5S)-4-hydroxy-3-methyl-2-(3-methylbutanoyl)-5-(3-methylbut-2-en-1-yl)-4-(4-methylpent-3-enoyl)cyclopent-2-en-1-one, (4S,5S)-4-hydroxy-3-methyl-2-(3-methylbutanoyl)-5-(3-methylbut-2-en-1-yl)-4-(4-methylpent-3-enoyl)cyclopent-2-en-1-one, (4S,5R)-4-hydroxy-3-methyl-2-(3-methylbutanoyl)-5-(3-methylbut-2-en-1-yl)-4-(4-methylpent-3-enoyl)cyclopent-2-en-1-one, (4R,5R)-4-hydroxy-3-methyl-2-(3-methylbutanoyl)-5-(3-methylbut-2-en-1-yl)-4-(4-methylpent-3-enoyl)cyclopent-2-en-1-one, (4R,5S)-4-hydroxy-3-methyl-5-(3-methylbut-2-en-1-yl)-4-(4-methylpent-3-enoyl)-2-(2-methylpropanoyl)cyclopent-2-en-1-one, (4S,5S)-4-hydroxy-3-methyl-5-(3-methylbut-2-en-1-yl)-4-(4-methylpent-3-enoyl)-2-(2-methylpropanoyl)cyclopent-2-en-1-one, (4S,5R)-4-hydroxy-3-methyl-5-(3-methylbut-2-en-1-yl)-4-(4-methylpent-3-enoyl)-2-(2-methylpropanoyl)cyclopent-2-en-1-one, (4R,5R)-4-hydroxy-3-methyl-5-(3-methylbut-2-en-1-yl)-4-(4-methylpent-3-enoyl)-2-(2-methylpropanoyl)cyclopent-2-en-1-one, (4R,5S)-4-hydroxy-3-methyl-2-(2-methylbutanoyl)-5-(3-methylbut-2-en-1-yl)-4-(4-methylpent-3-enoyl)cyclopent-2-en-1-one, (4S,5S)-4-hydroxy-3-methyl-2-(2-methylbutanoyl)-5-(3-methylbut-2-en-1-yl)-4-(4-methylpent-3-enoyl)cyclopent-2-en-1-one, (4S,5R)-4-hydroxy-3-methyl-2-(2-methylbutanoyl)-5-(3-methylbut-2-en-1-yl)-4-(4-methylpent-3-enoyl)cyclopent-2-en-1-one, and (4R,5R)-4-hydroxy-3-methyl-2-(2-methylbutanoyl)-5-(3-methylbut-2-en-1-yl)-4-(4-methylpent-3-enoyl)cyclopent-2-en-1-one.

In further embodiments of this aspect, the substituted 1,3-cyclopentadione is selected from the group consisting of (4S,5S)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5S)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5S)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5S)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4R,5R)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5R)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5R)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5R)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4S,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4S,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4R,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5R)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4S,5R)-3,4-dihydroxy-4-[(1S)-hydroxy-4-methylpent-3-en-1-yl]-2-(2-methylpropanoyl)-5-(3-methylbut-2-en-1-yl)cyclopent-2-en-1-one; (4S,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4S,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4S,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4R,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4S,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; and to 99.9 percent by weight of (4S,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one.

In another embodiment, the substituted 1,3-cyclopentadione is selected from the group consisting of selected from the group consisting of (4R,5S)-3,4-dihydroxy-2-(3-methylbutanoyl)-5-(3-methylbutyl)-4-(4-methylpentanoyl)cyclopent-2-en-1-one, (4S,5S)-3,4-dihydroxy-2-(3-methylbutanoyl)-5-(3-methylbutyl)-4-(4-methylpentanoyl)cyclopent-2-en-1-one, (4S,5R)-3,4-dihydroxy-2-(3-methylbutanoyl)-5-(3-methylbutyl)-4-(4-methylpentanoyl)cyclopent-2-en-1-one, (4R,5R)-3,4-dihydroxy-2-(3-methylbutanoyl)-5-(3-methylbutyl)-4-(4-methylpentanoyl)cyclopent-2-en-1-one, (4R,5S)-3,4-dihydroxy-5-(3-methylbutyl)-4-(4-methylpentanoyl)-2-(3-methylpropanoyl)cyclopent-2-en-1-one, (4S,5S)-3,4-dihydroxy-5-(3-methylbutyl)-4-(4-methylpentanoyl)-2-(3-methylpropanoyl)cyclopent-2-en-1-one, (4S,5R)-3,4-dihydroxy-5-(3-methylbutyl)-4-(4-methylpentanoyl)-2-(3-methylpropanoyl)cyclopent-2-en-1-one, (4R,5R)-3,4-dihydroxy-5-(3-methylbutyl)-4-(4-methylpentanoyl)-2-(3-methylpropanoyl)cyclopent-2-en-1-one, (4R,5S)-3,4-dihydroxy-2-(2-methylbutanoyl)-5-(3-methylbutyl)-4-(4-methylpentanoyl)cyclopent-2-en-1-one, (4S,5S)-3,4-dihydroxy-2-(2-methylbutanoyl)-5-(3-methylbutyl)-4-(4-methylpentanoyl)cyclopent-2-en-1-one, (4S,5R)-3,4-dihydroxy-2-(2-methylbutanoyl)-5-(3-methylbutyl)-4-(4-methylpentanoyl)cyclopent-2-en-1-one, and (4R,5R)-3,4-dihydroxy-2-(2-methylbutanoyl)-5-(3-methylbutyl)-4-(4-methylpentanoyl)cyclopent-2-en-1-one.

In yet other embodiments, the substituted 1,3-cyclopentadione is selected from the group consisting of selected from the group consisting of (4S,5S)-3,4-dihydroxy-4-[(1S)-1-hydroxy-4-methylpentyl]-2-(3-methylbutanoyl)-5-(3-methylbutyl)cyclopent-2-en-1-one, (4S,5S)-3,4-dihydroxy-4-[(1R)-1-hydroxy-4-methylpentyl]-2-(3-methylbutanoyl)-5-(3-methylbutyl)cyclopent-2-en-1-one, (4R,5S)-3,4-dihydroxy-4-[(1R)-1-hydroxy-4-methylpentyl]-2-(3-methylbutanoyl)-5-(3-methylbutyl)cyclopent-2-en-1-one, (4R,5S)-3,4-dihydroxy-4-[(1S)-1-hydroxy-4-methylpentyl]-2-(3-methylbutanoyl)-5-(3-methylbutyl)cyclopent-2-en-1-one, (4R,5R)-3,4-dihydroxy-4-[(1R)-1-hydroxy-4-methylpentyl]-2-(3-methylbutanoyl)-5-(3-methylbutyl)cyclopent-2-en-1-one, (4R,5R)-3,4-dihydroxy-4-[(1S)-1-hydroxy-4-methylpentyl]-2-(3-methylbutanoyl)-5-(3-methylbutyl)cyclopent-2-en-1-one, (4S,5R)-3,4-dihydroxy-4-[(1S)-1-hydroxy-4-methylpentyl]-2-(3-methylbutanoyl)-5-(3-methylbutyl)cyclopent-2-en-1-one, (4S,5R)-3,4-dihydroxy-4-[(1R)-1-hydroxy-4-methylpentyl]-2-(3-methylbutanoyl)-5-(3-methylbutyl)cyclopent-2-en-1-one, (4S,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4S,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4R,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5R)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4S,5R)-3,4-dihydroxy-4-[(1S)-hydroxy-4-methylpent-3-en-1-yl]-2-(2-methylpropanoyl)-5-(3-methylbut-2-en-1-yl)cyclopent-2-en-1-one, (4S,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4S,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4S,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4R,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4S,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, and (4S,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one.

In still other embodiments of this aspect, the solvent for the bi-phasic system is selected from the group comprising water, water containing a buffering agent, water containing a soluble polymer, a pentane, hexane, heptane, octane, methyl acetate, ethyl acetate, propyl acetate, butyl acetate, tert-butyl acetate, methanol, ethanol, propanol, iso propanol, butanol, tert butanol, dimethyl formamide, dimethyl sulfoxide, dichloromethane, chloroform; and acetone, or any combination thereof.

In further embodiments, the bi-phasic solvent system has a partition coefficient between 0.6 and 3.0, while in other embodiments the bi-phasic solvent system has a partition coefficient between 0.7 and 1.5.

The counter current chromatography is performed at a temperature of about 20° C. to about 30° C. in some embodiments of the invention while the counter current chromatography may be performed at ambient temperature in other embodiments.

In some embodiments, the pharmaceutically acceptable excipient is selected from the group consisting of an isotonic and absorption delaying agent, binder, adhesive, lubricant, disintegrant, coloring agent, flavoring agent, sweetening agent, absorbants, detergent, and emulsifying agent, or any combination thereof, while in yet other embodiments, the composition further comprises one or more antioxidants, vitamins, minerals, proteins, fats, and carbohydrates, while in yet other embodiments.

Examples of useful excipients include, but are not limited to, lactose, sucrose, D-mannitol, starch, corn starch, crystalline cellulose, light anhydrous silicic acid and the like. Examples of useful lubricants include, but are not limited to, magnesium stearate, calcium stearate, talc, colloidal silica and the like. Examples of useful binders include, but are not limited to, crystalline cellulose, sucrose, D-mannitol, dextrin, hydroxypropylcellulose, hydroxypropylmethylcllulose, polyvinylpyrrolidone, starch, sucrose, gelatin, methylcellulose, carboxymethylcellulose sodium and the like. Examples of useful disintegrating agents include starch, carboxymethylcellulose, carboxymethylcellulose calcium, carboxymethylstarch sodium, L-hydroxypropylcellulose and the like. Examples of useful solvents include injection water, alcohol, propylene glycol, macrogol, sesame oil, corn oil, olive oil and the like. Examples of useful dissolution aid are polyethylene glycol, propylene glycol, D-mannitol, benzyl benzoate, ethanol, trisaminomethane, cholesterol, triethanolamine, sodium carbonate, sodium citrate and the like. Examples of useful suspending agent are surfactants such as stearyl triethanolamine, sodium laurylsulfate, laurylaminopropionic acid, lecithine, benzalkonium chloride, benzetonium chloride, glycerin monostearate and the like; hydrophilic polymers such as polyvinyl alcohol, polyvinyl pyrrolidone, carboxymethylcellulose sodium, methylcellulose, hydroxymethylcellulose, hydroxyethylcellulose, hydroxypropylcellulose and the like. Examples of useful isotonizing agents include, but are not limited to, glucose, D-sorbitol, sodium chloride, glycerin, D-mannitol and the like. Examples of useful buffers include, but are not limited to, buffer solutions of a phosphate, acetate, carbonate, citrate and the like, etc., are listed. Examples of useful soothing agents include, but are not limited to, benzyl alcohol and the like. Examples of the preservative include p-oxybenzoates, chlorobutanol, benzyl alcohol, phenetyl alcohol, dehydroacetic acid, sorbic acid and the like. Examples of the antioxidant include sulfites, ascorbic acid, α-tocopherol and the like.

The compounds according to the invention are optionally formulated in a pharmaceutically acceptable vehicle with any of the well known pharmaceutically acceptable carriers, including diluents and excipients (see Remington's Pharmaceutical Sciences, 18th Ed., Gennaro, Mack Publishing Co., Easton, Pa. 1990 and Remington: The Science and Practice of Pharmacy, Lippincott, Williams & Wilkins, 1995). While the type of pharmaceutically acceptable carrier/vehicle employed in generating the compositions of the invention will vary depending upon the mode of administration of the composition to a mammal, generally pharmaceutically acceptable carriers are physiologically inert and non-toxic. Formulations of compositions according to the invention may contain more than one type of compound of the invention), as well any other pharmacologically active ingredient useful for the treatment of the symptom/condition being treated.

In further embodiments the composition is in a dosage form suitable for administration via a route selected from the group consisting of oral, inhalation, rectal, ophthalmic, nasal, topical, vaginal, and parenteral.

The formulations of the compositions of the invention may conveniently be presented in unit dosage form and may be prepared by conventional pharmaceutical techniques as discussed above. Such techniques include the step of bringing into association the compound of the invention and the pharmaceutically acceptable carrier(s), such as a diluent or an excipient. In general, the formulations are prepared by uniformly and intimately bringing into association the active ingredient with liquid carriers or finely divided solid carriers or both, and then, if necessary, shaping the product.

The compounding ratio of the compound of the present invention to a combination drug in the present invention can be appropriately selected depending on an administration subject, administration route, diseases and the like. For example, the amount of the reduced isoalpha acids isolated by the method of the present invention can depend on the form of a preparation, and usually be from about 0.01 to 100% by weight, preferably from about 0.1 to 50% by weight, further preferably from about 0.5 to 20% by weight of the composition.

In the case of a preparation for oral administration, an excipient (e.g., lactose, sucrose, starch and the like), a disintegrating agent (e.g., starch, calcium carbonate and the like), a binder (e.g., starch, gum Arabic, carboxymethylcellulose, polyvinylpyrrolidone, hydroxypropylcellulose and the like), a lubricant (e.g., talc, magnesium stearate, polyethylene glycol 6000 and the like) and the like, for example, can be added to the compound of the present invention or a combination drug, according to a method known per se, and the mixture can be compression-molded, then if desirable, the molder product can be coated by a method known per se for the purpose of masking of taste, enteric property or durability, to obtain a preparation for oral administration. As this coating agent, for example, hydroxypropylmethylcellulose, ethylcellulose, hydroxymethylcellulose, hydroxypropylcellulose, polyoxyethylene glycol, Tween 80, Pluronic F68, cellulose acetate phthalate, hydroxypropylmethylcellulose phthalate, hydroxymethylcellulose acetate succinate, Eudoragit (methacrylic acid acrylic acid copolymer, manufactured by Rohm, DE), pigment (e.g., iron oxide red, titanium dioxide, et.) and the like can be used. The preparation for oral administration may be any of a quick release preparation and a sustained release preparation.

For example, in the case of a suppository, the compound of the present invention and a combination drug can be made into an oily or aqueous solid, semisolid or liquid suppository according to methods known in the act. As the oily substrate used in the above-mentioned composition, for example, glycerides of higher fatty acids [e.g., cacao butter, Witebsols (manufactured by Dynamite Novel, DE), etc.], intermediate grade fatty acids [e.g., Myglyols (manufactured by Dynamite Novel, DE), etc.], or vegetable oils (e.g., sesame oil, soy bean oil, cotton seed oil and the like), and the like are listed. Further, as the aqueous substrate, for example, polyethylene glycols, propylene glycol are listed, and as the aqueous gel substrate, for example, natural gums, cellulose derivatives, vinyl polymers, acrylic acid polymers and the like are listed.

An example of a sustained release agent includes, but is not limited to, sustained release microcapsules. For obtaining a sustained release microcapsule, methods known in the act can be adopted, and for example, it is preferably molded into a sustained release preparation shown in section (2) below, before administration.

The compound of the present invention is preferably molded into an oral administration preparation such as a solid preparation (e.g., powder, granule, tablet, capsule) and the like, or molded into a rectum administration preparation such as a suppository. Particularly, an oral administration preparation is preferable.

In another embodiment, the congener in the composition is at least eighty percent pure, while in other embodiments the congener is at least ninety-five percent pure.

A third aspect discloses a method for isolating a congener in substantially pure form from a mixture of congeners comprising the steps of:
a. preparing a suitable mixture of individual congeners derived from an extract of hops;
b. dissolving said mixture with a suitable solvent that can be introduced into a counter current separation instrument for the purpose of purification;
c. collecting a homogenous or partially homogenous solution of individual congeners;
d. extracting the counter current separation purified congeners into a suitable solvent obtained from step (c); or
e. removing the solvent of the solution obtained in step (c) of a homogenous or partially homogenous solution of an individual congener to render the pure or partially pure congener; and
f. removing the solvent of the solution obtained in step (d) of a homogenous or partially homogenous solution of an individual congener to render the pure or partially pure congener;
   wherein said congener is selected from the group consisting of substituted cyclohexa-2,4-dienones, substituted cyclohexane-1,3,5-triones, substituted cyclopent-2-en-1-ones, and substituted 1,3-cyclopentadiones

In some embodiments of this aspect, the substituted cyclohexa-2,4-dienone is selected from the group consisting of (6R)-3,5,6-trihydroxy-2-(3-methylbutanoyl)-4,6-bis(3-methylbut-2-en-1-yl)cyclohexa-2,4-dien-1-one, (6R)-3,5,6-trihydroxy-4,6-bis(3-methylbut-2-en-1-yl)-2-(2-methylpropanoyl)cyclohexa-2,4-dien-1-one, and (6R)-3,5,6-trihydroxy-2-(2-methylbutanoyl)-4,6-bis(3-methylbut-2-en-1-yl)cyclohexa-2,4-dien-1-one.

In further embodiments, the substituted cyclohexane-1,3,5-trione is selected from the group consisting of substituted cyclohexane-1,3,5-triones wherein said composition is enriched for one or more compounds selected from the group consisting of 3,5-dihydroxy-2-(3-methylbutanoyl)-4,6,6-tris(3-methylbut-2-en-1-yl)cyclohexa-2,4-dien-1-one, 3,5-dihydroxy-4,6,6-tris(3-methylbut-2-en-1-yl)-2-(2-methylpropanoyl)cyclohexa-2,4-dien-1-one, and 3,5-dihydroxy-2-(2-methylbutanoyl)-4,6,6-tris(3-methylbut-2-en-1-yl)cyclohexa-2,4-dien-1-one.

In still other embodiments, the substituted cyclopent-2-en-1-one is selected from the group consisting of (4R,5S)-4-hydroxy-3-methyl-2-(3-methylbutanoyl)-5-(3-methylbut-2-en-1-yl)-4-(4-methylpent-3-enoyl)cyclopent-2-en-1-one, (4S,5S)-4-hydroxy-3-methyl-2-(3-methylbutanoyl)-5-(3-methylbut-2-en-1-yl)-4-(4-methylpent-3-enoyl)cyclopent-2-en-1-one, (4S,5R)-4-hydroxy-3-methyl-2-(3-methylbutanoyl)-5-(3-methylbut-2-en-1-yl)-4-(4-methylpent-3-enoyl)cyclopent-2-en-1-one, (4R,5R)-4-hydroxy-3-methyl-2-(3-methylbutanoyl)-5-(3-methylbut-2-en-1-yl)-4-(4-methylpent-3-enoyl)cyclopent-2-en-1-one, (4R,5S)-4-hydroxy-3-methyl-5-(3-methylbut-2-en-1-yl)-4-(4-methylpent-3-enoyl)-2-(2-methylpropanoyl)cyclopent-2-en-1-one, (4S,5S)-4-hydroxy-3-methyl-5-(3-methylbut-2-en-1-yl)-4-(4-methylpent-3-enoyl)-2-(2-methylpropanoyl)cyclopent-2-en-1-one, (4S,5R)-4-hydroxy-3-methyl-5-(3-methylbut-2-en-1-yl)-4-(4-methylpent-3-enoyl)-2-(2-methylpropanoyl)cyclopent-2-en-1-one, (4R,5R)-4-hydroxy-3-methyl-5-(3-methylbut-2-en-1-yl)-4-(4-methylpent-3-enoyl)-2-(2-methylpropanoyl)cyclopent-2-en-1-one, (4R,5S)-4-hydroxy-3-methyl-2-(2-methylbutanoyl)-5-(3-methylbut-2-en-1-yl)-4-(4-methylpent-3-enoyl)cyclopent-2-en-1-one, (4S,5S)-4-hydroxy-3-methyl-2-(2-methylbutanoyl)-5-(3-methylbut-2-en-1-yl)-4-(4-methylpent-3-enoyl)cyclopent-2-en-1-one, (4S,5R)-4-hydroxy-3-methyl-2-(2-methylbutanoyl)-5-(3-methylbut-2-en-1-yl)-4-(4-methylpent-3-enoyl)cyclopent-2-en-1-one, and (4R,5R)-4-hydroxy-3-methyl-2-(2-methylbutanoyl)-5-(3-methylbut-2-en-1-yl)-4-(4-methylpent-3-enoyl)cyclopent-2-en-1-one.

In further embodiments of this aspect, the substituted 1,3-cyclopentadione is selected from the group consisting of (4S,5S)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5S)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5S)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5S)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4R,5R)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5R)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5R)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5R)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4S,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4S,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4R,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5R)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4S,5R)-3,4-dihydroxy-4-[(1S)-hydroxy-4-methylpent-3-en-1-yl]-2-(2-methylpropanoyl)-5-(3-methylbut-2-en-1-yl)cyclopent-2-en-1-one; (4S,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4S,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4S,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4R,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4S,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; and to 99.9 percent by weight of (4S,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one.

In another embodiment, the substituted 1,3-cyclopentadione is selected from the group consisting of selected from the group consisting of (4R,5S)-3,4-dihydroxy-2-(3-methylbutanoyl)-5-(3-methylbutyl)-4-(4-methylpentanoyl)cyclopent-2-en-1-one, (4S,5S)-3,4-dihydroxy-2-(3-methylbutanoyl)-5-(3-methylbutyl)-4-(4-methylpentanoyl)cyclopent-2-en-1-one, (4S,5R)-3,4-dihydroxy-2-(3-methylbutanoyl)-5-(3-methylbutyl)-4-(4-methylpentanoyl)cyclopent-2-en-1-one, (4R,5R)-3,4-dihydroxy-2-(3-methylbutanoyl)-5-(3-methylbutyl)-4-(4-methylpentanoyl)cyclopent-2-en-1-one, (4R,5S)-3,4-dihydroxy-5-(3-methylbutyl)-4-(4-methylpentanoyl)-2-(3-methylpropanoyl)cyclopent-2-en-1-one, (4S,5S)-3,4-dihydroxy-5-(3-methylbutyl)-4-(4-methylpentanoyl)-2-(3-methylpropanoyl)cyclopent-2-en-1-one, (4S,5R)-3,4-dihydroxy-5-(3-methylbutyl)-4-(4-methylpentanoyl)-2-(3-methylpropanoyl)cyclopent-2-en-1-one, (4R,5R)-3,4-dihydroxy-5-(3-methylbutyl)-4-(4-methylpentanoyl)-2-(3-methylpropanoyl)cyclopent-2-en-1-one, (4R,5S)-3,4-dihydroxy-2-(2-methylbutanoyl)-5-(3-methylbutyl)-4-(4-methylpentanoyl)cyclopent-2-en-1-one, (4S,5S)-3,4-dihydroxy-2-(2-methylbutanoyl)-5-(3-methylbutyl)-4-(4-methylpentanoyl)cyclopent-2-en-1-one, (4S,5R)-3,4-dihydroxy-2-(2-methylbutanoyl)-5-(3-methylbutyl)-4-(4-methylpentanoyl)cyclopent-2-en-1-one, and (4R,5R)-3,4-dihydroxy-2-(2-methylbutanoyl)-5-(3-methylbutyl)-4-(4-methylpentanoyl)cyclopent-2-en-1-one.

In yet other embodiments, the substituted 1,3-cyclopentadione is selected from the group consisting of selected from the group consisting of (4S,5S)-3,4-dihydroxy-4-[(1S)-1-hydroxy-4-methylpentyl]-2-(3-methylbutanoyl)-5-(3-methylbutyl)cyclopent-2-en-1-one, (4S,5S)-3,4-dihydroxy-4-[(1R)-1-hydroxy-4-methylpentyl]-2-(3-methylbutanoyl)-5-(3-methylbutyl)cyclopent-2-en-1-one, (4R,5S)-3,4-dihydroxy-4-[(1R)-1-hydroxy-4-methylpentyl]-2-(3-methylbutanoyl)-5-(3-methylbutyl)cyclopent-2-en-1-one, (4R,5S)-3,4-dihydroxy-4-[(1S)-1-hydroxy-4-methylpentyl]-2-(3-methylbutanoyl)-5-(3-methylbutyl)cyclopent-2-en-1-one, (4R,5R)-3,4-dihydroxy-4-[(1R)-1-hydroxy-4-methylpentyl]-2-(3-methylbutanoyl)-5-(3-methylbutyl)cyclopent-2-en-1-one, (4R,5R)-3,4-dihydroxy-4-[(1S)-1-hydroxy-4-methylpentyl]-2-(3-methylbutanoyl)-5-(3-methylbutyl)cyclopent-2-en-1-one, (4S,5R)-3,4-dihydroxy-4-[(1S)-1-hydroxy-4-methylpentyl]-2-(3-methylbutanoyl)-5-(3-methylbutyl)cyclopent-2-en-1-one, (4S,5R)-3,4-dihydroxy-4-[(1R)-1-hydroxy-4-methylpentyl]-2-(3-methylbutanoyl)-5-(3-methylbutyl)cyclopent-2-en-1-one, (4S,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4S,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4R,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5R)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4S,5R)-3,4-dihydroxy-4-[(1S)-hydroxy-4-methylpent-3-en-1-yl]-2-(2-methylpropanoyl)-5-(3-methylbut-2-en-1-yl)cyclopent-2-en-1-one, (4S,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4S,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4S,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4R,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4S,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, and (4S,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one.

In still other embodiments of this aspect, the solvent for the bi-phasic system is selected from the group comprising water, water containing a buffering agent, water containing a soluble polymer, a pentane, hexane, heptane, octane, methyl acetate, ethyl acetate, propyl acetate, butyl acetate, tert-butyl acetate, methanol, ethanol, propanol, iso propanol, butanol, tert butanol, dimethyl formamide, dimethyl sulfoxide, dichloromethane, chloroform; and acetone, or any combination thereof.

In further embodiments, the bi-phasic solvent system has a partition coefficient between 0.6 and 3.0, while in other embodiments the bi-phasic solvent system has a partition coefficient between 0.7 and 1.5.

The counter current chromatography is performed at a temperature of about 20° C. to about 30° C. in some embodiments of the invention while the counter current chromatography may be performed at ambient temperature in other embodiments.

A fourth aspect discloses compositions comprising a substantially pure congener or a pharmaceutically acceptable salt thereof, wherein the congener is obtained from a mixture of congeners comprising the steps of:
a. preparing a suitable mixture of individual congeners derived from an extract of hops;
b. dissolving said mixture with a suitable solvent that can be introduced into a counter current separation instrument for the purpose of purification;
c. collecting a homogenous or partially homogenous solution of individual congeners;
d. extracting the counter current separation purified congeners into a suitable solvent obtained from step (c); or
e. removing the solvent of the solution obtained in step (c) of a homogenous or partially homogenous solution of an individual congener to render the pure or partially pure congener; and
f. removing the solvent of the solution obtained in step (d) of a homogenous or partially homogenous solution of an individual congener to render the pure or partially pure congener;
   wherein said congener is selected from the group consisting of substituted cyclohexa-2,4-dienones, substituted cyclohexane-1,3,5-triones, substituted cyclopent-2-en-1-ones, and substituted 1,3-cyclopentadiones.

In some embodiments of this aspect, the substituted cyclohexa-2,4-dienone is selected from the group consisting of (6R)-3,5,6-trihydroxy-2-(3-methylbutanoyl)-4,6-bis(3-methylbut-2-en-1-yl)cyclohexa-2,4-dien-1-one, (6R)-3,5,6-trihydroxy-4,6-bis(3-methylbut-2-en-1-yl)-2-(2-methylpropanoyl)cyclohexa-2,4-dien-1-one, and (6R)-3,5,6-trihydroxy-2-(2-methylbutanoyl)-4,6-bis(3-methylbut-2-en-1-yl)cyclohexa-2,4-dien-1-one.

In further embodiments, the substituted cyclohexane-1,3,5-trione is selected from the group consisting of substituted cyclohexane-1,3,5-triones wherein said composition is enriched for one or more compounds selected from the group consisting of 3,5-dihydroxy-2-(3-methylbutanoyl)-4,6,6-tris(3-methylbut-2-en-1-yl)cyclohexa-2,4-dien-1-one, 3,5-dihydroxy-4,6,6-tris(3-methylbut-2-en-1-yl)-2-(2-methylpropanoyl)cyclohexa-2,4-dien-1-one, and 3,5-dihydroxy-2-(2-methylbutanoyl)-4,6,6-tris(3-methylbut-2-en-1-yl)cyclohexa-2,4-dien-1-one.

In still other embodiments, the substituted cyclopent-2-en-1-one is selected from the group consisting of (4R,5S)-4-hydroxy-3-methyl-2-(3-methylbutanoyl)-5-(3-methylbut-2-en-1-yl)-4-(4-methylpent-3-enoyl)cyclopent-2-en-1-one, (4S,5S)-4-hydroxy-3-methyl-2-(3-methylbutanoyl)-5-(3-methylbut-2-en-1-yl)-4-(4-methylpent-3-enoyl)cyclopent-2-en-1-one, (4S,5R)-4-hydroxy-3-methyl-2-(3-methylbutanoyl)-5-(3-methylbut-2-en-1-yl)-4-(4-methylpent-3-enoyl)cyclopent-2-en-1-one, (4R,5R)-4-hydroxy-3-methyl-2-(3-methylbutanoyl)-5-(3-methylbut-2-en-1-yl)-4-(4-methylpent-3-enoyl)cyclopent-2-en-1-one, (4R,5S)-4-hydroxy-3-methyl-5-(3-methylbut-2-en-1-yl)-4-(4-methylpent-3-enoyl)-2-(2-methylpropanoyl)cyclopent-2-en-1-one, (4S,5S)-4-hydroxy-3-methyl-5-(3-methylbut-2-en-1-yl)-4-(4-methylpent-3-enoyl)-2-(2-methylpropanoyl)cyclopent-2-en-1-one, (4S,5R)-4-hydroxy-3-methyl-5-(3-methylbut-2-en-1-yl)-4-(4-methylpent-3-enoyl)-2-(2-methylpropanoyl)cyclopent-2-en-1-one, (4R,5R)-4-hydroxy-3-methyl-5-(3-methylbut-2-en-1-yl)-4-(4-methylpent-3-enoyl)-2-(2-methylpropanoyl)cyclopent-2-en-1-one, (4R,5S)-4-hydroxy-3-methyl-2-(2-methylbutanoyl)-5-(3-methylbut-2-en-1-yl)-4-(4-methylpent-3-enoyl)cyclopent-2-en-1-one, (4S,5S)-4-hydroxy-3-methyl-2-(2-methylbutanoyl)-5-(3-methylbut-2-en-1-yl)-4-(4-methylpent-3-enoyl)cyclopent-2-en-1-one, (4S,5R)-4-hydroxy-3-methyl-2-(2-methylbutanoyl)-5-(3-methylbut-2-en-1-yl)-4-(4-methylpent-3-enoyl)cyclopent-2-en-1-one, and (4R,5R)-4-hydroxy-3-methyl-2-(2-methylbutanoyl)-5-(3-methylbut-2-en-1-yl)-4-(4-methylpent-3-enoyl)cyclopent-2-en-1-one.

In further embodiments of this aspect, the substituted 1,3-cyclopentadione is selected from the group consisting of (4S,5S)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5S)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5S)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5S)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4R,5R)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5R)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5R)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5R)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4S,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4S,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4R,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5R)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4S,5R)-3,4-dihydroxy-4-[(1S)-hydroxy-4-methylpent-3-en-1-yl]-2-(2-methylpropanoyl)-5-(3-methylbut-2-en-1-yl)cyclopent-2-en-1-one; (4S,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4S,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4S,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4R,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4S,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; and to 99.9 percent by weight of (4S,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one.

In another embodiment, the substituted 1,3-cyclopentadione is selected from the group consisting of selected from the group consisting of (4R,5S)-3,4-dihydroxy-2-(3-methylbutanoyl)-5-(3-methylbutyl)-4-(4-methylpentanoyl)cyclopent-2-en-1-one, (4S,5S)-3,4-dihydroxy-2-(3-methylbutanoyl)-5-(3-methylbutyl)-4-(4-methylpentanoyl)cyclopent-2-en-1-one, (4S,5R)-3,4-dihydroxy-2-(3-methylbutanoyl)-5-(3-methylbutyl)-4-(4-methylpentanoyl)cyclopent-2-en-1-one, (4R,5R)-3,4-dihydroxy-2-(3-methylbutanoyl)-5-(3-methylbutyl)-4-(4-methylpentanoyl)cyclopent-2-en-1-one, (4R,5S)-3,4-dihydroxy-5-(3-methylbutyl)-4-(4-methylpentanoyl)-2-(3-methylpropanoyl)cyclopent-2-en-1-one, (4S,5S)-3,4-dihydroxy-5-(3-methylbutyl)-4-(4-methylpentanoyl)-2-(3-methylpropanoyl)cyclopent-2-en-1-one, (4S,5R)-3,4-dihydroxy-5-(3-methylbutyl)-4-(4-methylpentanoyl)-2-(3-methylpropanoyl)cyclopent-2-en-1-one, (4R,5R)-3,4-dihydroxy-5-(3-methylbutyl)-4-(4-methylpentanoyl)-2-(3-methylpropanoyl)cyclopent-2-en-1-one, (4R,5S)-3,4-dihydroxy-2-(2-methylbutanoyl)-5-(3-methylbutyl)-4-(4-methylpentanoyl)cyclopent-2-en-1-one, (4S,5S)-3,4-dihydroxy-2-(2-methylbutanoyl)-5-(3-methylbutyl)-4-(4-methylpentanoyl)cyclopent-2-en-1-one, (4S,5R)-3,4-dihydroxy-2-(2-methylbutanoyl)-5-(3-methylbutyl)-4-(4-methylpentanoyl)cyclopent-2-en-1-one, and (4R,5R)-3,4-dihydroxy-2-(2-methylbutanoyl)-5-(3-methylbutyl)-4-(4-methylpentanoyl)cyclopent-2-en-1-one.

In yet other embodiments, the substituted 1,3-cyclopentadione is selected from the group consisting of selected from the group consisting of (4S,5S)-3,4-dihydroxy-4-[(1S)-1-hydroxy-4-methylpentyl]-2-(3-methylbutanoyl)-5-(3-methylbutyl)cyclopent-2-en-1-one, (4S,5S)-3,4-dihydroxy-4-[(1R)-1-hydroxy-4-methylpentyl]-2-(3-methylbutanoyl)-5-(3-methylbutyl)cyclopent-2-en-1-one, (4R,5S)-3,4-dihydroxy-4-[(1R)-1-hydroxy-4-methylpentyl]-2-(3-methylbutanoyl)-5-(3-methylbutyl)cyclopent-2-en-1-one, (4R,5S)-3,4-dihydroxy-4-[(1S)-1-hydroxy-4-methylpentyl]-2-(3-methylbutanoyl)-5-(3-methylbutyl)cyclopent-2-en-1-one, (4R,5R)-3,4-dihydroxy-4-[(1R)-1-hydroxy-4-methylpentyl]-2-(3-methylbutanoyl)-5-(3-methylbutyl)cyclopent-2-en-1-one, (4R,5R)-3,4-dihydroxy-4-[(1S)-1-hydroxy-4-methylpentyl]-2-(3-methylbutanoyl)-5-(3-methylbutyl)cyclopent-2-en-1-one, (4S,5R)-3,4-dihydroxy-4-[(1S)-1-hydroxy-4-methylpentyl]-2-(3-methylbutanoyl)-5-(3-methylbutyl)cyclopent-2-en-1-one, (4S,5R)-3,4-dihydroxy-4-[(1R)-1-hydroxy-4-methylpentyl]-2-(3-methylbutanoyl)-5-(3-methylbutyl)cyclopent-2-en-1-one, (4S,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4S,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4R,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5R)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4S,5R)-3,4-dihydroxy-4-[(1S)-hydroxy-4-methylpent-3-en-1-yl]-2-(2-methylpropanoyl)-5-(3-methylbut-2-en-1-yl)cyclopent-2-en-1-one, (4S,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4S,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4S,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4R,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4S,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, and (4S,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one.

In still other embodiments of this aspect, the solvent for the bi-phasic system is selected from the group comprising water, water containing a buffering agent, water containing a soluble polymer, a pentane, hexane, heptane, octane, methyl acetate, ethyl acetate, propyl acetate, butyl acetate, tert-butyl acetate, methanol, ethanol, propanol, iso propanol, butanol, tert butanol, dimethyl formamide, dimethyl sulfoxide, dichloromethane, chloroform; and acetone, or any combination thereof.

In further embodiments, the bi-phasic solvent system has a partition coefficient between 0.6 and 3.0, while in other embodiments the bi-phasic solvent system has a partition coefficient between 0.7 and 1.5.

The counter current chromatography is performed at a temperature of about 20° C. to about 30° C. in some embodiments of the invention while the counter current chromatography may be performed at ambient temperature in other embodiments.

In some embodiments, the pharmaceutically acceptable excipient is selected from the group consisting of an isotonic and absorption delaying agent, binder, adhesive, lubricant, disintegrant, coloring agent, flavoring agent, sweetening agent, absorbants, detergent, and emulsifying agent, or any combination thereof, while in yet other embodiments, the composition further comprises one or more antioxidants, vitamins, minerals, proteins, fats, and carbohydrates, while in yet other embodiments.

In further embodiments the composition is in a dosage form suitable for administration via a route selected from the group consisting of oral, inhalation, rectal, ophthalmic, nasal, topical, vaginal, and parenteral.

In another embodiment, the congener in the composition is at least eighty percent pure, while in other embodiments the congener is at least ninety-five percent pure.

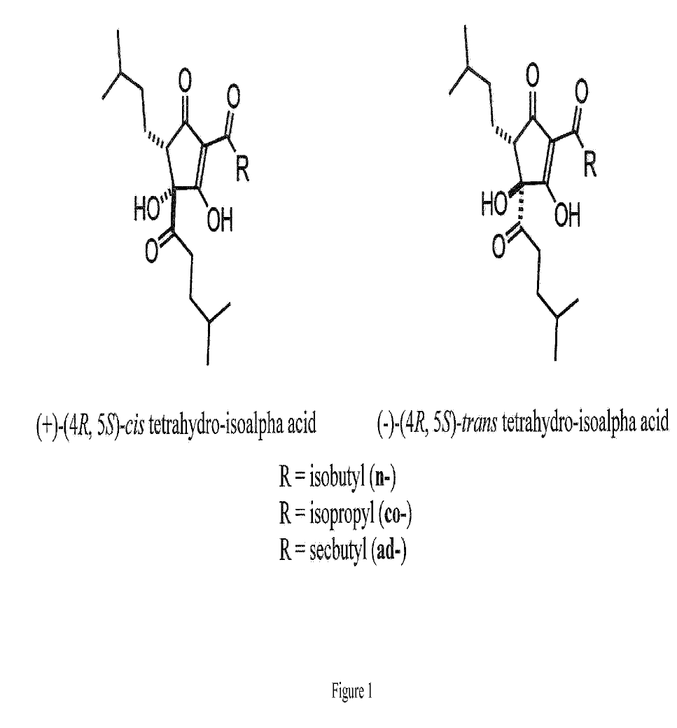

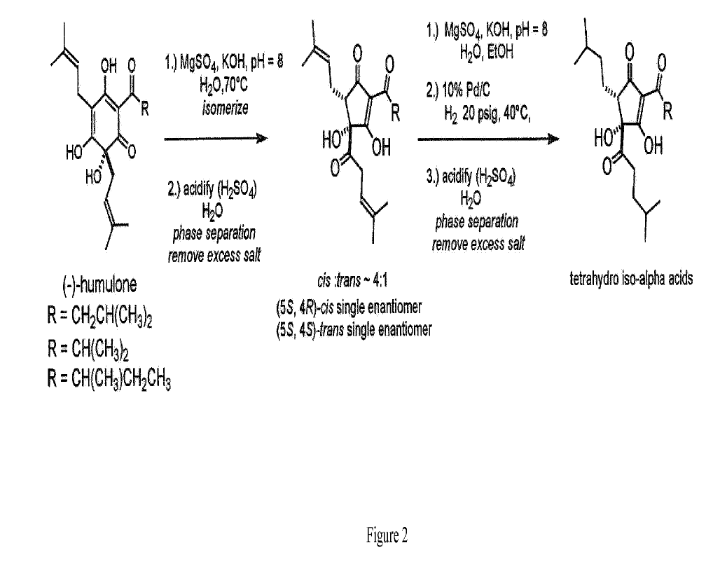

Substituted cyclohexa-2,4-dienones, substituted cyclohexane-1,3,5-triones, substituted cyclopent-2-en-1-ones, and substituted 1,3-cyclopentadiones, are comprised of three structural analogs: n-, co- and ad-. See FIG. 1 for substituted 1,3-cyclopentadiones. Each analog is present as both cis- and trans-diastereomers. The purification of the individual analogs into their respective cis- and trans-diastereomers is a difficult and challenging problem. This invention describes a novel and facile chromatographic purification method of the cis- and trans-diastereomers. The method provides significant quantities, i.e., multiple grams, of pure cis- and trans-diastereomers of each structural analog, which enables the determination of their individual pharmacological and toxicological properties.

The present invention provides a high-speed counter current chromatography (HSCCC) method to enrich or purify diastereomers of substituted cyclohexa-2,4-dienones, substituted cyclohexane-1,3,5-triones, substituted cyclopent-2-en-1-ones, and substituted 1,3-cyclopentadiones, in particular, tetrahydro isoalpha acids. The purification method as applied to tetrahydro isoalpha acids can be similarly applied to other reduced isoalpha acids (i.e., dihydro- and hexahydro-isoalpha acids).

The solvent for the bi-phase system may be, for example, water preferably having a pH between 0 and 14, or preferably a pH between 1 and 13, 3 and 12, or 5 and 10; water containing a buffering agent with a pH between 0 and 14, 1 and 13, 3 and 12, or 5 and 10; water containing a soluble polymer; a pentane; hexane; heptane; octane; methyl acetate; ethyl acetate; propyl acetate; butyl acetate; tert-butyl acetate; methanol; ethanol; propanol; iso propanol; butanol; tert butanol; dimethyl formamide; dimethyl sulfoxide; dichloromethane; chloroform; or acetone, or any combination thereof.

The partition coefficient of the bi-phasic system used for purifying a reduced isoalpha acid is in the range of from about 0.5 to 5, or preferably about 0.6 to 4, 0.7 to 3, 0.8 to 2, 0.85 to 1.5, or 0.9 to 1.2, or most preferably about 0.9 to 1.1.

The counter current chromatography is performed at a temperature of about 20° C. to about 30° C., or preferably about 22° C. to about 28° C., or about 23° C. to about 27° C., but may be performed at ambient temperature.

The structures of the compounds collectively referred to as THIAA are shown in FIG. 1. Due to variation at the acyl side chain, THIAA is primarily composed of three structural analogues, (De Keukeleire, 2000; Verzele, 1986). These three analogues are designated with the following prefixes, n-(isobutyl), co-(isopropyl) and ad-(secbutyl). As shown in FIG. 1, both the cis and the trans diastereomers for each analogue are present in the THIAA mixture. Each diastereomer is produced as a single enantiomer; for this reason a maximum of 6 unique chemical species derived from three analogues, ie, n-, co-, and ad-, may be present in THIAA.

Typically, the process of THIAA manufacture from hops begins with the extraction of hop cones with supercritical carbon dioxide (CO₂) (De Keukeleire et al, 1999; De Keukeleire, 2000). This extraction process begins immediately following the harvesting and collection of the hop cone. The cones are dried, crushed and pressed into pellets. The pellets are loaded into an extractor, and supercritical CO₂ is passed over the pellets at a pressure of 200-300 bar. Extraction is typically carried out at a temperature in the range of 40-60° C. Extracted components flow from the extraction chamber into an evaporation separation tank, wherein the pressure is lowered to 60-80 bar, and the extracted hop components are separated from CO₂.

Following the removal of CO₂ the extract is dissolved in alkaline water, and magnesium sulfate is added. The resulting solution is heated and, under these conditions, the alpha acids undergo a stereospecific isomerization to the isoalpha acids (FIG. 2). Following the conversion of the alpha acids to the isoalpha acids, the solution is acidified with H₂SO₄ and the excess salt is removed from the resulting free acid form of the isoalpha acids by taking advantage of the resulting phase separation and using successive washings with water.

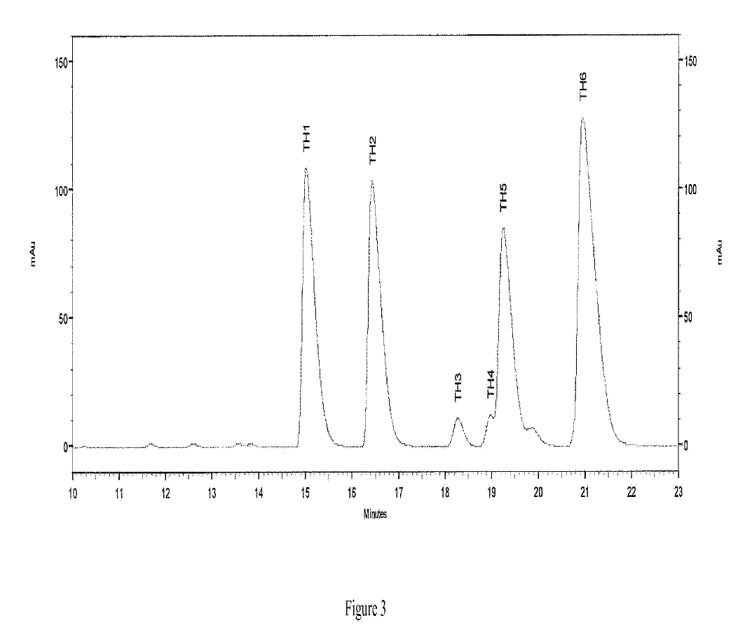

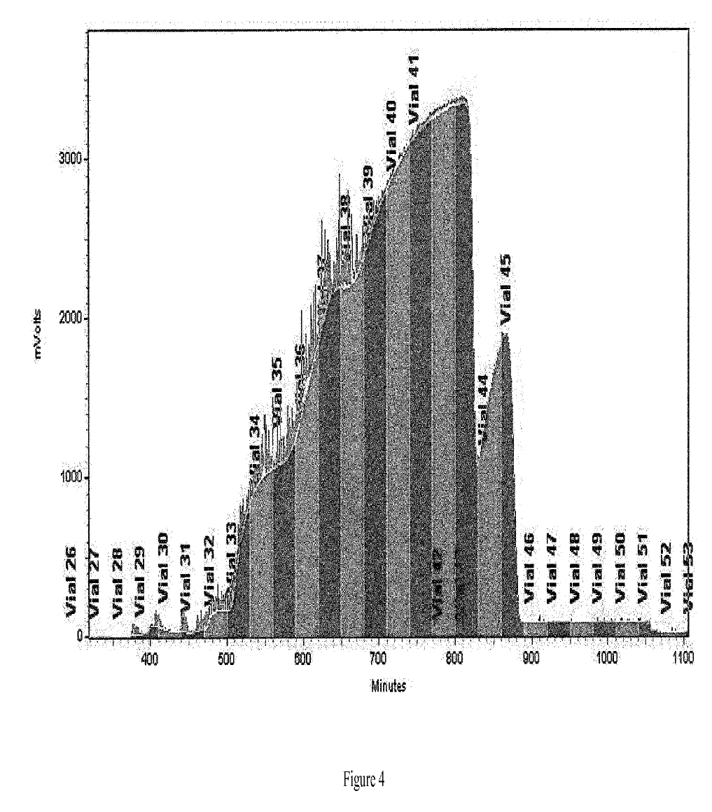

The isoalpha acids (free acid) are dissolved in a 1:9 mixture of alkaline water containing magnesium sulfate (0.5 to 1.0 equivalents; final pH=6.0-8.0) and ethanol. To this solution is added 1% by weight of 10% Pd on carbon catalyst and the solution is placed in a hydrogenation vessel and heated to approximately 40° C. under 20 psig of hydrogen gas. Following several hours under these conditions the iso-alpha acids are reduced to the tetrahydro isoalpha acids (FIG. 3); at this point the 10% Pd on carbon catalyst is removed via filtration. The filtrate is acidified in order to generate the free acid form of the tetrahydro isoalpha acids and the ethanol is removed via distillation. The remaining water insoluble free acid form of the tetrahydro is then phase separated and successively washed in order to wash away the water soluble salts.

The present invention makes use of the separation technology known as, high-speed countercurrent chromatography (HSCCC) to purify or isolate reduced isoalpha acids. Applicants have discovered that the differential partitioning properties of the various tetrahydro reduced isoalpha-acid (THIAA) structural analogs (and their respective isomers) between two chosen immiscible liquid phases can be manipulated to allow separation of the various isomers of THIAA. Using a “shake-flask” method, the partition coefficients for THIAA in a variety of immiscible solvent systems have been determined. It was discovered that a lower, aqueous phase consisting of 0.1M ethanolamine (aq) at pH=7.4 and an upper, organic phase of methyl acetate provides a near optimal partitioning ratio (P) of approximately 1.0. In applying this discovery, an HSCCC instrument (PharmaTech Research, model CCC-1000) can be employed to effect the separation of pure and highly enriched fractions of the various diastereomers present in modified hops extracts containing THIAA. See FIG. 3.

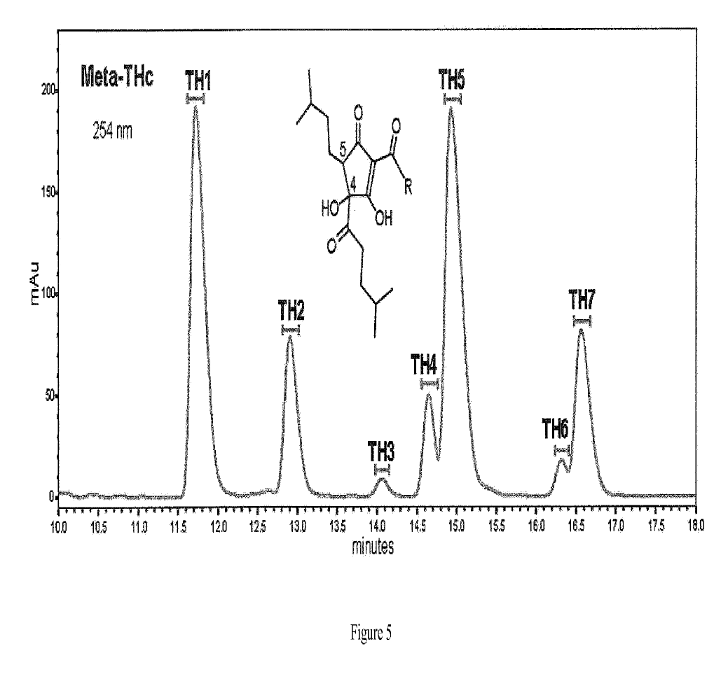

The elution and fractionation of THIAA components can be monitored (FIG. 4) and percent homogeneity of each fraction can be determined; the amount isolated in each fraction and the percent recovery based upon the initial amount of material submitted to HSCCC purification can be assessed. Table 7 below provides results of one such assay:

TABLE 7
Percent homogeneity of each fraction
Purity of CCC fractions based on peak area (HPLC, 254 nm)

| | Vial 32 | Vial 33 | Vial 34 | Vial 35 | Vial 36 | Vial 37 | Vial 38 | Vial 39 | Vial 40 | Vial 41 | Vial 42 | Vial 43 | Vial 44 | Vial 45 |
|---|---|---|---|---|---|---|---|---|---|---|---|---|---|---|
| TH1 | | 79.5 | 82.8 | 77.5 | 57.1 | 38.4 | 11.9 | 0.9 | | | | | | |
| TH2 | 81 | 9.9 | | | | | | | | | | | | |
| TH3 | | | | | | | | | | | | 0.7 | 7.4 | 6.3 |
| TH4 | | | | | | | | | | | | 6.2 | 91.3 | 92.2 |
| TH5 | | | | 3.9 | 28.5 | 52.5 | 84.3 | 97.6 | 98.9 | 99 | 99.1 | 92.4 | | |
| TH6 | | 6.6 | 16.3 | 18.6 | 14.5 | 8.7 | 3.8 | 1.5 | 1.1 | 1 | 0.9 | 0.6 | | |

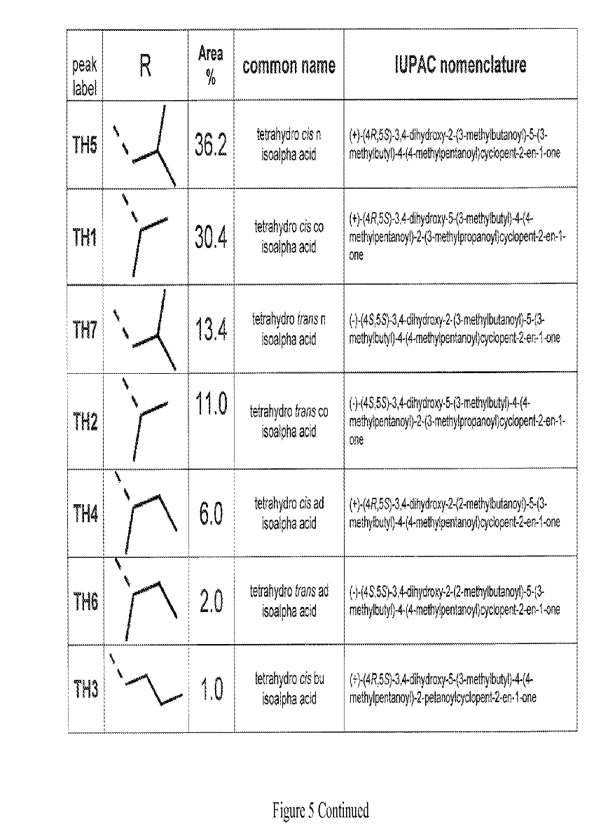

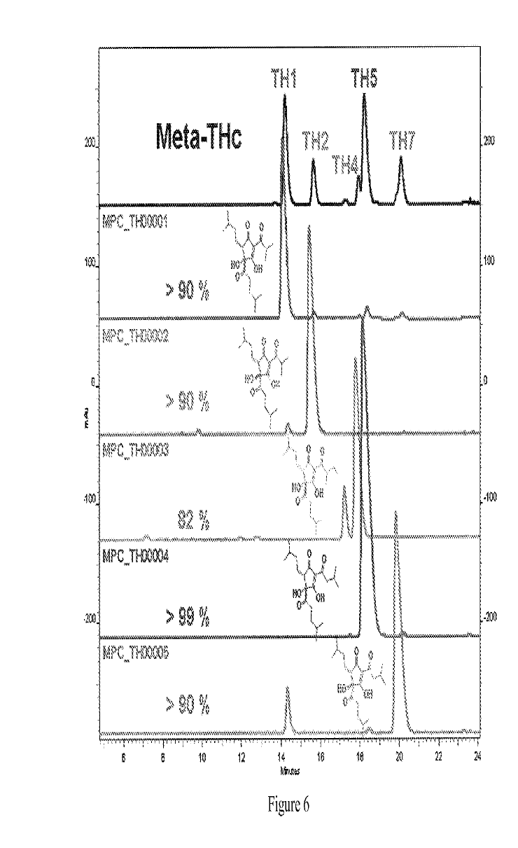

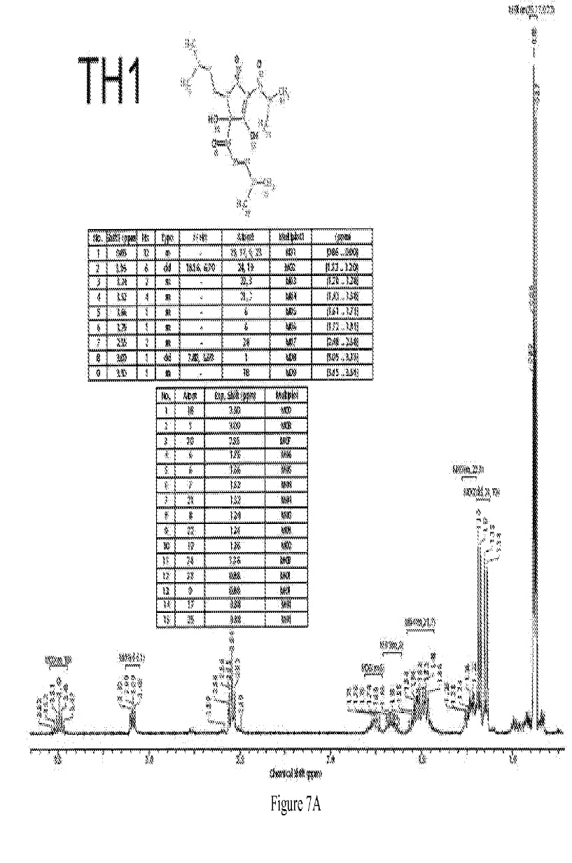

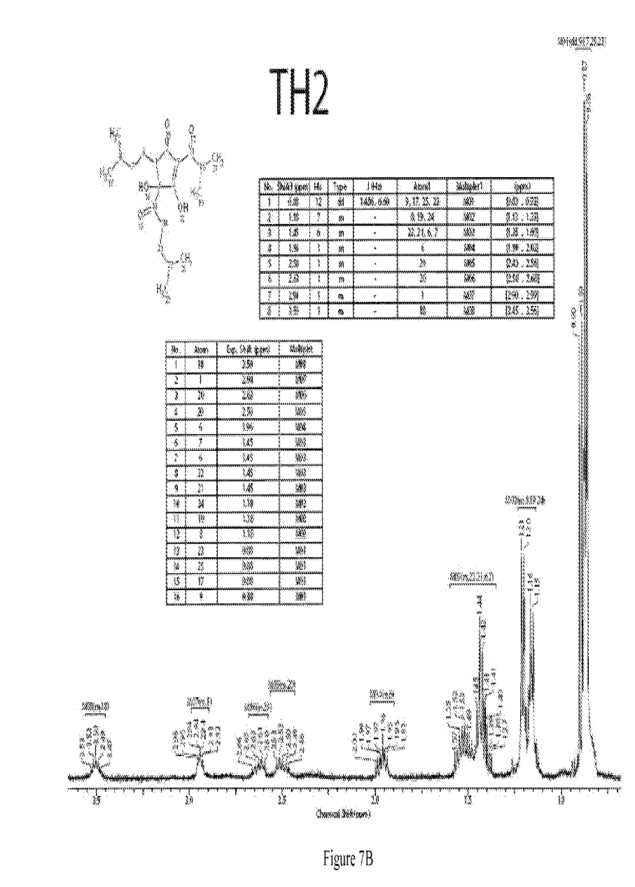

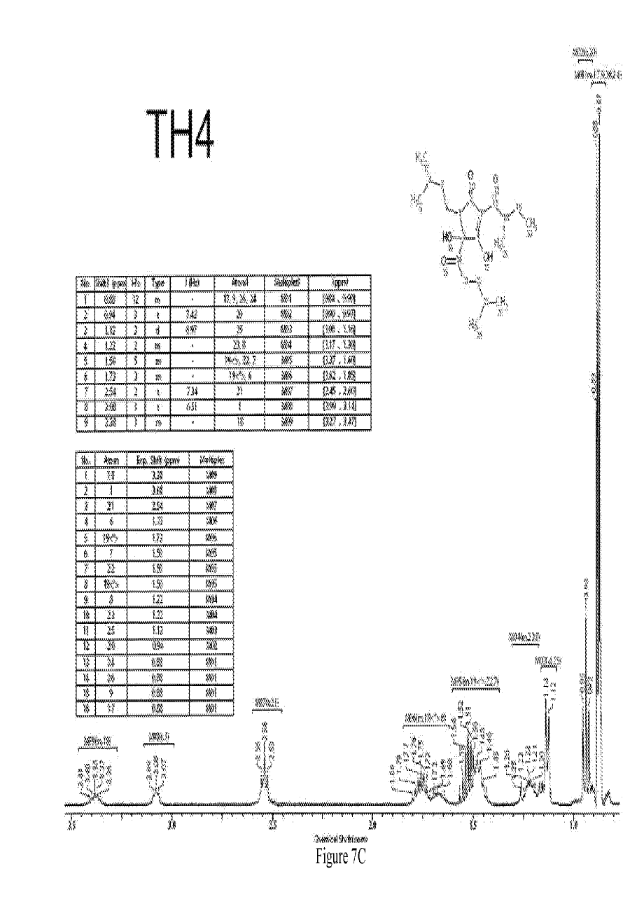

In a repeat study, a more detailed chromatogram of THIAA prior to HSCCC indicated the existence of seven peaks, TH1-7, as shown in FIGS. 5 and 6. The five fractions corresponding to most prominent peaks were obtained and each fraction was analyzed by HPLC, mass spectrometry, HNMR (FIG. 7), and chiral-column HPLC in order to determine the chemical and the optical purity of each fraction. All five fractions were sufficiently pure for biological assay as shown in Table 8.

TABLE 8
Composition of fractions isolated from THIAA

| | ho- | | |
|---|---|---|---|
| | mo- | | |
| | ge- | | |
| | ne- | | |
| | ity | | la- |
| fraction | (%) | Structure | bel |
| MPC_TH0001 | >90 | 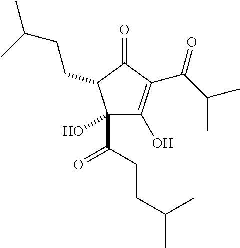 | TH1 |
| MPC_TH0002 | >90 | 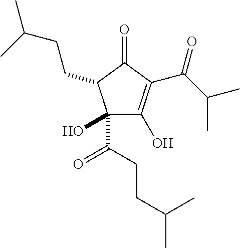 | TH2 |
| MPC_TH0003 | 82 | 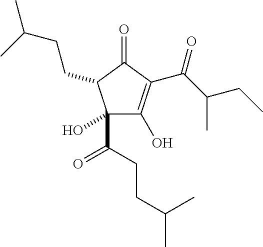 | TH4 |
| MPC_TH0004 | >99 | 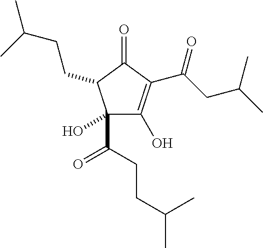 | TH5 |
| MPC_TH0005 | >90 | 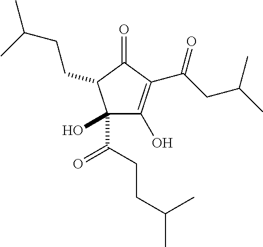 | TH7 |

Each of these fractions can be readily separated using the separation method disclosed herein. Further the disclosed method is applicable to other reduced isoalpha acids, such as Rho isoalpha acids or hexahydro isoalpha acids.

A consistent and accepted nomenclature for each purified compound followed by the IUPAC nomenclature is presented as follows:
TH1: (+)-(4R,5S)-cis-co-tetrahydro isoalpha acid
(+)-(4R,5S)-3,4-dihydroxy-2-isobutyryl-5-(3-methylbutyl)-4-(4-methylpentanoyl)cyclopent-2-en-1-one
TH2: (−)-(4S,5S)-trans-co-tetrahydro isoalpha acid
(−)-(4S,5S)-3,4-dihydroxy-2-isobutyryl-5-(3-methylbutyl)-4-(4-methylpentanoyl)cyclopent-2-en-1-one
TH4: (+)-(4R,5S)-cis-ad-tetrahydro isoalpha acid:
(+)-(4R,5S)-3,4-dihydroxy-2-(2-methylbutanoyl)-5-(3-methylbutyl)-4-(4-methylpentanoyl)cyclopent-2-en-1-one
TH5: (+)-(4R,5S)-cis-n-tetrahydro isoalpha acid:
(+)-(4R,5S)-3,4-dihydroxy-2-(3-methylbutanoyl)-5-(3-methylbutyl)-4-(4-methylpentanoyl)cyclopent-2-en-1-one.
TH7: (−)-(4S,5S)-trans-n-tetrahydro isoalpha acid:
(−)-(4S,5S)-3,4-dihydroxy-2-(3-methylbutanoyl)-5-(3-methylbutyl)-4-(4-methylpentanoyl)cyclopent-2-en-1-one

As used herein, the terms “derivatives” or a matter “derived” refer to a chemical substance related structurally to another substance and theoretically obtainable from it, i.e. a substance that can be made from another substance. Derivatives can include compounds obtained via a chemical reaction.

The term “pharmaceutically acceptable” is used in the sense of being compatible with the other ingredients of the compositions and not deleterious to the recipient thereof.

As used herein, “tetrahydro-isohumulone” shall refer to the cis and trans forms of (+)-(4R,5S)-3,4-dihydroxy-2-(3-methylbutanoyl)-5-(3-methylbutyl)-4-(4-methylpentanoyl)cyclopent-2-en-1-one and (−)-(4S,5S)-3,4-dihydroxy-2-(3-methylbutanoyl)-5-(3-methylbutyl)-4-(4-methylpentanoyl)cyclopent-2-en-1-one respectively.

“Tetrahydro-isocohumulone”, as used herein refers to the cis and trans forms of (+)-(4R,5S)-3,4-dihydroxy-5-(3-methylbutyl)-4-(4-methylpentanoyl)-2-(3-methylpropanoyl)cyclopent-2-en-1-one and (−)-(4S,5S)-3,4-dihydroxy-5-(3-methylbutyl)-4-(4-methylpentanoyl)-2-(3-methylpropanoyl)cyclopent-2-en-1-one respectively.

“Tetrahydro-adhumulone” shall be used herein to refer to the cis and trans forms of (+)-(4R,5S)-3,4-dihydroxy-2-(2-methylbutanoyl)-5-(3-methylbutyl)-4-(4-methylpentanoyl)cyclopent-2-en-1-one and (+)-(4R,5S)-3,4-dihydroxy-5-(3-methylbutyl)-4-(4-methylpentanoyl)-2-petanoylcyclopent-2-en-1-one respectively.

As used herein, “tetrahydro-isoalpha acid” or “THIAA” refers to any mixture of one or more of tetrahydro-adhumulone, tetrahydro-isocohumulone and tetrahydro-isohumulone, including tetrahydro trans n iso-alpha acid, tetrahydro cis n iso-alpha acid, tetrahydro trans n iso-alpha acid, tetrahydro cis co iso-alpha acid, tetrahydro trans co iso-alpha acid, tetrahydro cis co iso-alpha acid, tetrahydro trans co iso-alpha acid, tetrahydro cis ad iso-alpha acid, tetrahydro trans ad iso-alpha acid, tetrahydro cis ad iso-alpha acid, tetrahydro trans ad iso-alpha acid FIG. 1 depicts the chemical structure of the individual members forming tetrahydro-isoalpha acids.

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein.

### EXAMPLES

### Example 1

### GMP Compliant Purification of Tetrahydro Cis n Isoalpha Acid (>99.9%; Hplc Uv/Vis)

A process for the purification of a single phytochemical (tetrahydro cis n isoalpha acid, ((4R,5S)-3,4-dihydroxy-2-(3-methylbutanoyl)-5-(3-methylbutyl)-4-(4-methylpentanoyl)cyclopent-2-en-1-one)) using high speed counter-current chromatography (HSCCC) has been developed as follows:

Description of Equipment & Process

A) HSCCC Equipment: This process was executed on a 100 milligram (analytical) and 1 gram (preparative) scale using a J-type HSCCC instrument (model CCC-1000; Pharma-Tech Research Corp., Baltimore, Md.). The HSCCC instrument contained a self balancing centrifuge rotor equipped with either 3×105 mL coils (analytical) or 3×275 mL coils (preparative). The analytical coils were wrapped with 1.67-mm internal diameter PTFE tubing; the preparative coils were wrapped with 2.65-mm internal diameter PTFE tubing. The revolution radius of the distance between the holder axis and central axis of the centrifuge (R) is 7.5 cm. The β ratio (βᵣ) varied from 0.73 at the head terminal to 0.47 at the tail terminal (βᵣ=r/R, where r is the spool radius and R is the rotor radius). The HSCCC system was equipped with the following Shimadzu (Shimadzu Scientific Instruments, Inc., Columbia, Md.) components: LC-20AT solvent pump with a series-type double plunger, 4 solvent delivery lines and low-pressure mixing; DGU-20A5 solvent degasser; FRC-10A fraction collector & prep collection apparatus; CBM-20A system controller and a SPD-10AV vp UV detector. The sample injection was performed using a Rheodyne® model 3725i manual injection valve (Oak Harbor, Wash.) equipped with either a 10 mL or 100 mL sample loop. These components were controlled using a computer workstation (HP Compaq dc5100, MS Windows XP v. 2002) running Shimadzu EZ Start 7.4.

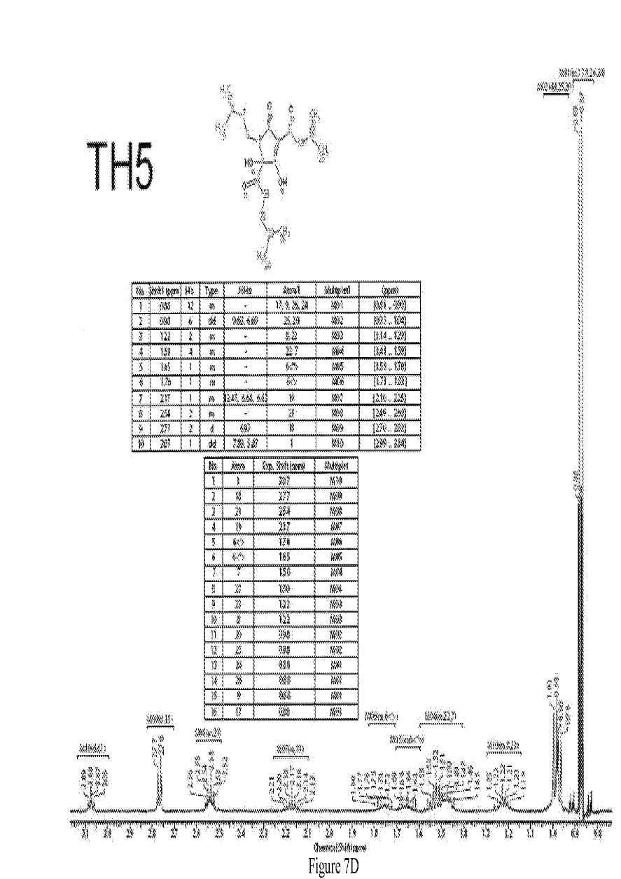

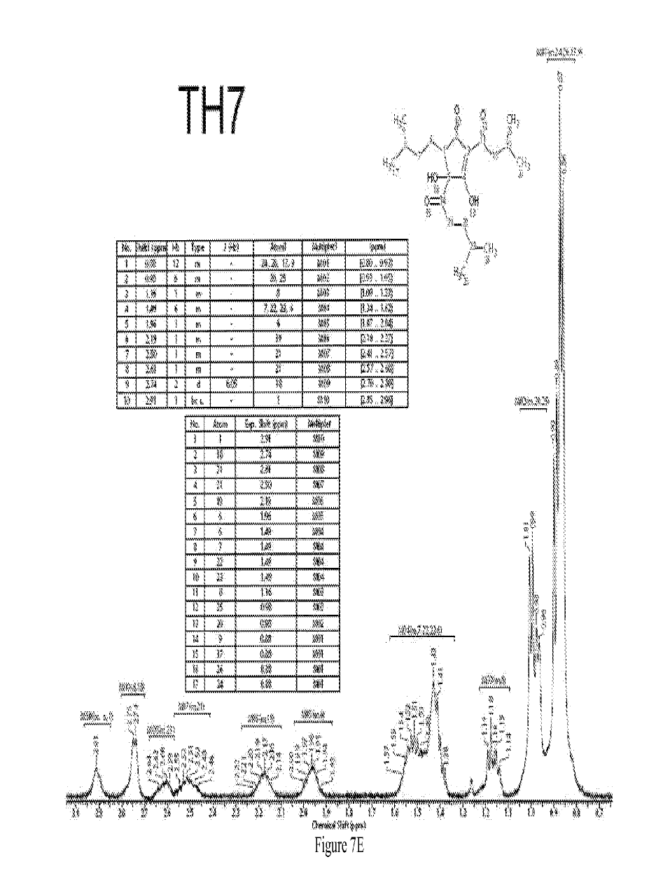

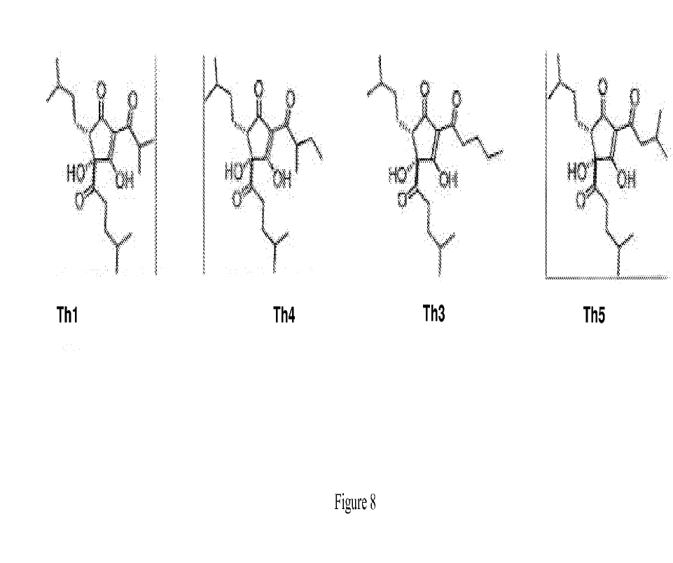

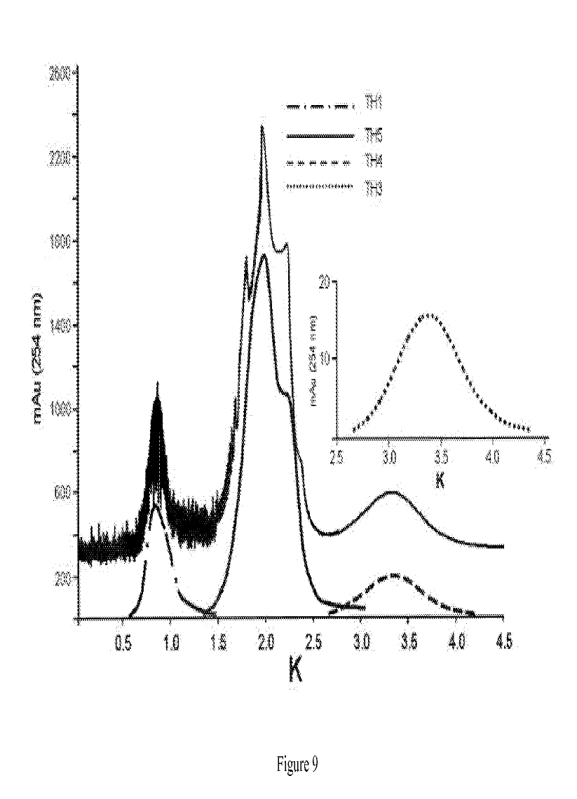

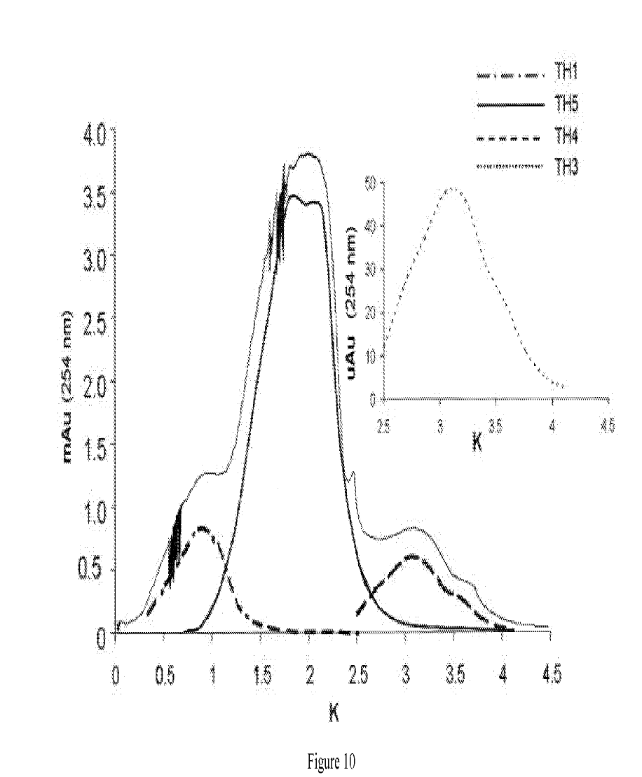

B) Raw Material: The material submitted for the purification of tetrahydro cis n isoalpha acid is provided by MetaProteomics and consists of a single diastereomeric (cis) mixture of congeners of the so-called tetrahydro isoalpha acids (THIAA). The raw material consists of approximately 80%-90% (w/w) tetrahydro isoalpha acids (THIAA), (4R,5S)-3,4-dihydroxy-2-(3-methylbutanoyl)-5-(3-methylbutyl)-4-(4-methylpentanoyl)cyclopent-2-en-1-one (tetrahydro cis n isoalpha acid) is present in the raw material in the range of 50-60% (w/w); the remaining material consists of a variety of low molecular weight carboxylic acids and hydrocarbons. The predominant cis tetrahydro isoalpha acids are shown in FIG. 8.

C) HSCCC method: The separation was performed in descending mode where the stationary phase was the lighter (upper) phase and the mobile phase was the denser (lower) phase. The elution of mobile phase proceeds in a “head-to-tail” direction. The stationary phase consists of HPLC grade hexanes, the mobile phase consists of a 250 mM NH₄PO₄ aqueous buffer at pH=6.3; both phases are thoroughly mixed and equilibrated in an approximate 1:1 ratio prior to use. The HSCCC is initially charged with stationary phase at 8 mL/min. Following the complete filling of the coils with stationary phase, the coils were rotated at 700 rpm and charged with mobile phase at either 2 mL/min (analytical coils) or 4 mL/min (preparative coils). The eluent was collected so that the volume of stationary phase that elutes prior to the elution of the mobile phase could be measured. This volume of stationary phase was used to check for a satisfactory retention of stationary phase in the coils. Following the elution of mobile phase, the raw material was dissolved in a bi-phasic mixture (1:1 v/v) of upper and lower phases for a total concentration of 10 mg/mL. This bi-phasic mixture was then loaded into either a 10 mL (analytical) or 100 mL (preparative) sample loop and injected.

The results for two separate separations on an analytical (100 mg) and preparative (1000 mg) scale are shown in FIGS. 9 & 10 respectively. FIG. 11 summarizes the amount of purified material recovered and the percent homogeneity of each component.

Following the completion of the run, each of the individual fractions collected were analyzed via HPLC and the enriched fractions were pooled into a separatory funnel. The mobile phase was then acidified (H₂SO₄ cone) to pH=2.0 and extracted with hexanes 3×. The hexanes were collected and removed in vacuo to yield pure component.

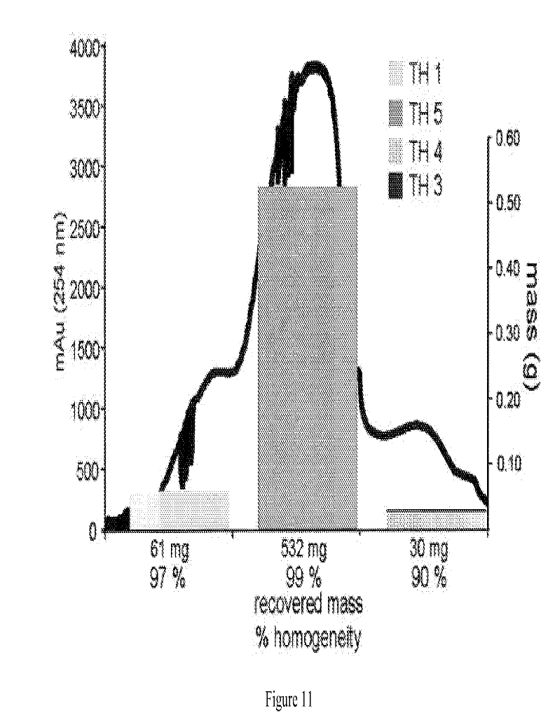

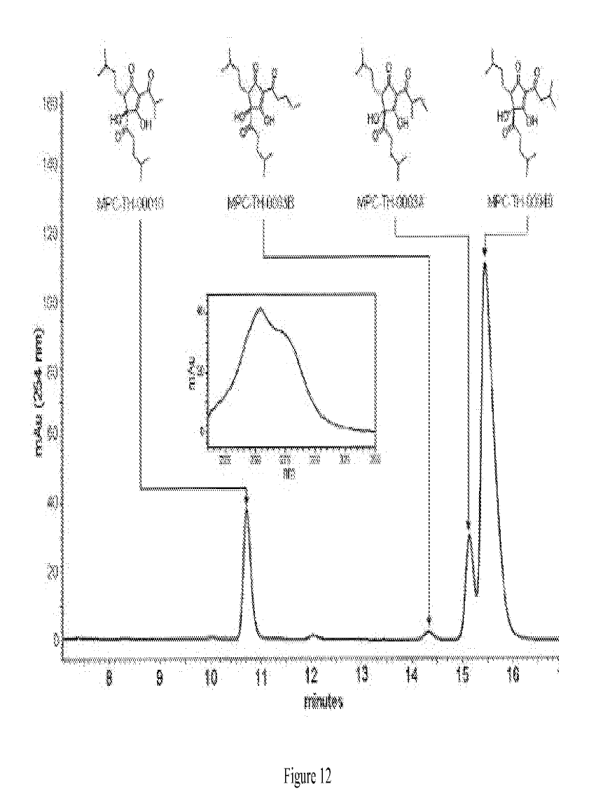

D) HPLC equipment and method: The analysis of the raw material and the homogeneity of the purified components was performed using HPLC. A representative HPLC chromatogram of the raw material is shown in FIG. 12.

HPLC analyses were performed using a Shimadzu HPLC system (Shimadzu Scientific Instruments, Inc., Columbia, Md.). The HPLC system consists of a DGU 14A solvent degasser, LC-10AD solvent pumps (3), SPD-M10ADVP photodiode array detector monitoring at 254 nm, SIL-10ADVP auto injector, SCL10AVP system controller and a CTO-10AVP column oven operating. This system is controlled using Class VP 7.3 sp1 software. A 250×4.6 mm Gemini NX C18, 3u, 110A (Phenomenex, Torrance, Calif.) column with matching guard column was used for the HPLC analysis. The separation method employs two mobile phases, A and B; solvent A was a 20 mM NH₄Ac aqueous buffer at pH 9.5; solvent B is a binary mixture of acetonitrile and methanol in a 6:4 (v/v) ratio. The method was performed with a flow rate of 1.6 mL/min at 40° C. using an isocratic elution (44% B) followed by a column wash (95% B) and column re-equilibration. The entire method was completed in 30 min.

### Example 2

### Scale-Up Study

The objective of this study was to scale up the purification process for the purification of a single phytochemical (4R,5S)-3,4-dihydroxy-2-(3-methylbutanoyl)-5-(3-methylbutyl)-4-(4-methylpentanoyl)cyclopent-2-en-1-one (tetrahydro cis n isoalpha acid, see FIG. 8) using high speed counter-current chromatography (HSCCC).

The material submitted for the purification is tetrahydro cis n isoalpha acid, and consists of a single diastereomeric (cis) mixture of congeners of the so-called tetrahydro isoalpha acids (THIAA). The raw material consists of approximately 80%-90% (w/w) tetrahydro isoalpha acids, Th5 is present in the raw material in the range of 50-60% (w/w); the remaining material consists of a variety of low molecular weight carboxylic acids and hydrocarbons. The predominant cis tetrahydro isoalpha acids are shown in FIG. 8. These compounds are acidic in nature and likely to have high polarity under neutral conditions.

Experimental:

The experimental conditions run on the Spectrum HPCCC instrument were: Solvent system: Stationary phase—Heptane; Mobile phase: Water+ammonium phosphate buffer (pH 6.3); Loading—100 mg in 10 ml; Column volume 136 ml-6 ml/min flow rate.

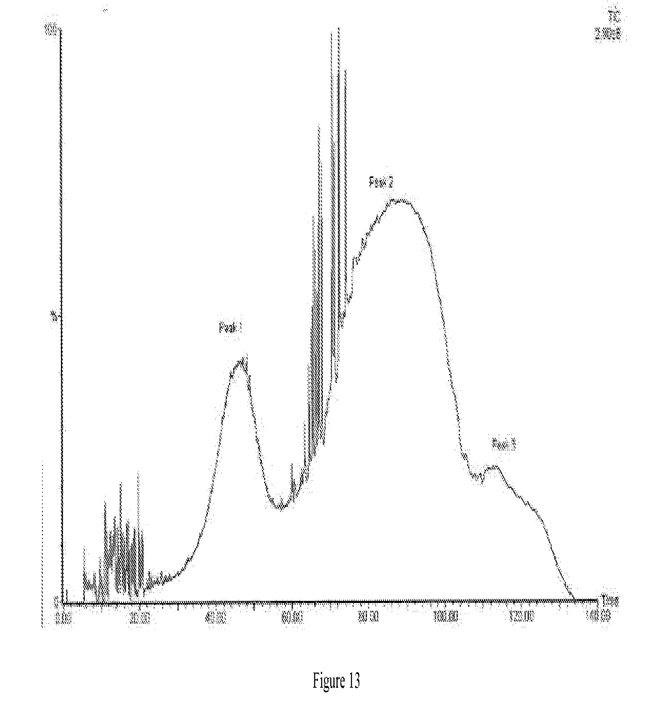

The resulting HPCCC chromatogram is shown below in FIG. 13. There was some SP stripping despite the initial high SP retention, and this is most likely due to the high sensitivity of this solvent system to changes in pH that occur as the sample elutes. The results can be summarized as follows: Separation of 3 peaks achieved; 130 min run time; Total solvent usage—780 ml; Very high SP retention observed ˜95%; Slightly higher retention of compounds than attained by HSCCC machine; Successful separation indicated by HPLC analysis.

Fractions from the middle of the peaks (as shown in FIG. 13) were analyzed. Peak 1: 38-44 min; Peak 2: 80-92 min; Peak 3: 106-122 min. FIG. 14 shows an overlay of the 3 fractions analyzed and the crude material. FIG. 15 is a stack of the 4 chromatograms. The data indicates that separation has been achieved (Peaks at ˜16 min should be ignored as they are due to column contamination).

The result indicated that the purification method could be scaled up with significant improvement in processing capacity. The result showed a throughput improvement by a factor of nine.

### Example 3

### Shake-Flask Experiments and Analyses of Liquid Distribution Ratios

Several shake-flask experiments were conducted to test several solvent systems and determine the liquid distribution ratio (Kd) and partition coefficient values for optimal separations. Partition coefficient values derived from shake-flask experiments are a reliable predictor of countercurrent chromatographic behavior. Kd is the concentration of the analyte in the upper phase divided by the concentration of the analyte in the lower phase and, in the alternative, may be listed as K_{u/l}.

General Procedure and Parameters for Shake Flask Experiments: Addition of 1 mL of the stock buffer at a known pH. Addition of the other necessary solvents to make up the system. Addition of the analyte mixture dissolved in hexane at 400 mg/mL. Final analyte concentration in the shake flask is 0.1 mg/mL. The flask was capped and mixed (by a vortex mixer) for 10 seconds. An aliquot was taken from the upper phase for HPLC analysis, and additionally a separate aliquot was taken from the lower phase for HPLC analysis from each flask. Each component was analyzed by HPLC and integrated using Shimadzu Class VP Software version 7.3 SP1.

Results:

H:E:M:Wat (Hexane:Ethyl acetate:Methanol:Water) Stock of 7:3:5:4:1—THIAA Free Acid at 0.1 mg/mL; 10 uL Injection

| | Stock | TH | TH | TH | TH | TH | TH |
|---|---|---|---|---|---|---|---|
| Buffer | pH | 1 | 2 | 3 | 4 | 5 | 8 |
| HEMWat | | | | | | | |
| # | −2 | 1/Kd | | | | | |
| NH4Ac | 5.3 | 12.2 | 19.3 | 6.1 | 7.1 | 8.1 | 12.3 |
| NH4Ac | 4.8 | 3.4 | 6.0 | 1.7 | 1.8 | 2.5 | 3.5 |
| NH4Ac | 4.3 | 1.6 | 2.8 | 0.8 | 0.9 | 1.2 | 1.7 |
| HEMWat | | | | | | | |
| # | −2 | Kd | | | | | |
| NH4Ac | 5.3 | 0.08 | 0.05 | 0.16 | 0.14 | 0.12 | 0.08 |
| NH4Ac | 4.8 | 0.29 | 0.17 | 0.58 | 0.56 | 0.41 | 0.28 |
| NH4Ac | 4.3 | 0.61 | 0.35 | 1.22 | 1.15 | 0.81 | 0.61 |

Hexane:Water:Buffer of 10:9:1—Ammonium Counterions; Cis THIAA 0.1 mg/mL

| NH4PO4 | Kd | | | |
|---|---|---|---|---|
| pH | TH 1 | TH 3 | TH 4 | TH 5 |
| 8.2 | 0.0 | 0.0 | 0.1 | 0.0 |
| 7.2 | 0.1 | 0.1 | 0.3 | 0.2 |
| 6.1 | 0.9 | 58.3 | 3.6 | 2.0 |
| NH4 Citrate | Kd | | | |
| pH | TH 1 | TH 3 | TH 4 | TH 5 |
| 6.4 | 1.1 | 2.6 | 3.9 | 2.2 |
| 5.4 | 5.4 | 12.6 | 8.5 | 11.6 |
| 4.6 | 35.0 | 91.6 | 79.7 | 62.6 |

Hexane:Water:Buffer of 10:9:1—Potassium Counterions; Cis THIAA 0.1 mg/mL

| KPO4 | Kd | | | |
|---|---|---|---|---|
| pH | TH 1 | TH 3 | TH 4 | TH 5 |
| 8.2 | 0.0 | 0.0 | 0.1 | 0.0 |
| 7.2 | 0.1 | 0.5 | 0.5 | 0.2 |
| 6.1 | 1.6 | 35.5 | 5.5 | 3.3 |
| Kcitrate | Kd | | | |
| pH | TH 1 | TH 3 | TH 4 | TH 5 |
| 6.4 | 0.8 | 12.8 | 3.1 | 1.8 |
| 5.2 | 8.7 | 68.5 | 21.2 | 17.7 |
| 4.4 | 45.7 | 84.5 | 78.2 | 67.1 |

Hexane:Water:Buffer of 10:9:1—Sodium Counterions; Cis THIAA 0.1 mg/mL

| Na PO4 | Kd | | | |
|---|---|---|---|---|
| pH | TH 1 | TH 3 | TH 4 | TH 5 |
| 8.0 | 0.0 | 0.2 | 0.2 | 0.1 |
| 7.4 | 0.1 | 0.5 | 0.5 | 0.3 |
| 6.2 | 0.8 | 2.9 | 2.9 | 1.7 |
| NaCitrate | Kd | | | |
| pH | TH 1 | TH 3 | TH 4 | TH 5 |
| 6.4 | 2.0 | 8.8 | 6.9 | 4.0 |
| 5.3 | 23.0 | 61.7 | 75.0 | 42.2 |
| 4.4 | 49.2 | 83.7 | 105.3 | 69.8 |

Hexane:Water:Buffer of 10:9:1—Isocratic pH 6.4; Cis THIAA 0.1 mg/mL

| | | Avg Kd | | | | |
|---|---|---|---|---|---|---|
| Cation | Anion | TH 1 | TH 3 | TH 4 | TH 5 | TH 8 |
| Na | Citrate | 2.0 | 6.7 | 5.2 | 4.3 | 3.0 |
| NH4 | Citrate | 1.3 | 3.6 | 4.9 | 2.5 | 1.9 |
| K | Citrate | 1.0 | 2.7 | 4.0 | 2.2 | 1.5 |
| Na | PO4 | 0.9 | 2.4 | 3.3 | 1.5 | 1.2 |
| NH4 | PO4 | 0.7 | 2.0 | 2.8 | 1.3 | 1.0 |
| K | PO4 | 1.0 | 2.9 | 3.3 | 2.0 | 1.5 |

Hexane:Water:Buffer of 10:9:1

| | | | | | | |
|---|---|---|---|---|---|---|
| Buffer | Cation | pH | TH 1 | TH 3 | TH 4 | TH 5 |

THIAA Kd Values Aug. 22, 2008 Page 121

| | | | | | | |
|---|---|---|---|---|---|---|
| Phosphate | NH4 | 6.6 | 0.3 | 0.9 | 1.1 | 0.6 |
| Phosphate | NH4 | 6.7 | 0.3 | 1.0 | 1.1 | 0.6 |

THIAA Aug. 25, 2008 Kd Values Page 122

| | | | | | | |
|---|---|---|---|---|---|---|
| Citrate | NH4 | 6.0 | 0.8 | 4.1 | | 21.2 |
| Citrate | K | 6.1 | 0.1 | 0.3 | 0.5 | 0.2 |
| Phosphate | K | 6.3 | 0.2 | 1.2 | 1.3 | 0.4 |
| Phosphate | NH4 | 6.3 | 0.6 | 2.6 | 2.5 | 1.2 |
| Citrate | K | 6.4 | 0.5 | 2.5 | 2.4 | 1.1 |
| Citrate | NH4 | 6.4 | 0.1 | 0.6 | 0.7 | 0.2 |
| Phosphate | NH4 | 6.6 | 0.1 | 0.4 | 0.5 | 0.2 |
| Phosphate | K | 6.7 | 0.3 | 1.3 | 1.3 | 0.7 |

Hexane:Water:Buffer of 10:9:1

THIAA Kd Values Aug. 21, 2008 Pages: 118-119

| Buffer | Cation | pH | TH 1 | TH 3 | TH 4 | TH 5 |
|---|---|---|---|---|---|---|
| Citrate | Na | 6.1 | 1.9 | 5.3 | 6.4 | 3.9 |
| Citrate | Na | 6.3 | 1.4 | 3.8 | 4.4 | 2.9 |
| Phosphate | Na | 6.3 | 0.6 | 3.7 | 2.6 | 1.3 |
| Phosphate | Na | 6.6 | 0.5 | 0.8 | 1.8 | 1.0 |
| Phosphate | Na | 6.9 | 0.2 | 0.8 | 1.0 | 0.5 |

Hexane:Water:Buffer of 10:9:1

THIAA Aug. 5, 2008 K Value

| pH | TH 1 | TH 2 | TH 3 | TH 4 | TH 5 | TH 8 |
|---|---|---|---|---|---|---|
| 4.9 | 16.5 | 10.8 | 60.3 | 55.6 | 32.8 | 25.6 |
| 5.5 | 4.2 | 2.5 | 17.8 | 16.1 | 9.5 | 6.8 |
| 5.6 | 3.0 | 1.8 | 11.4 | 11.0 | 6.6 | 4.8 |
| 6.0 | 1.5 | 0.9 | 5.5 | 5.5 | 3.3 | 2.5 |

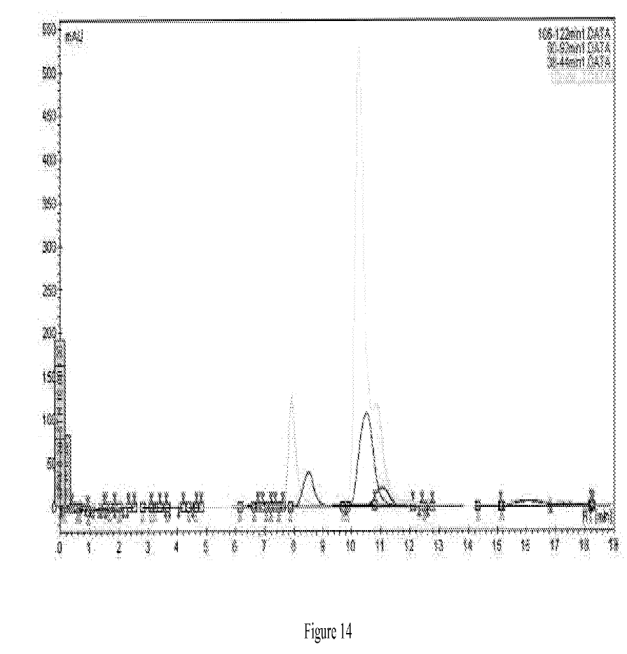

FIG. 16 further shows the result of an HSCCC purification process using specific parameters depicted.

### Example 4

### Purification of Individual Cis Tetrahydro Iso-Alpha Acid Congeners from a Mixture of cis THIAA Congeners via Hydrodynamic/J-Type HSCCC using 325 mL Coil Volume, an “Ascending” Elution Method and a pH=6.79

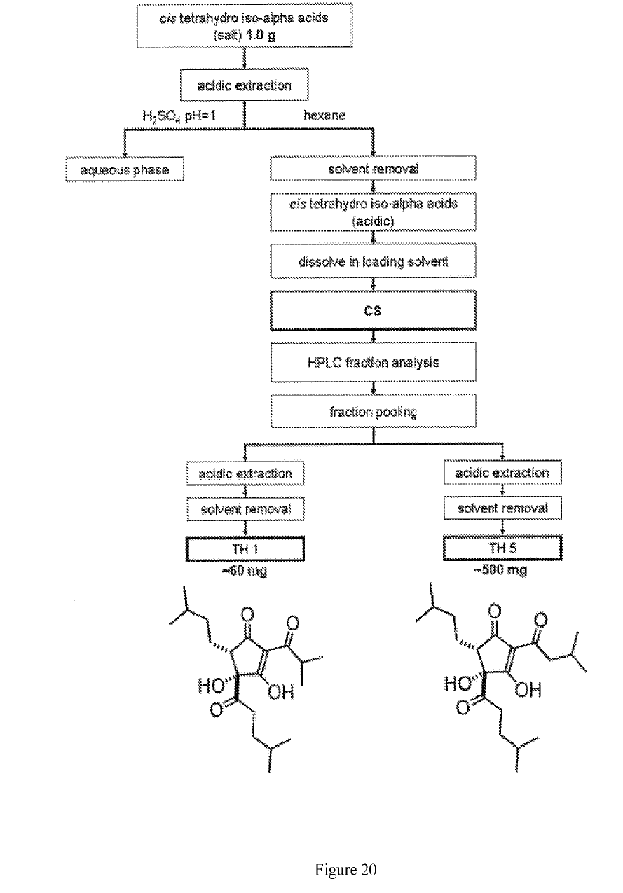

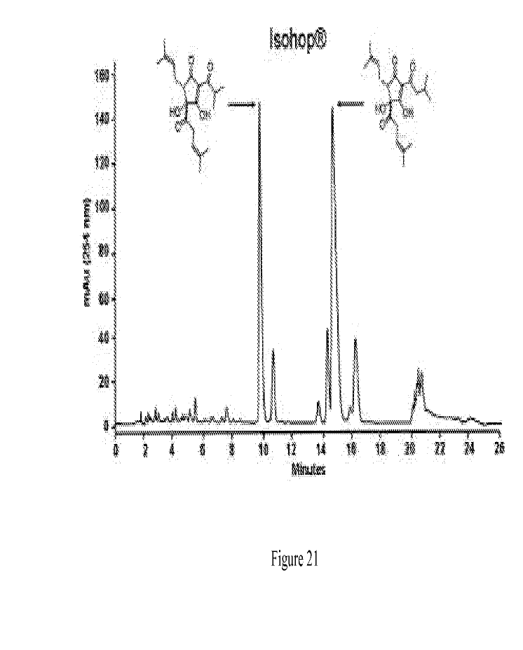

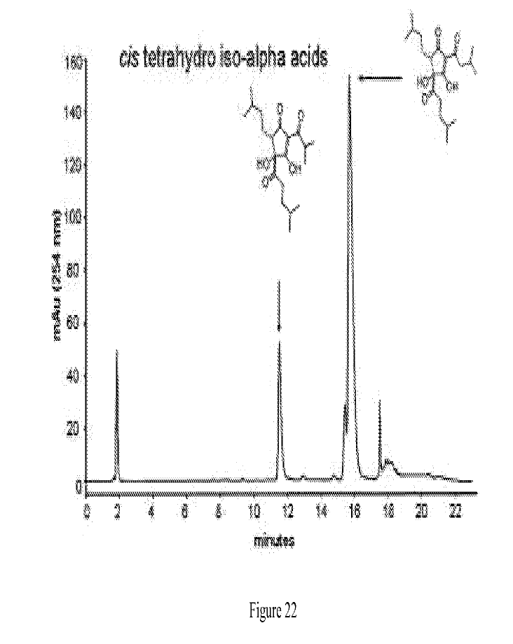

The purification of individual cis tetrahydro iso-alpha acids (cis-TIHAA) congeners, beginning with a mixture of cis THIAA congeners, was performed according to the flow chart depicted in FIG. 20. The starting material, a mixture of cis tetrahydro iso-alpha acids (cis-TIHAA) congeners, was obtained form Hop Steiner, Yakima, WA. An HPLC analysis of this mixture is shown in FIG. 22. The solvent system for the HSCCC purification was made by combining 1000 mL of hexane, 900 mL of water, 65 mL of concentrated ammonium hydroxide ([14.5 M] 71% H₂O), and 35 mL of concentrated phosphoric acid ([14.8 M] 15% H₂O) in a separatory funnel followed by vigorous shaking. Following the settling of two immiscible phases in the separatory funnel, the pH was measured in the aqueous phase using a calibrated pH meter and determined to be 6.79. The organic “upper” phase and the aqueous “lower” phase were separately collected from the separatory funnel. The lower phase was used as the stationary phase, and henceforth used to initially charge the 320 mL coils of a hydrodynamic/J-type HSCCC instrument (PharmaTech Research, CCC-1000). Following the complete filling of the 325 mL coils, the HSCCC was spun at 680 RPM and the “upper” phase was pumped at a rate of 4 mL/min in a ‘tail-to-head elution’ also referred to as an “ascending” or “normal phase” elution mode. When the upper phase eluted from head of the column equilibration was complete, and the equilibration i.e., pumping the “upper” phase at a rate of 4 mL/min was continued.

The mixture of cis THIAA (free acidic) congeners was dissolved in the “upper” phase to make a stock solution at a concentration of 200 mg/mL. 2.5 mL of this stock solution was drawn into a 10 mL syringe followed by an additional 2.5 mL of “upper” phase, thus bringing the total volume within the syringe to 5.0 mL and the total concentration of the mixture of cis THIAA congeners in the syringe to 100 mg/mL. This sample was then injected into a 3.8 mL sample loop. This injection volume overfills the sample loop, thus ensuring a reproducible sample injection volume. In this example, 380 mg of the mixture of cis THIAA congeners was loaded onto the HSCCC. The UV detector began to monitor the eluent coming out of the HSCCC coils immediately following the sample-loop injection. The flow rate of the “upper” mobile phase continued at 4 mL/min and fractions were collected in 28 mL increments following 40 minutes after the sample-loop injection. After 150 minutes, the “upper” phase was no longer used as the mobile phase; instead additional “lower” stationary phase was pumped into the coils at a flow rate of 4 ml/min. The replacement of the “upper” phase with the “lower” phase marks the beginning of the elution-extrusion method; the total run time was 250 minutes. Following 250 minutes of elution from the sample-loop injection, all of the collected fractions were analyzed using HPLC in order to determine which fractions contained homogenous solutions of purified cis THIAA congeners. Upon completing the HPLC analysis of each fraction, a CS trace was reconstructed by plotting the peak area of each of the purified cis THIAA congeners versus time (minutes) or several other parameters e.g., volume (mL) or “K”, as shown in FIG. 23. Based upon the homogeneity of the collected fractions, they were pooled accordingly and extracted into hexanes via an acidic aqueous extraction as described in FIG. 20. This final acidic extraction is conducted in order to remove any residual water form the fractions. The hexanes were removed in vacuo to yield highly pure cis THIAA congeners. This procedure rendered 30 mg of TH 1, and 228 mg of TH 5, respectively, in >85% purity as determined by HPLC.

### Example 5

### Purification of Individual Cis Tetrahydro Iso-Alpha Acid Congeners from a Mixture of cis THIAA Congeners Via Hydrodynamic/J-Type HSCCC Using an 825 ml Coil Volume, a “Descending” Elution Method and a pH=6.34

This example followed procedures as described in Example 1. The purification of individual cis-TIHAA congeners, from a mixture of cis-TIHAA congeners in this example follows the flow chart depicted in FIG. 20. Several differences between this example and Example 1 are noted. In this example the amount of the mixture of cis THIAA congeners submitted for purification is greater, the coil volume of the HSCCC is greater (820 mL versus 325 mL), this example employs a “descending” method of elution and the pH of the aqueous phase in this example is lower than the pH used in Example 1 (6.34 versus 6.79). Lastly, the manner in which the sample was loaded onto the HSCCC coils in this example is significantly different compared to Example 1.

The solvent system for this particular example was made by equilibrating 1000 mL of hexane, 3800 mL of water, 66 mL of ammonium hydroxide ([14.5 M] 71% H₂O), and 42.3 mL of phosphoric acid ([14.8 M] 15% H₂O) in a separatory funnel followed by vigorous shaking. Following the settling of two immiscible phases in the separatory funnel, the pH was measured in the aqueous phase using a calibrated pH meter and determined to be 6.34. The organic “upper” phase was separately collected from the aqueous “lower” phase. The upper phase was used as the stationary phase, and henceforth used to initially charge the 820 mL coils. Upon complete coil filling, the HSCCC was spun at 700 RPM, and the “upper” phase was pumped at 4 mL/min in a ‘head-to-tail’ elution. Following the complete filling of the 820 mL coils, the HSCCC was spun at 680 RPM and the “lower” phase was pumped at a flow rate of 4 mL/min in a ‘head-to-tail’ orientation, also referred to as “descending” or “reverse phase” elution. When the “lower” phase eluted from head of the coils, the equilibration was complete, and the “lower” phase continued to flow through the coils at a flow rate of 4 mL/min.

1021 mg of a mixture of cis tetrahydro iso-alpha acid (THIAA) congeners (free acid) was dissolved in 50 mL of the “upper” phase, 40 mL of the “lower phase”, and 10 mL of absolute ethanol. The sample was then filtered through a 1 um syringe filter and injected into an empty 100 mL sample loop. At this time the HSCCC was fully equilibrated and the entire sample loop volume was injected onto the HSCCC coils. The UV detector monitored the eluent coming out of the HSCCC coils immediately following the sample-loop injection. Fractions were collected in 93 mL volumes following the first 50 minutes after the sample-loop injection; the total run time was 750 minutes.

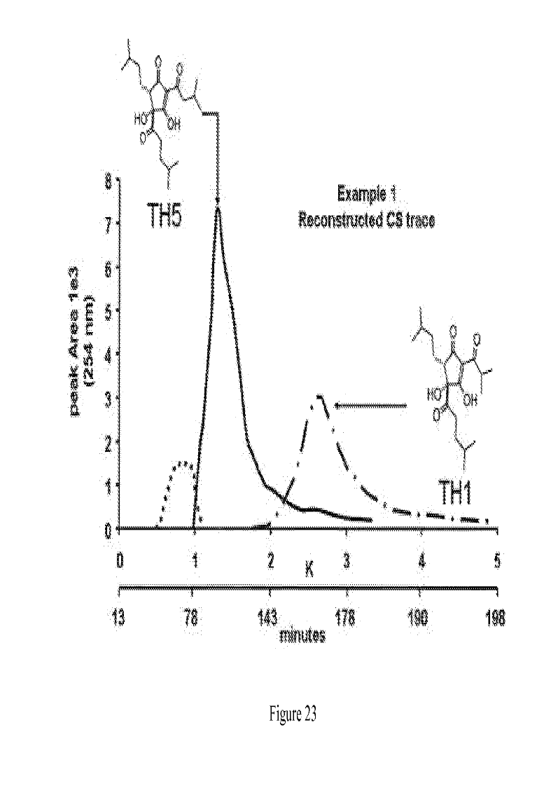

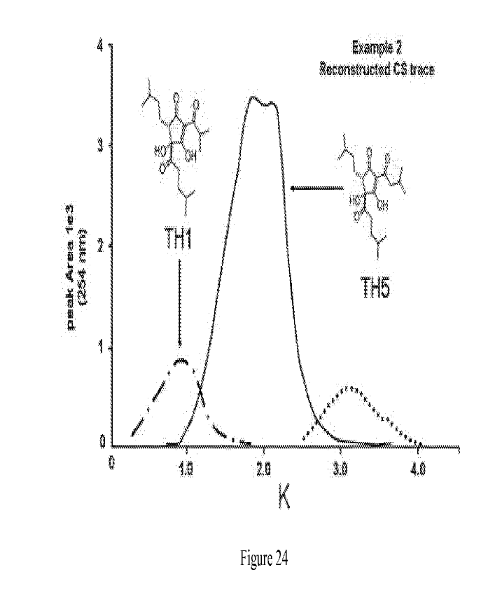

All of the collected fractions were analyzed using HPLC in order to determine which fractions contained homogenous solutions of purified cis THIAA congeners. Upon completing the HPLC analysis of each fraction, a CS trace was reconstructed by plotting the peak area of each of the purified cis THIAA congeners versus time (minutes) or several other parameters e.g., volume (mL) or “K”, as shown in FIG. 24. Based upon the homogeneity of the collected fractions, they were pooled accordingly and extracted into hexanes via an acidic aqueous extraction as described in FIG. 20. This final acidic extraction is conducted in order to remove the aqueous “lower” phase (used as the mobile phase in descending mode) form the fractions. The hexanes were removed in vacuo to yield highly pure cis THIAA congeners. This procedure rendered 60.7 mg of TH 1, and 531 mg of TH 5, respectively, in >95% purity as determined by HPLC.

### Example 6

### Purification of Individual Cis Iso-Alpha Acid Congeners from a Mixture of Cis Iso-Alpha Acid Congeners Via Hydrodynamic/J-Type HSCCC Using an 825 ml Coil Volume, an “Ascending” Elution Method and a pH=4.92

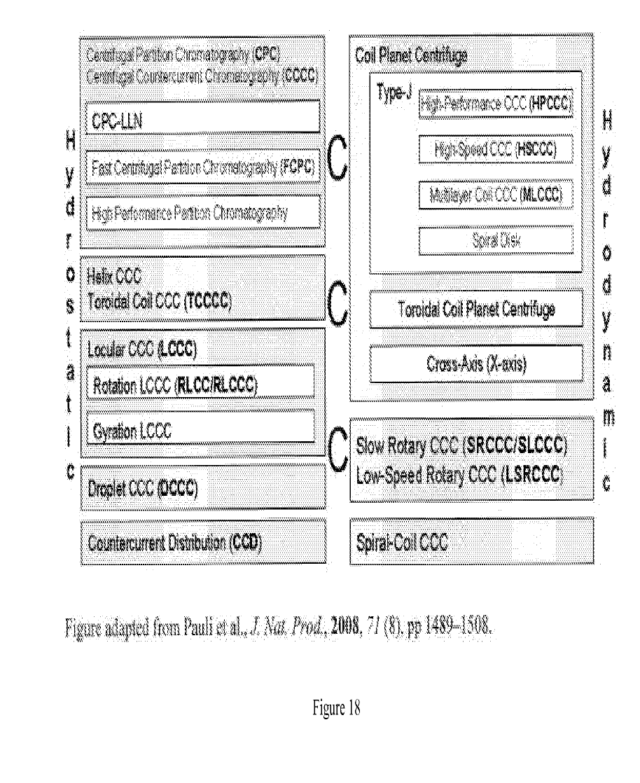

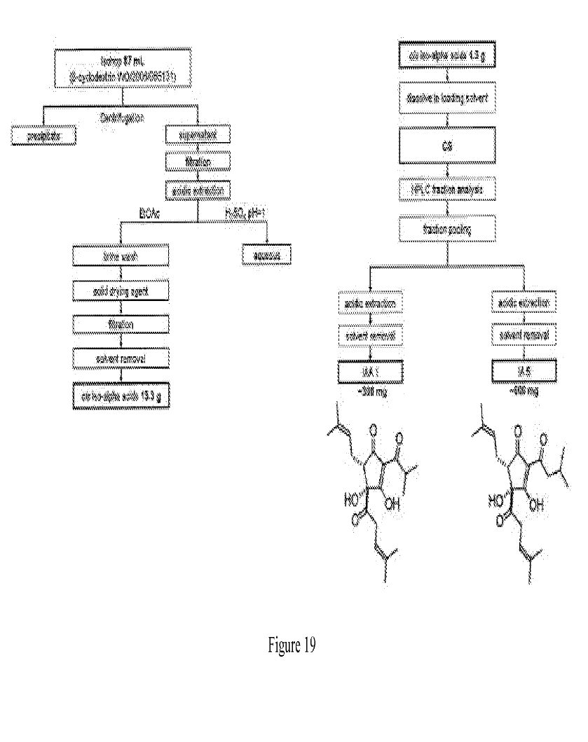

A mixture of cis iso-alpha acid (IAA) congeners (free acid) was obtained from an aqueous solution of the potassium salt of a mixture of cis and trans IAA congeners. This solution, sold as Isohop®, was kindly provided by John I Haas, Yakima, WA. A slightly modified procedure as described in WO/2006/065131 was employed in order to obtain a mixture of cis iso-alpha acid (IAA) congeners. Specifically, centrifugation was used to remove uncomplexed, excess β-cyclodextrin. Furthermore, it was discovered that a sufficient amount of H₂SO₄ (aq) pH=1 and extraction into EtOAc, is critical in order to minimize the formation of difficult emulsions. Following the dilution of the Isohop® with water, it was treated with β-cyclodextrin according to the procedure described in WO/2006/065131. The aforementioned modifications to this procedure were implemented. According to this procedure (FIG. 19) a relatively high yield of a mixture of cis iso-alpha acid (IAA) congeners (free acid) 13.3 g, was obtained from 87 mL of Isohop®.

The solvent system for the HSCCC purification of individual cis IAA congeners, from a mixture of cis IAA congeners, was made by equilibrating 4000 mL of hexane, 14000 mL of water, 240 mL of concentrated ammonium hydroxide ([14.5 M] 71% H₂O), and 325 mL of glacial acetic acid ([17.5 M]) in a separatory funnel, the pH was checked and found to be 4.92. The organic “upper” phase was separately collected from the aqueous “lower” phase. The “upper” phase was used as the stationary phase, and henceforth used to initially charge the 820 mL HSCCC coils. Upon complete coil filling, the HSCCC was spun at 700 RPM, and the upper phase was pumped at 4 mL/min in a ‘tail-to-head’ elution also referred to as an “ascending” or “normal phase” elution mode. When the upper phase eluted from head of the coils, the equilibration was complete; pumping the “upper” phase at a flow rate of 4 mL/min, was continued.

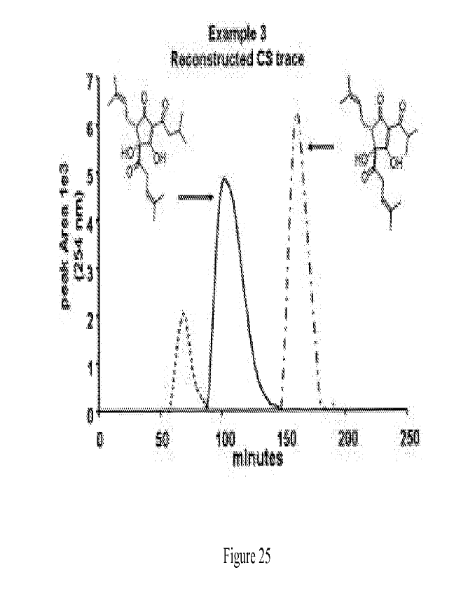

1313 mg of the cis IAA free acid was dissolved in 50 mL of the upper phase, and 50 mL of the lower phase. The sample was then injected into an empty 100 mL sample loop. At this time the HSCCC was fully equilibrated and the sample-loop was injected into the coils. The UV detector began to monitor the eluent coming out of the HSCCC coils immediately following the sample-loop injection. The flow rate of the “upper” mobile phase continued at 4 mL/min and fractions were collected in 80 mL volumes after the first 200 minutes. After 600 minutes, the “upper” phase was no longer used as the mobile phase; instead additional “lower” stationary phase was pumped into the coils at a flow rate of 4 ml/min. The replacement of the “upper” phase with the “lower” phase marks the beginning of the elution-extrusion method; the total run time was 825 minutes. Following 825 minutes after the sample-loop injection, all of the collected fractions were analyzed using HPLC in order to determine which fractions contained homogenous solutions of purified cis IAA congeners. Upon completing the HPLC analysis of each fraction, a CS trace was reconstructed by plotting the peak area of each of the purified cis-IAA congeners versus time (minutes) or several other parameters e.g., volume (mL) or “K”, as shown in FIG. 25. Based upon the homogeneity of the collected fractions, they were pooled accordingly and extracted into hexanes via an acidic aqueous extraction as described in FIG. 19. This final acidic extraction is conducted in order to remove any residual water form the fractions. The hexanes were removed in vacuo to yield highly pure cis IAA congeners. This procedure rendered 338 mg of IA 1, and 607 mg of IA5 5, respectively, in >95% purity as determined by HPLC.

### Example 7

### Countercurrent Separation (CS) Method for the Purification of the Tetrahydro Iso-Alpha Acids (THIAA)

Hops (Humulus Lupulus L.) are well-known plants that have been used in the brewing of beer for over 1500 years. Various modified extracts of the hop cone are currently used in contemporary beer brewing for their bitter taste and foam stabilizing properties. Among these extracts, the tetrahydro iso-alpha acids (THIAAs) have been recently reported to exert significant anti-inflammatory effects in a wide range of enzymatic and cellular assays. For this reason, THIAAs have been successfully incorporated in several medical foods that support the nutritional requirements of individuals with inflammatory related health conditions.

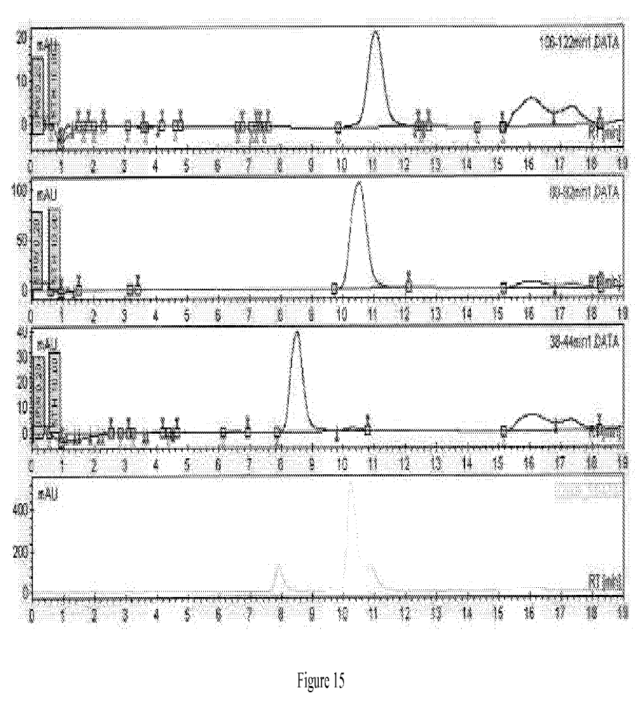

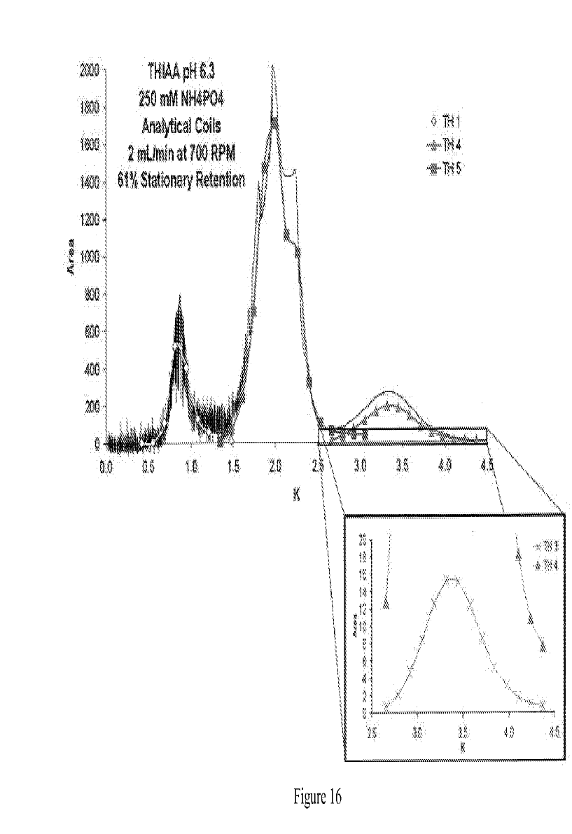

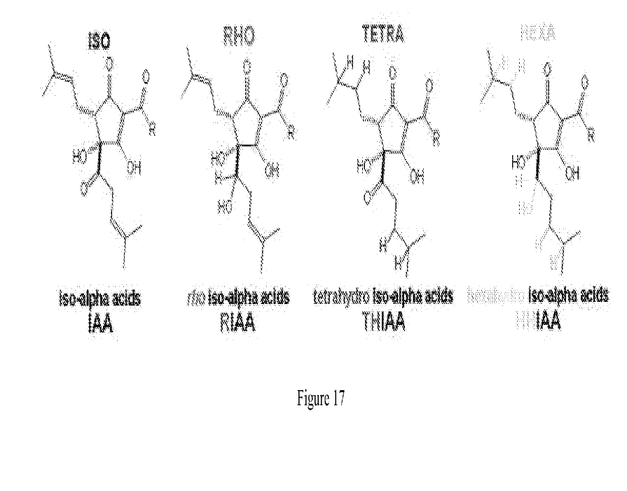

The THIAA extract consists of a well-defined yet complex mixture of closely related branched short-chain fatty-acid derived congeners and diastereomers. It has been reported that the predominant constituents of THIAA are the cis n- the cis co- and the trans-congeners as shown in FIG. 17 and listed in Table 10. We sought to develop a reliable and efficient method for the rapid preparative purification of these individual congeners in order to determine their relative differences in various models of inflammation.

We focused on a developing a counter current separation method that would enable us to obtain gram quantities of each of the major congeners in high purity (>99%). We began by investigating the partitioning of THIAA in a variety of solvent systems according to the so-called ‘shake-flask’ protocol. During the evaluation of a wide variety of solvent systems we discovered that pH, the type of buffer and the concentration of buffer relative to the concentration of THIAA, significantly effects the partitioning of the THIAA congeners.

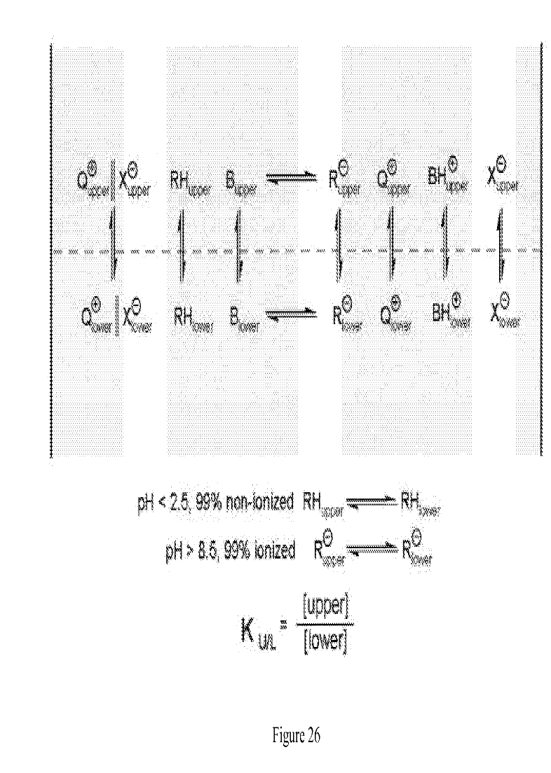

As shown in FIG. 26, complex equilibria between a specific congener of iso alpha acid (or reduced iso-alpha acids) and any salt or buffers present in the solvent system. The salient feature of this depiction is the importance of the partitioning of the congener when it is non-ionized and ionized (RH). For this reason the pKa of the congener which is an inherent property will greatly affect the overall partitioning. Furthermore the exact role of the salts and buffers will also influence the extent of ionization and by virtue of ion-pairing with the conjugate base of the congener, the partitioning of the ionized form of the congener as well.

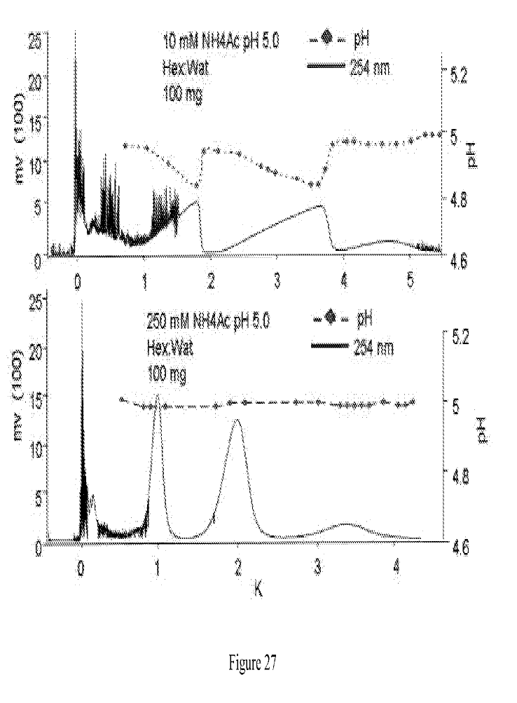

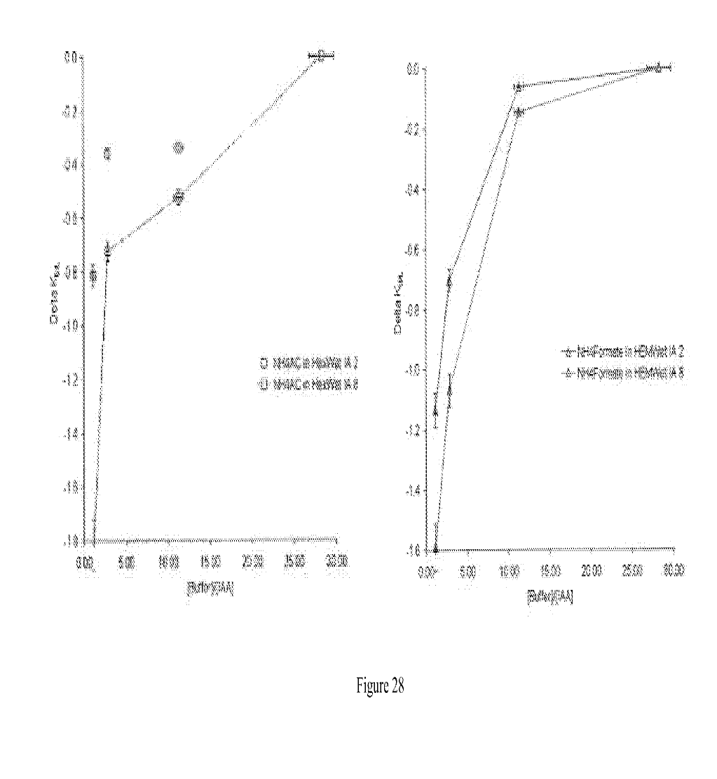

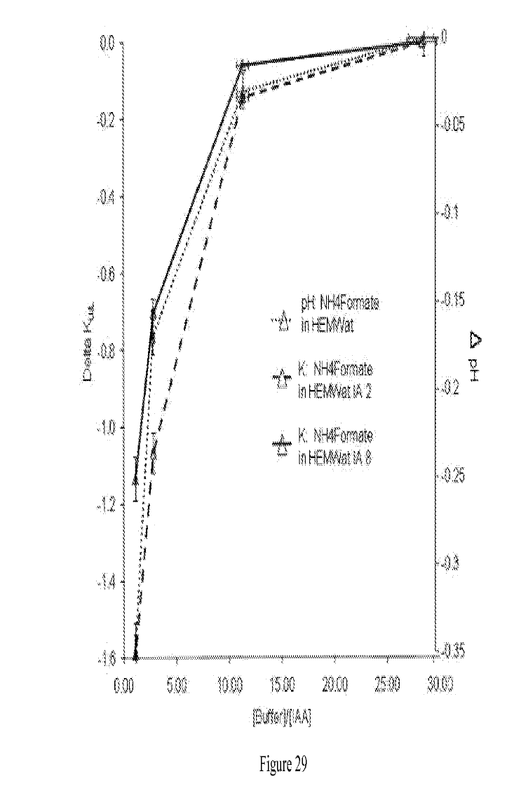

As shown in FIG. 27, the concentration of buffer greatly impacts the partitioning of IAA congeners IA1, IA5 and IA4. The importance of the buffer stoichiometry and its effects on K_{U/L} has been investigated (see FIG. 28). An illustration of the effects of the stoichiometry between the buffer and two congeners is shown FIG. 28. These two graphs (each showing the relationship between ΔK_{U/L} for two IAA congeners) demonstrate the importance of the amount of buffer relative the amount of IAA. The graph in FIG. 29 addresses how the amount of buffer affects the pH in the two solvent systems SS1 (HexWat) and SS2 and (Hemwat) for two IAA congeners. Based upon these discoveries we were able to optimize a CS method that enables the purification of various THIAA with high purity, high-yield and minimal time.

Experimental

HPLC analyses were performed using a Shimadzu HPLC system (Shimadzu Scientific Instruments, Inc., Columbia, Md.). The system consists of a DGU 14A solvent degasser, LC-10AD solvent pumps (3), SPD-M10ADVP photodiode array detector monitoring at 254 nm, SIL-10ADVP auto injector, SCL10AVP system controller and a CTO-10AVP column oven operating at 40° C. This system is controlled using Class VP 7.3 sp1 software. A Phenomenex Gemini NX C18 column (Torrance, Calif.), 4.6×250 mm, 3 μm particle size was used for monitoring HSCCC fraction homogeneity. The mobile phase consisted of 20 mM ammonium acetate to an apparent pH of 9.50 with ammonia (solvent A) and acetonitrile/methanol 60/40 (v/v) (solvent B). The flow-rate was set at 1 ml/min and isocratic elution (42% solvent B) for 15.5 min followed by a wash (95% solvent B) and re-equilibration, the total method length was 23 minutes.

All HSCCC experimentation was conducted on a CCC-1000 J-type three-coiled planetary motion HSCCCC (Pharma-Tech Research Corp., Baltimore, Md., USA). The rotation radius is 7.5 cm, and 3×108 mL PTFE Teflon coils with an inner diameter of 1.6 mm, an outer diameter of 2.7 mm, and beta values of 0.47 to 0.73 for all coils. This was coupled with Shimadzu (Kyoto, Japan) liquid chromatography instrumentation: LC-20AT pump, DGU-20A₅ degasser, CBM-20A controller, and SPD-10AV UV detector. A preparative Rheodyne (Rohnert Park, Calif. USA) 3725i-038 injector was in line with a 3.8 mL sample loop (changed to 10 mL for entry 7). The sample was monitored at 254 nm and 314 nm during the course of the run.

Reagents and Materials

A THIAA standard, consisting of the cis and trans diastereomers of a mixture of predominantly n-, co- and ad-congeners (99% DCHA salt) was purchased from the American Society of Brewing Chemists, (ASBC, St. Paul, Minn.) and used throughout this study. A commercial preparation of a cis THIAA mixture consisting predominantly of n-, co- and ad-congeners was also used for this study.

Mobile phases used for all HPLC analyses were supplied by either Burdick & Jackson (B&J ACS/HPLC grade 99.9%) or EMD (OMNISOLV, 99.9% high purity HPLC grade) and used directly. The ethanol was denatured (formula 3A). Water was obtained from a Barnstead Nanopure Infinity Ultrapure system maintained in our laboratories. The various chemicals used as buffers in the mobile phases were purchased from Aldrich Chemical and used without further purification.

Partition Coefficient (K_{U/L}) Shake-Flask Measurement.

The measurement of the partition coefficient K_{U/L}, the ratio of the upper phase concentration (U) to the lower phase concentration (L), was performed according to a protocol stated elsewhere in this application. Briefly, 5 uL of a stock solution of THIAA standard (400 mg/mL) in MeOH was added to a solvent system (20 mL) comprised of two immiscible phases of approximately equal volume. Following the addition of analyte, the bi-phasic mixture was vortexed and the settling time was recorded. An aliquot from each phase was transferred directly into an HPLC sample vial and submitted for HPLC analysis (by the previously described methods) using a 10 uL injection volume. The upper and lower peak areas, respectively, were used to calculate K_{U/L} for individual THIAA congeners in a variety of solvent systems. A representative HPLC chromatogram using a method we devised is shown in FIG. 22. This method allowed us to determine the concentrations of each THIAA congener in each phase with high reproducibility (<5% CV).

Two mobile phases, A and B, were used throughout this investigation. Solvent A consisted of an aqueous buffer; solvent B consisted of acetonitrile and/or an alcohol. A summary of mobile phase composition, as well as details describing the methods, e.g., flow rate, pH, etc., are listed in Table 9. Each entry in Table 9 was analyzed using a stock solution of Redihop® (0.1 mg/mL in MeOH) to measure the partitioning values by the previously mentioned shake flask procedure.

HSCCC Separation

K_{U/L} values for THIAA were determined in a variety of HEMWat solvent systems according to the shake-flask partitioning assay. Two solvent systems, referred to as A and B, provided K values within the range 1.5-2.5 and with significant differences between THIAA congeners (ΔK). For these reasons solvent systems A and B were selected for further development on the HSCCC (Table 9). Compared to descending elution, an ascending elution method provided significantly higher stationary phase retention; hence an ascending elution method was the preferred method of elution for all THIAA HSCCC purification trials. The entries in Table 10 correspond to seven trial THIAA HSCCC purifications; entries 1-3 were conducted using solvent system A; entries 4-7 used solvent system B. Because of the difference in THIAA concentration between the shake-flask assay (0.1 mg/mL, 0.273 mM) and the HSCCC sample loop (100 mg/mL; 273 mM), aqueous phase buffer concentrations of 1.2 M (entries 1-3, Table 10) and 555 mM (entries 4-7, Table 10) were employed for the HSCCC trials. Entry 3 in Table 10 uses a bi-phasic sample-loop injection (solvent C, Table 9) consisting of 1.9 mL of upper phase (200 mg/mL THIAA, 0.55M) and 1.9 mL of lower phase (6.5M NH₄HAc aq.). A switching volume was employed in entries 2, 6 and 7 in order to assess the elution-extrusion protocol as reported by Pauli et al.

Entries 4 & 5 Table 10, examine the effect of increasing the pH for solvent system B on the resolution between THIAA congeners. We reasoned that increased pH leads to a greater concentration of the ionized form of the THIAA (conjugate base) thus resulting in greater amounts of THIAA retained in the aqueous stationary phase i.e., increased retention volume (Vᵣ). According to a model, that successfully predicts the effect of the pH on THIAA resolution for solvent system B, pH 6.8 (entry 5, Table 10) provides an optimum resolution for this solvent system and increasing the pH beyond this point provides negligible improvement in resolution with significantly greater Vᵣ.

Results

According to entry 6, Table 10, 380 mg, 1.0 mmole of THIAA was loaded onto the coils and nearly three times as much, 1000 mg, 2.7 mmole, was loaded in the following entry (7). Despite the submission of nearly three times the amount of THIAA in entry 6, the Vr for the THIAA congeners in entry 7 remained unchanged. The major difference between the separations resulting from entries 6 & 7 is the degree of tailing evident in the larger sample loading, entry 7. In this case, the purity of the later eluting THIAA congeners is diminished relative to entry 6.

TABLE 9
Solvent-system compositions used for the evaluation of countercurrent separations
(CS); with the exception of pH, all values are in units of mL

| | | | | | | NH₄OH | H₃PO₄ | HCl | buffer |
|---|---|---|---|---|---|---|---|---|---|
| solvent | | | | | | [14.5M] | [14.8M] | [12.1M] | molarity |
| system | pH | n-C₆H₁₄ | EtOAc | CH₃OH | H₂O | 71% H₂O | 15% H₂O | 63% H₂O | [M] |
| A_{HSCCC} | 5.30 | 700 | 300 | 500 | 400 | 30 | 0 | 20 | 1.20¹ |
| B_{HSCCC} | 6.55 | 1000 | 0 | 0 | 900 | 60 | 35 | 0 | 0.55² |
| B′_{HSCCC} | 6.78 | 1000 | 0 | 0 | 900 | 65 | 35 | 0 | 0.54³ |
| Cₛₐₘₚₗₑ₋ₗₒₒₚ | 5.42 | 35 | 15 | 25 | 10 | 6 | 0 | 3 | 6.46⁴ |

¹Total volume of H₂O ~ 434 mL.
²Total volume of H₂O ~ 945 mL.
³Total volume of H₂O ~ 951 mL
⁴Total volume of H₂O ~ 16.2 mL

TABLE 10
Results for various trial CS puri-
fications of THIAA raw material⁵

| trial | sol- vent sys- tem | switch- ing vol- ume (mL) | $\begin{matrix}\begin{matrix}Partition Coefficient \\ {K}_{HSCCC} = 1 +\end{matrix} \\ \frac{{V}_{R} - 320 mL}{{V}_{S}} \\ TH 1 TH 4 TH 5\end{matrix}$ | | | $\begin{matrix}Resolution \\ 2 \frac{{( {V}_{R} )}_{B} - {( {V}_{R} )}_{A}}{{W}_{A} + {W}_{B}} \\ TH 1 \text{-} TH 1 \text{-} TH 4 \text{-} \\ TH 4 TH 5 TH 5\end{matrix}$ | | |
|---|---|---|---|---|---|---|---|---|
| 1 | A | | 2.0 | 1.0 | 1.4 | 1.9 | 1.0 | 0.7 |
| 2 | A | 320 | 2.0 | 1.0 | 1.4 | 1.8 | 0.6 | 0.6 |
| 3⁶ | A | | 2.1 | 1.1 | 1.4 | 1.8 | 0.9 | 0.6 |
| 4 | B′ | | 1.7 | 0.6 | 0.9 | 2.0 | 1.2 | 0.5 |
| 5 | B | | 2.5 | 0.8 | 1.3 | 2.7 | 1.5 | 0.8 |
| 6 | B | 600 | 2.7 | 0.8 | 1.3 | 3.9 | 2.0 | 0.8 |
| 7⁷ | B | 520 | 2.6 | 0.8 | 1.5 | 2.8 | 1.0 | 0.8 |

⁵Unless otherwise noted, an ascending mode of elution was employed using an HSCCC instrument (PharmaTech Research CCC-1000) at 680 rpm with a 4 mL/min flow-rate and a sample-loop injection volume of 3.8 mL for all entries.
⁶A bi-phasic sample-loop injection was used; 1.9 mL solvent system C, Table 9, in a sample-loop volume of 3.8 mL.
⁷1.0 g of raw material (100 mg/mL) was loaded onto HSCCC using a 10 mL sample loop.

TABLE 11
Results for the recovery and % homogeneity of material

| | Recovered cisTHIAA⁸ mg (% homogeneity) | | |
|---|---|---|---|
| trial | TH1 | TH3,4 | TH5 |
| 1 | 14(94) | 11(99) | 180(98) |
| 2 | 10(90) | 11(100) | 174(98) |
| 3 | 13(95) | 10(100) | 186(97) |
| 4 | 16(90) | 10(100) | 173(99) |
| 5 | 10(84) | 10(100) | 170(100) |
| 6 | 15(86) | 11(100) | 179(98) |
| 7 | 0 | 25(100) | 417(99) |

⁸Based upon 380 mg sample loading, the % area from HPLC fraction analysis and a total cisTHIAA recovery of 61% according to trial using 1.021 g of cisTHIAA

### CONCLUSION

The results for this experiment are summarized in Table 11. In comparison of the seven variations of THIAA purification by CS, the best resolution was obtained by optimizing the solvent system composition, the amount of material loaded and the pH. Introducing the sample through a biphasic injection of concentrated buffer, as seen in entry 3, produced minimal gains over entry 1. Similarly optimizing EECCC, as seen in entry 6, produced minimal gains in resolution over entry 5, however the EECCC did serve to regenerate the stationary phase for subsequent runs. Optimizing the amount of sample loaded to prevent tailing, and selecting an appropriate solvent system (pH inclusive) to obtain the maximum resolution of the desired component(s) proves to be the best overall method. Thus, entry 6 provides the most useful method to obtain gram quantities of high purity THIAA congeners in a minimal amount of time.

The invention now having been fully described, it will be apparent to one of ordinary skill in the art that many changes and modifications can be made thereto without departing from the spirit or scope of the appended claims.

1. A method for isolating a congener in substantially pure form from a mixture of congeners comprising the steps of:
a. selecting a congener to be isolated;
b. dissolving the mixture of congeners in a bi-phasic solvent system specific to the selected congener to be isolated, wherein said bi-phasic solvent system has a partition coefficient from about 0.5 to 5.0;
c. subjecting the mixture of congeners dissolved in the bi-phasic solvent system to a counter current chromatography; and
d. isolating the selected congener;
wherein said congener is selected from the group consisting of substituted cyclohexa-2,4-dienones, substituted cyclohexane-1,3,5-triones, substituted cyclopent-2-en-1-ones, and substituted 1,3-cyclopentadiones.

2. The method of claim 1, wherein the substituted cyclohexa-2,4-dienone is selected from the group consisting of (6R)-3,5,6-trihydroxy-2-(3-methylbutanoyl)-4,6-bis(3-methylbut-2-en-1-yl)cyclohexa-2,4-dien-1-one, (6R)-3,5,6-trihydroxy-4,6-bis(3-methylbut-2-en-1-yl)-2-(2-methylpropanoyl)cyclohexa-2,4-dien-1-one, and (6R)-3,5,6-trihydroxy-2-(2-methylbutanoyl)-4,6-bis(3-methylbut-2-en-1-yl)cyclohexa-2,4-dien-1-one.

3. The method of claim 1, wherein the substituted cyclohexane-1,3,5-trione is selected from the group consisting of substituted cyclohexane-1,3,5-triones wherein said composition is enriched for one or more compounds selected from the group consisting of 3,5-dihydroxy-2-(3-methylbutanoyl)-4,6,6-tris(3-methylbut-2-en-1-yl)cyclohexa-2,4-dien-1-one, 3,5-dihydroxy-4,6,6-tris(3-methylbut-2-en-1-yl)-2-(2-methylpropanoyl)cyclohexa-2,4-dien-1-one, and 3,5-dihydroxy-2-(2-methylbutanoyl)-4,6,6-tris(3-methylbut-2-en-1-yl)cyclohexa-2,4-dien-1-one.

4. The method of claim 1, wherein the substituted cyclopent-2-en-1-one is selected from the group consisting of (4R,5S)-4-hydroxy-3-methyl-2-(3-methylbutanoyl)-5-(3-methylbut-2-en-1-yl)-4-(4-methylpent-3-enoyl)cyclopent-2-en-1-one, (4S,5S)-4-hydroxy-3-methyl-2-(3-methylbutanoyl)-5-(3-methylbut-2-en-1-yl)-4-(4-methylpent-3-enoyl)cyclopent-2-en-1-one, (4S,5R)-4-hydroxy-3-methyl-2-(3-methylbutanoyl)-5-(3-methylbut-2-en-1-yl)-4-(4-methylpent-3-enoyl)cyclopent-2-en-1-one, (4R,5R)-4-hydroxy-3-methyl-2-(3-methylbutanoyl)-5-(3-methylbut-2-en-1-yl)-4-(4-methylpent-3-enoyl)cyclopent-2-en-1-one, (4R,5S)-4-hydroxy-3-methyl-5-(3-methylbut-2-en-1-yl)-4-(4-methylpent-3-enoyl)-2-(2-methylpropanoyl)cyclopent-2-en-1-one, (4S,5S)-4-hydroxy-3-methyl-5-(3-methylbut-2-en-1-yl)-4-(4-methylpent-3-enoyl)-2-(2-methylpropanoyl)cyclopent-2-en-1-one, (4S,5R)-4-hydroxy-3-methyl-5-(3-methylbut-2-en-1-yl)-4-(4-methylpent-3-enoyl)-2-(2-methylpropanoyl)cyclopent-2-en-1-one, (4R,5R)-4-hydroxy-3-methyl-5-(3-methylbut-2-en-1-yl)-4-(4-methylpent-3-enoyl)-2-(2-methylpropanoyl)cyclopent-2-en-1-one, (4R,5S)-4-hydroxy-3-methyl-2-(2-methylbutanoyl)-5-(3-methylbut-2-en-1-yl)-4-(4-methylpent-3-enoyl)cyclopent-2-en-1-one, (4S,5S)-4-hydroxy-3-methyl-2-(2-methylbutanoyl)-5-(3-methylbut-2-en-1-yl)-4-(4-methylpent-3-enoyl)cyclopent-2-en-1-one, (4S,5R)-4-hydroxy-3-methyl-2-(2-methylbutanoyl)-5-(3-methylbut-2-en-1-yl)-4-(4-methylpent-3-enoyl)cyclopent-2-en-1-one, and (4R,5R)-4-hydroxy-3-methyl-2-(2-methylbutanoyl)-5-(3-methylbut-2-en-1-yl)-4-(4-methylpent-3-enoyl)cyclopent-2-en-1-one.

5. The method of claim 1, wherein the substituted 1,3-cyclopentadione is selected from the group consisting of (4S,5S)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5S)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5S)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5S)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4R,5R)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5R)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5R)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5R)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4S,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4S,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4R,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5R)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4S,5R)-3,4-dihydroxy-4-[(1S)-hydroxy-4-methylpent-3-en-1-yl]-2-(2-methylpropanoyl)-5-(3-methylbut-2-en-1-yl)cyclopent-2-en-1-one; (4S,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4S,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4S,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4R,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4S,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; and to 99.9 percent by weight of (4S,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one.

6. The method of claim 1, wherein the substituted 1,3-cyclopentadione is selected from the group consisting of selected from the group consisting of (4R,5S)-3,4-dihydroxy-2-(3-methylbutanoyl)-5-(3-methylbutyl)-4-(4-methylpentanoyl)cyclopent-2-en-1-one, (4S,5S)-3,4-dihydroxy-2-(3-methylbutanoyl)-5-(3-methylbutyl)-4-(4-methylpentanoyl)cyclopent-2-en-1-one, (4S,5R)-3,4-dihydroxy-2-(3-methylbutanoyl)-5-(3-methylbutyl)-4-(4-methylpentanoyl)cyclopent-2-en-1-one, (4R,5R)-3,4-dihydroxy-2-(3-methylbutanoyl)-5-(3-methylbutyl)-4-(4-methylpentanoyl)cyclopent-2-en-1-one, (4R,5S)-3,4-dihydroxy-5-(3-methylbutyl)-4-(4-methylpentanoyl)-2-(3-methylpropanoyl)cyclopent-2-en-1-one, (4S,5S)-3,4-dihydroxy-5-(3-methylbutyl)-4-(4-methylpentanoyl)-2-(3-methylpropanoyl)cyclopent-2-en-1-one, (4S,5R)-3,4-dihydroxy-5-(3-methylbutyl)-4-(4-methylpentanoyl)-2-(3-methylpropanoyl)cyclopent-2-en-1-one, (4R,5R)-3,4-dihydroxy-5-(3-methylbutyl)-4-(4-methylpentanoyl)-2-(3-methylpropanoyl)cyclopent-2-en-1-one, (4R,5S)-3,4-dihydroxy-2-(2-methylbutanoyl)-5-(3-methylbutyl)-4-(4-methylpentanoyl)cyclopent-2-en-1-one, (4S,5S)-3,4-dihydroxy-2-(2-methylbutanoyl)-5-(3-methylbutyl)-4-(4-methylpentanoyl)cyclopent-2-en-1-one, (4S,5R)-3,4-dihydroxy-2-(2-methylbutanoyl)-5-(3-methylbutyl)-4-(4-methylpentanoyl)cyclopent-2-en-1-one, and (4R,5R)-3,4-dihydroxy-2-(2-methylbutanoyl)-5-(3-methylbutyl)-4-(4-methylpentanoyl)cyclopent-2-en-1-one.

7. The method of claim 1, wherein the substituted 1,3-cyclopentadione is selected from the group consisting of selected from the group consisting of (4S,5S)-3,4-dihydroxy-4-[(1S)-1-hydroxy-4-methylpentyl]-2-(3-methylbutanoyl)-5-(3-methylbutyl)cyclopent-2-en-1-one, (4S,5S)-3,4-dihydroxy-4-[(1R)-1-hydroxy-4-methylpentyl]-2-(3-methylbutanoyl)-5-(3-methylbutyl)cyclopent-2-en-1-one, (4R,5S)-3,4-dihydroxy-4-[(1R)-1-hydroxy-4-methylpentyl]-2-(3-methylbutanoyl)-5-(3-methylbutyl)cyclopent-2-en-1-one, (4R,5S)-3,4-dihydroxy-4-[(1S)-1-hydroxy-4-methylpentyl]-2-(3-methylbutanoyl)-5-(3-methylbutyl)cyclopent-2-en-1-one, (4R,5R)-3,4-dihydroxy-4-[(1R)-1-hydroxy-4-methylpentyl]-2-(3-methylbutanoyl)-5-(3-methylbutyl)cyclopent-2-en-1-one, (4R,5R)-3,4-dihydroxy-4-[(1S)-1-hydroxy-4-methylpentyl]-2-(3-methylbutanoyl)-5-(3-methylbutyl)cyclopent-2-en-1-one, (4S,5R)-3,4-dihydroxy-4-[(1S)-1-hydroxy-4-methylpentyl]-2-(3-methylbutanoyl)-5-(3-methylbutyl)cyclopent-2-en-1-one, (4S,5R)-3,4-dihydroxy-4-[(1R)-1-hydroxy-4-methylpentyl]-2-(3-methylbutanoyl)-5-(3-methylbutyl)cyclopent-2-en-1-one, (4S,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4S,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4R,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5R)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4S,5R)-3,4-dihydroxy-4-[(1S)-hydroxy-4-methylpent-3-en-1-yl]-2-(2-methylpropanoyl)-5-(3-methylbut-2-en-1-yl)cyclopent-2-en-1-one, (4S,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4S,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4S,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4R,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4S,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, and (4S,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one.

8. The method according to claim 1, wherein the solvent for the bi-phasic system is selected from the group comprising water, water containing a buffering agent, water containing a soluble polymer, a pentane, hexane, heptane, octane, methyl acetate, ethyl acetate, propyl acetate, butyl acetate, tert-butyl acetate, methanol, ethanol, propanol, iso propanol, butanol, tert butanol, dimethyl formamide, dimethyl sulfoxide, dichloromethane, chloroform; and acetone, or any combination thereof.

9. The method of claim 1, wherein the bi-phasic solvent system has a partition coefficient between 0.6 and 3.0.

10. The method of claim 1, wherein the bi-phasic solvent system has a partition coefficient between 0.7 and 1.5.

11. The method of claim 1, wherein the counter current chromatography is performed at a temperature of about 20° C. to about 30° C.

12. The method of claim 1, wherein the counter current chromatography is performed at ambient temperature.

13. A composition comprising a substantially pure congener or a pharmaceutically acceptable salt thereof, wherein said congener is obtained from a mixture of congeners comprising the steps of:
a. selecting a congener to be isolated;
b. dissolving the mixture of congeners in a bi-phasic solvent system specific to the selected congener to be isolated, wherein said bi-phasic solvent system has a partition coefficient from about 0.5 to 5.0;
c. subjecting the mixture of congeners dissolved in the bi-phasic solvent system to a counter current chromatography; and
d. isolating the selected congener;
wherein said congener is selected from the group consisting of substituted cyclohexa-2,4-dienones, substituted cyclohexane-1,3,5-triones, substituted cyclopent-2-en-1-ones, and substituted 1,3-cyclopentadiones; and
wherein said composition further comprises a pharmaceutically acceptable excipient.

14. The composition of claim 13, wherein the substituted cyclohexa-2,4-dienone is selected from the group consisting of (6R)-3,5,6-trihydroxy-2-(3-methylbutanoyl)-4,6-bis(3-methylbut-2-en-1-yl)cyclohexa-2,4-dien-1-one, (6R)-3,5,6-trihydroxy-4,6-bis(3-methylbut-2-en-1-yl)-2-(2-methylpropanoyl)cyclohexa-2,4-dien-1-one, and (6R)-3,5,6-trihydroxy-2-(2-methylbutanoyl)-4,6-bis(3-methylbut-2-en-1-yl)cyclohexa-2,4-dien-1-one.

15. The composition of claim 13, wherein the substituted cyclohexane-1,3,5-trione is selected from the group consisting of substituted cyclohexane-1,3,5-triones wherein said composition is enriched for one or more compounds selected from the group consisting of 3,5-dihydroxy-2-(3-methylbutanoyl)-4,6,6-tris(3-methylbut-2-en-1-yl)cyclohexa-2,4-dien-1-one, 3,5-dihydroxy-4,6,6-tris(3-methylbut-2-en-1-yl)-2-(2-methylpropanoyl)cyclohexa-2,4-dien-1-one, and 3,5-dihydroxy-2-(2-methylbutanoyl)-4,6,6-tris(3-methylbut-2-en-1-yl)cyclohexa-2,4-dien-1-one.

16. The composition of claim 13, wherein the substituted cyclopent-2-en-1-one is selected from the group consisting of (4R,5S)-4-hydroxy-3-methyl-2-(3-methylbutanoyl)-5-(3-methylbut-2-en-1-yl)-4-(4-methylpent-3-enoyl)cyclopent-2-en-1-one, (4S,5S)-4-hydroxy-3-methyl-2-(3-methylbutanoyl)-5-(3-methylbut-2-en-1-yl)-4-(4-methylpent-3-enoyl)cyclopent-2-en-1-one, (4S,5R)-4-hydroxy-3-methyl-2-(3-methylbutanoyl)-5-(3-methylbut-2-en-1-yl)-4-(4-methylpent-3-enoyl)cyclopent-2-en-1-one, (4R,5R)-4-hydroxy-3-methyl-2-(3-methylbutanoyl)-5-(3-methylbut-2-en-1-yl)-4-(4-methylpent-3-enoyl)cyclopent-2-en-1-one, (4R,5S)-4-hydroxy-3-methyl-5-(3-methylbut-2-en-1-yl)-4-(4-methylpent-3-enoyl)-2-(2-methylpropanoyl)cyclopent-2-en-1-one, (4S,5S)-4-hydroxy-3-methyl-5-(3-methylbut-2-en-1-yl)-4-(4-methylpent-3-enoyl)-2-(2-methylpropanoyl)cyclopent-2-en-1-one, (4S,5R)-4-hydroxy-3-methyl-5-(3-methylbut-2-en-1-yl)-4-(4-methylpent-3-enoyl)-2-(2-methylpropanoyl)cyclopent-2-en-1-one, (4R,5R)-4-hydroxy-3-methyl-5-(3-methylbut-2-en-1-yl)-4-(4-methylpent-3-enoyl)-2-(2-methylpropanoyl)cyclopent-2-en-1-one, (4R,5S)-4-hydroxy-3-methyl-2-(2-methylbutanoyl)-5-(3-methylbut-2-en-1-yl)-4-(4-methylpent-3-enoyl)cyclopent-2-en-1-one, (4S,5S)-4-hydroxy-3-methyl-2-(2-methylbutanoyl)-5-(3-methylbut-2-en-1-yl)-4-(4-methylpent-3-enoyl)cyclopent-2-en-1-one, (4S,5R)-4-hydroxy-3-methyl-2-(2-methylbutanoyl)-5-(3-methylbut-2-en-1-yl)-4-(4-methylpent-3-enoyl)cyclopent-2-en-1-one, and (4R,5R)-4-hydroxy-3-methyl-2-(2-methylbutanoyl)-5-(3-methylbut-2-en-1-yl)-4-(4-methylpent-3-enoyl)cyclopent-2-en-1-one.

17. The composition of claim 13, wherein the substituted 1,3-cyclopentadione is selected from the group consisting of (4S,5S)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5S)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5S)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5S)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4R,5R)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5R)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5R)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5R)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4S,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4S,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4R,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5R)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4S,5R)-3,4-dihydroxy-4-[(1S)-hydroxy-4-methylpent-3-en-1-yl]-2-(2-methylpropanoyl)-5-(3-methylbut-2-en-1-yl)cyclopent-2-en-1-one; (4S,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4S,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4S,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4R,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4S,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; and to 99.9 percent by weight of (4S,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one.

18. The composition of claim 13, wherein the substituted 1,3-cyclopentadione is selected from the group consisting of selected from the group consisting of (4R,5S)-3,4-dihydroxy-2-(3-methylbutanoyl)-5-(3-methylbutyl)-4-(4-methylpentanoyl)cyclopent-2-en-1-one, (4S,5S)-3,4-dihydroxy-2-(3-methylbutanoyl)-5-(3-methylbutyl)-4-(4-methylpentanoyl)cyclopent-2-en-1-one, (4S,5R)-3,4-dihydroxy-2-(3-methylbutanoyl)-5-(3-methylbutyl)-4-(4-methylpentanoyl)cyclopent-2-en-1-one, (4R,5R)-3,4-dihydroxy-2-(3-methylbutanoyl)-5-(3-methylbutyl)-4-(4-methylpentanoyl)cyclopent-2-en-1-one, (4R,5S)-3,4-dihydroxy-5-(3-methylbutyl)-4-(4-methylpentanoyl)-2-(3-methylpropanoyl)cyclopent-2-en-1-one, (4S,5S)-3,4-dihydroxy-5-(3-methylbutyl)-4-(4-methylpentanoyl)-2-(3-methylpropanoyl)cyclopent-2-en-1-one, (4S,5R)-3,4-dihydroxy-5-(3-methylbutyl)-4-(4-methylpentanoyl)-2-(3-methylpropanoyl)cyclopent-2-en-1-one, (4R,5R)-3,4-dihydroxy-5-(3-methylbutyl)-4-(4-methylpentanoyl)-2-(3-methylpropanoyl)cyclopent-2-en-1-one, (4R,5S)-3,4-dihydroxy-2-(2-methylbutanoyl)-5-(3-methylbutyl)-4-(4-methylpentanoyl)cyclopent-2-en-1-one, (4S,5S)-3,4-dihydroxy-2-(2-methylbutanoyl)-5-(3-methylbutyl)-4-(4-methylpentanoyl)cyclopent-2-en-1-one, (4S,5R)-3,4-dihydroxy-2-(2-methylbutanoyl)-5-(3-methylbutyl)-4-(4-methylpentanoyl)cyclopent-2-en-1-one, and (4R,5R)-3,4-dihydroxy-2-(2-methylbutanoyl)-5-(3-methylbutyl)-4-(4-methylpentanoyl)cyclopent-2-en-1-one.

19. The composition of claim 13, wherein the substituted 1,3-cyclopentadione is selected from the group consisting of selected from the group consisting of (4S,5S)-3,4-dihydroxy-4-[(1S)-1-hydroxy-4-methylpentyl]-2-(3-methylbutanoyl)-5-(3-methylbutyl)cyclopent-2-en-1-one, (4S,5S)-3,4-dihydroxy-4-[(1R)-1-hydroxy-4-methylpentyl]-2-(3-methylbutanoyl)-5-(3-methylbutyl)cyclopent-2-en-1-one, (4R,5S)-3,4-dihydroxy-4-[(1R)-1-hydroxy-4-methylpentyl]-2-(3-methylbutanoyl)-5-(3-methylbutyl)cyclopent-2-en-1-one, (4R,5S)-3,4-dihydroxy-4-[(1S)-1-hydroxy-4-methylpentyl]-2-(3-methylbutanoyl)-5-(3-methylbutyl)cyclopent-2-en-1-one, (4R,5R)-3,4-dihydroxy-4-[(1R)-1-hydroxy-4-methylpentyl]-2-(3-methylbutanoyl)-5-(3-methylbutyl)cyclopent-2-en-1-one, (4R,5R)-3,4-dihydroxy-4-[(1S)-1-hydroxy-4-methylpentyl]-2-(3-methylbutanoyl)-5-(3-methylbutyl)cyclopent-2-en-1-one, (4S,5R)-3,4-dihydroxy-4-[(1S)-1-hydroxy-4-methylpentyl]-2-(3-methylbutanoyl)-5-(3-methylbutyl)cyclopent-2-en-1-one, (4S,5R)-3,4-dihydroxy-4-[(1R)-1-hydroxy-4-methylpentyl]-2-(3-methylbutanoyl)-5-(3-methylbutyl)cyclopent-2-en-1-one, (4S,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4S,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4R,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5R)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4S,5R)-3,4-dihydroxy-4-[(1S)-hydroxy-4-methylpent-3-en-1-yl]-2-(2-methylpropanoyl)-5-(3-methylbut-2-en-1-yl)cyclopent-2-en-1-one, (4S,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4S,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4S,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4R,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4S,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, and (4S,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one.

20. The composition according to claim 13, wherein the solvent for the bi-phasic system is selected from the group comprising water, water containing a buffering agent, water containing a soluble polymer, a pentane, hexane, heptane, octane, methyl acetate, ethyl acetate, propyl acetate, butyl acetate, tert-butyl acetate, methanol, ethanol, propanol, iso propanol, butanol, tert butanol, dimethyl formamide, dimethyl sulfoxide, dichloromethane, chloroform; and acetone, or any combination thereof.

21. The composition of claim 13, wherein the bi-phasic solvent system has a partition coefficient between 0.6 and 3.0.

22. The composition of claim 13, wherein the bi-phasic solvent system has a partition coefficient between 0.7 and 1.5.

23. The composition of claim 13, wherein the counter current chromatography is performed at a temperature of about 20° C. to about 30° C.

24. The composition of claim 13, wherein the counter current chromatography is performed at ambient temperature.

25. The composition of any of claims 13 to 24, wherein pharmaceutically acceptable excipient is selected from the group consisting of an isotonic and absorption delaying agent, binder, adhesive, lubricant, disintegrant, coloring agent, flavoring agent, sweetening agent, absorbants, detergent, and emulsifying agent, or any combination thereof.

26. The composition of any of claims 13 to 24, wherein said composition further comprises one or more antioxidants, vitamins, minerals, proteins, fats, and carbohydrates.

27. The composition of any of claims 13 to 26, wherein said composition is in a dosage form suitable for administration via a route selected from the group consisting of oral, inhalation, rectal, ophthalmic, nasal, topical, vaginal, and parenteral.

28. The composition according to claim 13, wherein the congener is at least eighty percent pure.

29. The composition according to claim 13, wherein the congener is at least ninety-five percent pure.

30. A method for isolating a congener in substantially pure form from a mixture of congeners comprising the steps of:
a. preparing a suitable mixture of individual congeners derived from an extract of hops;
b. dissolving said mixture with a suitable solvent that can be introduced into a counter current separation instrument for the purpose of purification;
c. collecting a homogenous or partially homogenous solution of individual congeners;
d. extracting the counter current separation purified congeners into a suitable solvent obtained from step (c); or
e. removing the solvent of the solution obtained in step (c) of a homogenous or partially homogenous solution of an individual congener to render the pure or partially pure congener; and
f. removing the solvent of the solution obtained in step (d) of a homogenous or partially homogenous solution of an individual congener to render the pure or partially pure congener;
wherein said congener is selected from the group consisting of substituted cyclohexa-2,4-dienones, substituted cyclohexane-1,3,5-triones, substituted cyclopent-2-en-1-ones, and substituted 1,3-cyclopentadiones.

31. The method of claim 30, wherein the substituted cyclohexa-2,4-dienone is selected from the group consisting of (6R)-3,5,6-trihydroxy-2-(3-methylbutanoyl)-4,6-bis(3-methylbut-2-en-1-yl)cyclohexa-2,4-dien-1-one, (6R)-3,5,6-trihydroxy-4,6-bis(3-methylbut-2-en-1-yl)-2-(2-methylpropanoyl)cyclohexa-2,4-dien-1-one, and (6R)-3,5,6-trihydroxy-2-(2-methylbutanoyl)-4,6-bis(3-methylbut-2-en-1-yl)cyclohexa-2,4-dien-1-one.

32. The method of claim 30, wherein the substituted cyclohexane-1,3,5-trione is selected from the group consisting of substituted cyclohexane-1,3,5-triones wherein said composition is enriched for one or more compounds selected from the group consisting of 3,5-dihydroxy-2-(3-methylbutanoyl)-4,6,6-tris(3-methylbut-2-en-1-yl)cyclohexa-2,4-dien-1-one, 3,5-dihydroxy-4,6,6-tris(3-methylbut-2-en-1-yl)-2-(2-methylpropanoyl)cyclohexa-2,4-dien-1-one, and 3,5-dihydroxy-2-(2-methylbutanoyl)-4,6,6-tris(3-methylbut-2-en-1-yl)cyclohexa-2,4-dien-1-one.

33. The method of claim 30, wherein the substituted cyclopent-2-en-1-one is selected from the group consisting of (4R,5S)-4-hydroxy-3-methyl-2-(3-methylbutanoyl)-5-(3-methylbut-2-en-1-yl)-4-(4-methylpent-3-enoyl)cyclopent-2-en-1-one, (4S,5S)-4-hydroxy-3-methyl-2-(3-methylbutanoyl)-5-(3-methylbut-2-en-1-yl)-4-(4-methylpent-3-enoyl)cyclopent-2-en-1-one, (4S,5R)-4-hydroxy-3-methyl-2-(3-methylbutanoyl)-5-(3-methylbut-2-en-1-yl)-4-(4-methylpent-3-enoyl)cyclopent-2-en-1-one, (4R,5R)-4-hydroxy-3-methyl-2-(3-methylbutanoyl)-5-(3-methylbut-2-en-1-yl)-4-(4-methylpent-3-enoyl)cyclopent-2-en-1-one, (4R,5S)-4-hydroxy-3-methyl-5-(3-methylbut-2-en-1-yl)-4-(4-methylpent-3-enoyl)-2-(2-methylpropanoyl)cyclopent-2-en-1-one, (4S,5S)-4-hydroxy-3-methyl-5-(3-methylbut-2-en-1-yl)-4-(4-methylpent-3-enoyl)-2-(2-methylpropanoyl)cyclopent-2-en-1-one, (4S,5R)-4-hydroxy-3-methyl-5-(3-methylbut-2-en-1-yl)-4-(4-methylpent-3-enoyl)-2-(2-methylpropanoyl)cyclopent-2-en-1-one, (4R,5R)-4-hydroxy-3-methyl-5-(3-methylbut-2-en-1-yl)-4-(4-methylpent-3-enoyl)-2-(2-methylpropanoyl)cyclopent-2-en-1-one, (4R,5S)-4-hydroxy-3-methyl-2-(2-methylbutanoyl)-5-(3-methylbut-2-en-1-yl)-4-(4-methylpent-3-enoyl)cyclopent-2-en-1-one, (4S,5S)-4-hydroxy-3-methyl-2-(2-methylbutanoyl)-5-(3-methylbut-2-en-1-yl)-4-(4-methylpent-3-enoyl)cyclopent-2-en-1-one, (4S,5R)-4-hydroxy-3-methyl-2-(2-methylbutanoyl)-5-(3-methylbut-2-en-1-yl)-4-(4-methylpent-3-enoyl)cyclopent-2-en-1-one, and (4R,5R)-4-hydroxy-3-methyl-2-(2-methylbutanoyl)-5-(3-methylbut-2-en-1-yl)-4-(4-methylpent-3-enoyl)cyclopent-2-en-1-one.

34. The method of claim 30, wherein the substituted 1,3-cyclopentadione is selected from the group consisting of (4S,5S)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5S)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5S)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5S)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4R,5R)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5R)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5R)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5R)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4S,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4S,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4R,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5R)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4S,5R)-3,4-dihydroxy-4-[(1S)-hydroxy-4-methylpent-3-en-1-yl]-2-(2-methylpropanoyl)-5-(3-methylbut-2-en-1-yl)cyclopent-2-en-1-one; (4S,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4S,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4S,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4R,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4S,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; and to 99.9 percent by weight of (4S,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one.

35. The method of claim 30, wherein the substituted 1,3-cyclopentadione is selected from the group consisting of selected from the group consisting of (4R,5S)-3,4-dihydroxy-2-(3-methylbutanoyl)-5-(3-methylbutyl)-4-(4-methylpentanoyl)cyclopent-2-en-1-one, (4S,5S)-3,4-dihydroxy-2-(3-methylbutanoyl)-5-(3-methylbutyl)-4-(4-methylpentanoyl)cyclopent-2-en-1-one, (4S,5R)-3,4-dihydroxy-2-(3-methylbutanoyl)-5-(3-methylbutyl)-4-(4-methylpentanoyl)cyclopent-2-en-1-one, (4R,5R)-3,4-dihydroxy-2-(3-methylbutanoyl)-5-(3-methylbutyl)-4-(4-methylpentanoyl)cyclopent-2-en-1-one, (4R,5S)-3,4-dihydroxy-5-(3-methylbutyl)-4-(4-methylpentanoyl)-2-(3-methylpropanoyl)cyclopent-2-en-1-one, (4S,5S)-3,4-dihydroxy-5-(3-methylbutyl)-4-(4-methylpentanoyl)-2-(3-methylpropanoyl)cyclopent-2-en-1-one, (4S,5R)-3,4-dihydroxy-5-(3-methylbutyl)-4-(4-methylpentanoyl)-2-(3-methylpropanoyl)cyclopent-2-en-1-one, (4R,5R)-3,4-dihydroxy-5-(3-methylbutyl)-4-(4-methylpentanoyl)-2-(3-methylpropanoyl)cyclopent-2-en-1-one, (4R,5S)-3,4-dihydroxy-2-(2-methylbutanoyl)-5-(3-methylbutyl)-4-(4-methylpentanoyl)cyclopent-2-en-1-one, (4S,5S)-3,4-dihydroxy-2-(2-methylbutanoyl)-5-(3-methylbutyl)-4-(4-methylpentanoyl)cyclopent-2-en-1-one, (4S,5R)-3,4-dihydroxy-2-(2-methylbutanoyl)-5-(3-methylbutyl)-4-(4-methylpentanoyl)cyclopent-2-en-1-one, and (4R,5R)-3,4-dihydroxy-2-(2-methylbutanoyl)-5-(3-methylbutyl)-4-(4-methylpentanoyl)cyclopent-2-en-1-one.

36. The method of claim 30, wherein the substituted 1,3-cyclopentadione is selected from the group consisting of selected from the group consisting of (4S,5S)-3,4-dihydroxy-4-[(1S)-1-hydroxy-4-methylpentyl]-2-(3-methylbutanoyl)-5-(3-methylbutyl)cyclopent-2-en-1-one, (4S,5S)-3,4-dihydroxy-4-[(1R)-1-hydroxy-4-methylpentyl]-2-(3-methylbutanoyl)-5-(3-methylbutyl)cyclopent-2-en-1-one, (4R,5S)-3,4-dihydroxy-4-[(1R)-1-hydroxy-4-methylpentyl]-2-(3-methylbutanoyl)-5-(3-methylbutyl)cyclopent-2-en-1-one, (4R,5S)-3,4-dihydroxy-4-[(1S)-1-hydroxy-4-methylpentyl]-2-(3-methylbutanoyl)-5-(3-methylbutyl)cyclopent-2-en-1-one, (4R,5R)-3,4-dihydroxy-4-[(1R)-1-hydroxy-4-methylpentyl]-2-(3-methylbutanoyl)-5-(3-methylbutyl)cyclopent-2-en-1-one, (4R,5R)-3,4-dihydroxy-4-[(1S)-1-hydroxy-4-methylpentyl]-2-(3-methylbutanoyl)-5-(3-methylbutyl)cyclopent-2-en-1-one, (4S,5R)-3,4-dihydroxy-4-[(1S)-1-hydroxy-4-methylpentyl]-2-(3-methylbutanoyl)-5-(3-methylbutyl)cyclopent-2-en-1-one, (4S,5R)-3,4-dihydroxy-4-[(1R)-1-hydroxy-4-methylpentyl]-2-(3-methylbutanoyl)-5-(3-methylbutyl)cyclopent-2-en-1-one, (4S,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4S,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4R,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5R)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4S,5R)-3,4-dihydroxy-4-[(1S)-hydroxy-4-methylpent-3-en-1-yl]-2-(2-methylpropanoyl)-5-(3-methylbut-2-en-1-yl)cyclopent-2-en-1-one, (4S,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4S,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4S,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4R,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4S,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, and (4S,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one.

37. The method according to claim 30, wherein the solvent for the bi-phasic system is selected from the group comprising water, water containing a buffering agent, water containing a soluble polymer, a pentane, hexane, heptane, octane, methyl acetate, ethyl acetate, propyl acetate, butyl acetate, tert-butyl acetate, methanol, ethanol, propanol, iso propanol, butanol, tert butanol, dimethyl formamide, dimethyl sulfoxide, dichloromethane, chloroform; and acetone, or any combination thereof.

38. The method of claim 30, wherein the bi-phasic solvent system has a partition coefficient between 0.6 and 3.0.

39. The method of claim 30, wherein the bi-phasic solvent system has a partition coefficient between 0.7 and 1.5.

40. The method of claim 30, wherein the counter current chromatography is performed at a temperature of about 20° C. to about 30° C.

41. The method of claim 30, wherein the counter current chromatography is performed at ambient temperature.

42. A composition comprising a substantially pure congener or a pharmaceutically acceptable salt thereof, wherein said congener is obtained from a mixture of congeners comprising the steps of:
a. preparing a suitable mixture of individual congeners derived from an extract of hops;
b. dissolving said mixture with a suitable solvent that can be introduced into a counter current separation instrument for the purpose of purification;
c. collecting a homogenous or partially homogenous solution of individual congeners;
d. extracting the counter current separation purified congeners into a suitable solvent obtained from step (c); or
e. removing the solvent of the solution obtained in step (c) of a homogenous or partially homogenous solution of an individual congener to render the pure or partially pure congener; and
f. removing the solvent of the solution obtained in step (d) of a homogenous or partially homogenous solution of an individual congener to render the pure or partially pure congener;
wherein said congener is selected from the group consisting of substituted cyclohexa-2,4-dienones, substituted cyclohexane-1,3,5-triones, substituted cyclopent-2-en-1-ones, and substituted 1,3-cyclopentadiones; and
wherein said composition further comprises a pharmaceutically acceptable excipient.

43. The composition of claim 42, wherein the substituted cyclohexa-2,4-dienone is selected from the group consisting of (6R)-3,5,6-trihydroxy-2-(3-methylbutanoyl)-4,6-bis(3-methylbut-2-en-1-yl)cyclohexa-2,4-dien-1-one, (6R)-3,5,6-trihydroxy-4,6-bis(3-methylbut-2-en-1-yl)-2-(2-methylpropanoyl)cyclohexa-2,4-dien-1-one, and (6R)-3,5,6-trihydroxy-2-(2-methylbutanoyl)-4,6-bis(3-methylbut-2-en-1-yl)cyclohexa-2,4-dien-1-one.

44. The composition of claim 42, wherein the substituted cyclohexane-1,3,5-trione is selected from the group consisting of substituted cyclohexane-1,3,5-triones wherein said composition is enriched for one or more compounds selected from the group consisting of 3,5-dihydroxy-2-(3-methylbutanoyl)-4,6,6-tris(3-methylbut-2-en-1-yl)cyclohexa-2,4-dien-1-one, 3,5-dihydroxy-4,6,6-tris(3-methylbut-2-en-1-yl)-2-(2-methylpropanoyl)cyclohexa-2,4-dien-1-one, and 3,5-dihydroxy-2-(2-methylbutanoyl)-4,6,6-tris(3-methylbut-2-en-1-yl)cyclohexa-2,4-dien-1-one.

45. The composition of claim 42, wherein the substituted cyclopent-2-en-1-one is selected from the group consisting of (4R,5S)-4-hydroxy-3-methyl-2-(3-methylbutanoyl)-5-(3-methylbut-2-en-1-yl)-4-(4-methylpent-3-enoyl)cyclopent-2-en-1-one, (4S,5S)-4-hydroxy-3-methyl-2-(3-methylbutanoyl)-5-(3-methylbut-2-en-1-yl)-4-(4-methylpent-3-enoyl)cyclopent-2-en-1-one, (4S,5R)-4-hydroxy-3-methyl-2-(3-methylbutanoyl)-5-(3-methylbut-2-en-1-yl)-4-(4-methylpent-3-enoyl)cyclopent-2-en-1-one, (4R,5R)-4-hydroxy-3-methyl-2-(3-methylbutanoyl)-5-(3-methylbut-2-en-1-yl)-4-(4-methylpent-3-enoyl)cyclopent-2-en-1-one, (4R,5S)-4-hydroxy-3-methyl-5-(3-methylbut-2-en-1-yl)-4-(4-methylpent-3-enoyl)-2-(2-methylpropanoyl)cyclopent-2-en-1-one, (4S,5S)-4-hydroxy-3-methyl-5-(3-methylbut-2-en-1-yl)-4-(4-methylpent-3-enoyl)-2-(2-methylpropanoyl)cyclopent-2-en-1-one, (4S,5R)-4-hydroxy-3-methyl-5-(3-methylbut-2-en-1-yl)-4-(4-methylpent-3-enoyl)-2-(2-methylpropanoyl)cyclopent-2-en-1-one, (4R,5R)-4-hydroxy-3-methyl-5-(3-methylbut-2-en-1-yl)-4-(4-methylpent-3-enoyl)-2-(2-methylpropanoyl)cyclopent-2-en-1-one, (4R,5S)-4-hydroxy-3-methyl-2-(2-methylbutanoyl)-5-(3-methylbut-2-en-1-yl)-4-(4-methylpent-3-enoyl)cyclopent-2-en-1-one, (4S,5S)-4-hydroxy-3-methyl-2-(2-methylbutanoyl)-5-(3-methylbut-2-en-1-yl)-4-(4-methylpent-3-enoyl)cyclopent-2-en-1-one, (4S,5R)-4-hydroxy-3-methyl-2-(2-methylbutanoyl)-5-(3-methylbut-2-en-1-yl)-4-(4-methylpent-3-enoyl)cyclopent-2-en-1-one, and (4R,5R)-4-hydroxy-3-methyl-2-(2-methylbutanoyl)-5-(3-methylbut-2-en-1-yl)-4-(4-methylpent-3-enoyl)cyclopent-2-en-1-one.

46. The composition of claim 42, wherein the substituted 1,3-cyclopentadione is selected from the group consisting of (4S,5S)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5S)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5S)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5S)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4R,5R)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4R,5R)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5R)-3,4-dihydroxy-4-[(1S)-hydroxy-4-methylpent-3-en-1-yl]-2-(3-methylbutanoyl)-5-(3-methylbut-2-en-1-yl)cyclopent-2-en-1-one; (4S,5R)-3,4-dihydroxy-4-[(1R)-hydroxy-4-methylpent-3-en-1-yl]-2-(3-methylbutanoyl)-5-(3-methylbut-2-en-1-yl)cyclopent-2-en-1-one; (4S,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4S,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4R,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4R,5R)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one; (4S,5R)-3,4-dihydroxy-4-[(1S)-hydroxy-4-methylpent-3-en-1-yl]-2-(2-methylpropanoyl)-5-(3-methylbut-2-en-1-yl)cyclopent-2-en-1-one; (4S,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one; (4S,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4S,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4R,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one; (4R,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; (4S,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one; and to 99.9 percent by weight of (4S,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one.

47. The composition of claim 42, wherein the substituted 1,3-cyclopentadione is selected from the group consisting of selected from the group consisting of (4R,5S)-3,4-dihydroxy-2-(3-methylbutanoyl)-5-(3-methylbutyl)-4-(4-methylpentanoyl)cyclopent-2-en-1-one, (4S,5S)-3,4-dihydroxy-2-(3-methylbutanoyl)-5-(3-methylbutyl)-4-(4-methylpentanoyl)cyclopent-2-en-1-one, (4S,5R)-3,4-dihydroxy-2-(3-methylbutanoyl)-5-(3-methylbutyl)-4-(4-methylpentanoyl)cyclopent-2-en-1-one, (4R,5R)-3,4-dihydroxy-2-(3-methylbutanoyl)-5-(3-methylbutyl)-4-(4-methylpentanoyl)cyclopent-2-en-1-one, (4R,5S)-3,4-dihydroxy-5-(3-methylbutyl)-4-(4-methylpentanoyl)-2-(3-methylpropanoyl)cyclopent-2-en-1-one, (4S,5S)-3,4-dihydroxy-5-(3-methylbutyl)-4-(4-methylpentanoyl)-2-(3-methylpropanoyl)cyclopent-2-en-1-one, (4S,5R)-3,4-dihydroxy-5-(3-methylbutyl)-4-(4-methylpentanoyl)-2-(3-methylpropanoyl)cyclopent-2-en-1-one, (4R,5R)-3,4-dihydroxy-5-(3-methylbutyl)-4-(4-methylpentanoyl)-2-(3-methylpropanoyl)cyclopent-2-en-1-one, (4R,5S)-3,4-dihydroxy-2-(2-methylbutanoyl)-5-(3-methylbutyl)-4-(4-methylpentanoyl)cyclopent-2-en-1-one, (4S,5S)-3,4-dihydroxy-2-(2-methylbutanoyl)-5-(3-methylbutyl)-4-(4-methylpentanoyl)cyclopent-2-en-1-one, (4S,5R)-3,4-dihydroxy-2-(2-methylbutanoyl)-5-(3-methylbutyl)-4-(4-methylpentanoyl)cyclopent-2-en-1-one, and (4R,5R)-3,4-dihydroxy-2-(2-methylbutanoyl)-5-(3-methylbutyl)-4-(4-methylpentanoyl)cyclopent-2-en-1-one.

48. The composition of claim 42, wherein the substituted 1,3-cyclopentadione is selected from the group consisting of selected from the group consisting of (4S,5S)-3,4-dihydroxy-4-[(1S)-1-hydroxy-4-methylpentyl]-2-(3-methylbutanoyl)-5-(3-methylbutyl)cyclopent-2-en-1-one, (4S,5S)-3,4-dihydroxy-4-[(1R)-1-hydroxy-4-methylpentyl]-2-(3-methylbutanoyl)-5-(3-methylbutyl)cyclopent-2-en-1-one, (4R,5S)-3,4-dihydroxy-4-[(1R)-1-hydroxy-4-methylpentyl]-2-(3-methylbutanoyl)-5-(3-methylbutyl)cyclopent-2-en-1-one, (4R,5S)-3,4-dihydroxy-4-[(1S)-1-hydroxy-4-methylpentyl]-2-(3-methylbutanoyl)-5-(3-methylbutyl)cyclopent-2-en-1-one, (4R,5R)-3,4-dihydroxy-4-[(1R)-1-hydroxy-4-methylpentyl]-2-(3-methylbutanoyl)-5-(3-methylbutyl)cyclopent-2-en-1-one, (4R,5R)-3,4-dihydroxy-4-[(1S)-1-hydroxy-4-methylpentyl]-2-(3-methylbutanoyl)-5-(3-methylbutyl)cyclopent-2-en-1-one, (4S,5R)-3,4-dihydroxy-4-[(1S)-1-hydroxy-4-methylpentyl]-2-(3-methylbutanoyl)-5-(3-methylbutyl)cyclopent-2-en-1-one, (4S,5R)-3,4-dihydroxy-4-[(1R)-1-hydroxy-4-methylpentyl]-2-(3-methylbutanoyl)-5-(3-methylbutyl)cyclopent-2-en-1-one, (4S,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4S,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5S)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5S)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4R,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4R,5R)-3,4-dihydroxy-4-[(1S)-hydroxy-4-methylpent-3-en-1-yl]-5-(3-methylbut-2-en-1-yl)-2-(2-methylpropanoyl)cyclopent-2-en-1-one, (4S,5R)-3,4-dihydroxy-4-[(1S)-hydroxy-4-methylpent-3-en-1-yl]-2-(2-methylpropanoyl)-5-(3-methylbut-2-en-1-yl)cyclopent-2-en-1-one, (4S,5R)-3,4-dihydroxy-4-[(1R)-hydroxy-4-methylpent-3-en-1-yl]-5-(3-methylbut-2-en-1-yl)-2-(2-methylpropanoyl)cyclopent-2-en-1-one, (4S,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4S,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5S)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5S)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4R,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one, (4R,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, (4S,5R)-3,4-dihydroxy-4-[(1S)-hydroxy-4-methylpent-3-en-1-yl]-2-(2-methylbutanoyl)-5-(3-methylbut-2-en-1-yl)cyclopent-2-en-1-one, and (4S,5R)-3,4-dihydroxy-4-[(1R)-hydroxy-4-methylpent-3-en-1-yl]-2-(2-methylbutanoyl)-5-(3-methylbut-2-en-1-yl)cyclopent-2-en-1-one.

49. The composition according to claim 42, wherein the solvent for the bi-phasic system is selected from the group comprising water, water containing a buffering agent, water containing a soluble polymer, a pentane, hexane, heptane, octane, methyl acetate, ethyl acetate, propyl acetate, butyl acetate, tert-butyl acetate, methanol, ethanol, propanol, iso propanol, butanol, tert butanol, dimethyl formamide, dimethyl sulfoxide, dichloromethane, chloroform; and acetone, or any combination thereof.

50. The composition of claim 42, wherein the bi-phasic solvent system has a partition coefficient between 0.6 and 3.0.

51. The composition of claim 42, wherein the bi-phasic solvent system has a partition coefficient between 0.7 and 1.5.

52. The composition of claim 42, wherein the counter current chromatography is performed at a temperature of about 20° C. to about 30° C.

53. The composition of claim 42, wherein the counter current chromatography is performed at ambient temperature.

54. The composition of any of claims 42 to 53, wherein said pharmaceutically acceptable excipient is selected from the group consisting of an isotonic and absorption delaying agent, binder, adhesive, lubricant, disintegrant, coloring agent, flavoring agent, sweetening agent, absorbants, detergent, and emulsifying agent, or any combination thereof.

55. The composition of any of claims 42 to 51, wherein said composition further comprises one or more antioxidants, vitamins, minerals, proteins, fats, and carbohydrates.

56. The composition of any of claims 42 to 55, wherein said composition is in a dosage form suitable for administration via a route selected from the group consisting of oral, inhalation, rectal, ophthalmic, nasal, topical, vaginal, and parenteral.

57. The composition according to claim 42, wherein the congener is at least eighty percent pure.

58. The composition according to claim 42, wherein the congener is at least ninety-five percent pure.
